(19) United States
(12) Patent Application Publication
(10) Pub. No.: US 2009/0105167 A1
(43) Pub. Date: Apr. 23, 2009

(54) Predicting responsiveness to cancer therapeutics

(75) Inventors: Anil Potti, Chapel Hill, NC (US); Joseph R. Nevins, Chapel Hill, NC (US); Johnathan M. Lancaster, Tampa, FL (US)

Correspondence Address: MICHAEL BEST & FRIEDRICH LLP, 100 E WISCONSIN AVENUE, Suite 3300, MILWAUKEE, WI, 53202, US

(73) Assignee: Duke University, Durham, NC (US)

(21) Appl. No.: 11/975,722

(22) Filed: Oct. 19, 2007

(51) Int. Cl. A61K 31/7048; C12Q 1/68; C40B 40/08; A61P 43/00; G06G 7/48; A61K 31/44

(52) U.S. Cl. 514/34; 435/6; 514/283; 506/17; 703/11

(57) ABSTRACT

The invention provides for compositions and methods for predicting an individual's responsitivity to cancer treatments and methods of treating cancer. In certain embodiments, the invention provides compositions and methods for predicting an individual's responsitivity to chemotherapeutics, including salvage agents, to treat cancers such as ovarian cancer. The invention also provides reagents, such as DNA microarrays, software and computer systems useful for personalizing cancer treatments, and provides methods of conducting a diagnostic business for personalizing cancer treatments.

### STATEMENT REGARDING FEDERALLY SPONSORED RESEARCH OR DEVELOPMENT

This invention was made with government support under NCI-U54 CA1112952-02 and R01-CA106520 awarded by the National Cancer Institute. The government has certain rights in the invention.

### FIELD OF THE INVENTION

Cancer therapeutics are often effective only in a subset of patients. In addition, chemotherapeutic drugs often have toxic side effects. To address this problem, it will be useful to predict which cancer therapeutics will be effective for a given patient. This invention relates to a gene predictor set wherein altered expression of certain genes is correlated with high or low responsiveness to chemotherapeutic drugs. A tumor sample is collected from a patient and its gene expression profile is determined. This profile is then compared to a gene predictor set. This comparison allows one to select the therapy that is most likely to be effective for the individual patient.

### BACKGROUND OF THE INVENTION

Numerous advances in the development, selection, and application of chemotherapy agents, sometimes with remarkable successes as seen in the case of treatment for lymphomas or platinum-based therapy for testicular cancers (Herbst, R. S. et al. Clinical Cancer Advances 2005; major research advances in cancer treatment, prevention, and screening—a report from the American Society of Clinical Oncology. J. Clin. Oncol. 24, 190-205 (2006)). In addition, in several instances, combination chemotherapy in the adjuvant setting has been found to be curative. However, most patients with clinically or pathologically advanced solid tumors will relapse and die of their disease. Moreover, administration of ineffective chemotherapy increases the probability of side-effects, particularly from cytotoxic agents, and consequently a decrease in quality of life (Herbst, R. S. et al. Clinical Cancer Advances 2005; major research advances in cancer treatment, prevention, and screening—a report from the American Society of Clinical Oncology. J. Clin. Oncol. 24, 190-205 (2006), Breathnach, O. S. et al. Twenty-two years of phase III trials for patients with advanced non-small-cell lung cancer: sobering results. J. Clin. Oncol. 19, 1734-1742 (2001).).

Recent work has demonstrated the value in the use of biomarkers to select patients for various targeted therapeutics including tamoxifen, trastuzumab, and imatinib mesylate. In contrast, equivalent tools to select those patients most likely to respond to the commonly used chemotherapeutic drugs are lacking. A thorough understanding of drug resistance mechanisms should provide insight into how best to overcome resistance and, more importantly, the development of a strategy to match patients with drugs to which they are most likely to be sensitive and/or identify appropriate drug combinations for individual patient/patient groups is critical.

Throughout this specification, reference numbering is sometimes used to refer to the full citation for the references, which can be found in the “Reference Bibliography” after the Examples section. The disclosure of all patents, patent applications, and publications cited herein are hereby incorporated by reference in their entirety for all purposes.

### BRIEF SUMMARY OF THE INVENTION

In one aspect, the invention provides a method of identifying an effective cancer therapy agent for an individual with a platinum-resistant tumor, comprising: (a) obtaining a cellular sample from the individual; (b) analyzing said sample to obtain a first gene expression profile; (c) comparing said first gene expression profile to a platinum chemotherapy responsivity predictor set of gene expression profiles to identify whether said individual will be responsive to a platinum-based therapy; (d) if said individual is an incomplete responder to platinum based therapy, then comparing the first gene expression profile to a set of gene expression profiles comprising at least 5 genes from Table 1 that is capable of predicting responsiveness to other cancer therapy agents; thereby identifying whether said individual would benefit from the administration of one or more cancer therapy agents wherein said cancer therapy agents are not platinum-based.

In another aspect, the invention provides a method of treating an individual with ovarian cancer comprising: (a) obtaining a cellular sample from the individual; (b) analyzing said sample to obtain a first gene expression profile; (c) comparing said first gene expression profile to a platinum chemotherapy responsivity predictor set of gene expression profiles to identify whether said individual will be responsive to a platinum-based therapy; (d) if said individual is a complete responder or incomplete responder, then administering an effective amount of platinum-based therapy to the individual; (e) if said individual is predicted to be an incomplete responder to platinum based therapy, then comparing the first gene expression profile to a set of gene expression profiles comprising at least 5 genes from Table 1 that is predictive of responsivity to additional cancer therapeutics to identify to which additional cancer therapeutic the individual would be responsive; and (f) administering to said individual an effective amount of one or more of the additional cancer therapeutic that was identified in step (e); thereby treating the individual with ovarian cancer.

In certain embodiments, the cellular sample is taken from a tumor sample or ascites. In certain embodiments the set of gene expression profiles that is capable of predicting responsiveness to salvage therapy agents comprises at least 10 or 15 genes from Table 1. The cancer therapy agent may be a salvage therapy agent. In addition, the salvage therapy agent may be selected from the group consisting of topotecan, adriamycin, doxorubicin, cytoxan, cyclophosphamide, gemcitabine, etoposide, ifosfamide, paclitaxel, docetaxel, and taxol. Furthermore, the cancer therapy agent may target a signal transduction pathway that is deregulated. The cancer therapy agent may be selected from the group consisting of inhibitors of the Src pathway, inhibitors of the E2F3 pathway, inhibitors of the Myc pathway, and inhibitors of the beta-catenin pathway. In one embodiment, the platinum-based therapy is administered first, followed by the administration of one or more salvage therapy agent. The platinum-based therapy may also be administered concurrently with one or more salvage therapy agent. One or more salvage therapy agent may be administered by itself. Alternatively, the salvage therapy agent may be administered first, followed by the administration of one or more platinum-based therapy.

In yet another aspect, the invention provides for a gene chip for predicting an individual's responsivity to a salvage therapy agent comprising the gene expression profile of at least 5 genes selected from Table 1.

In yet another aspect, the invention provides for a gene chip for predicting an individual's responsivity to a salvage therapy agent comprising the gene expression profile of at least 10 genes selected from Table 1.

In yet another aspect, the invention provides for a gene chip for predicting an individual's responsivity to a salvage therapy agent comprising the gene expression profile of at least 20 genes selected from Table 1.

In yet another aspect, the invention provides for a kit comprising a gene chip for predicting an individual's responsivity to a salvage therapy agent and a set of instructions for determining an individual's responsivity to salvage chemotherapy agents.

In yet another aspect, the invention provides for a computer readable medium comprising gene expression profiles comprising at least 5 genes from any of Table 1.

In yet another aspect, the invention provides for a computer readable medium comprising gene expression profiles comprising at least 15 genes from Table 5.

In yet another aspect, the invention provides for a computer readable medium comprising gene expression profiles comprising at least 25 genes from Table 5.

In yet another aspect, the invention provides a method for estimating or predicting the efficacy of a therapeutic agent in treating an individual afflicted with cancer. In one aspect, the method comprises: (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more statistical tree models applied to the values of the metagenes, wherein each model includes one or more nodes, each node representing a metagene, each node including a statistical predictive probability of tumor sensitivity to the therapeutic agent, wherein at least one of the metagenes comprises at least 3 genes in metagenes 1, 2, 3, 4, 5, 6, or 7, thereby estimating the efficacy of a therapeutic agent in an individual afflicted with cancer. In certain embodiments, step (a) comprises extracting a nucleic acid sample from the sample from the subject. In certain embodiments, the method further comprising: (d) detecting the presence of pathway deregulation by comparing the expression levels of the genes to one or more reference profiles indicative of pathway deregulation, and (e) selecting an agent that is predicted to be effective and regulates a pathway deregulated in the tumor. In certain embodiments said pathway is selected from RAS, SRC, MYC, E2F, and β-catenin pathways.

In yet another aspect, the invention provides a method for estimating the efficacy of a therapeutic agent in treating an individual afflicted with cancer. In one aspect, the method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more binary regression models applied to the values of the metagenes, wherein each model includes a statistical predictive probability of tumor sensitivity to the therapeutic agent, wherein at least one of the metagenes comprises at least 3 genes in metagene 1, 2, 3, 4, 5, 6, or 7, thereby estimating the efficacy of a therapeutic agent in an individual afflicted with cancer.

In yet another aspect, the invention provides a method of treating an individual afflicted with cancer, said method comprising: (a) estimating the efficacy of a plurality of therapeutic agents in treating an individual afflicted with cancer according to the methods if the invention; (b) selecting a therapeutic agent having the high estimated efficacy; and (c) administering to the subject an effective amount of the selected therapeutic agent, thereby treating the subject afflicted with cancer. The method of estimating the efficacy may comprise (i) determining the expression level of multiple genes in a tumor biopsy sample from the subject and (ii) averaging the predictions of one or more statistical tree models applied to the values of one or more of metagenes 1, 2, 3, 4, 5, 6, and 7, wherein each model includes one or more nodes, each node representing a metagene, each node including a statistical predictive probability of tumor sensitivity to the therapeutic agent.

In yet another aspect, the invention provides a therapeutic agent having the high estimated efficacy is one having an estimated efficacy in treating the subject of at least 50%. In certain embodiments, the invention provides a therapeutic agent having the high estimated efficacy is one having an estimated efficacy in treating the subject of at least 80%.

In certain embodiments, the tumor is selected from a breast tumor, an ovarian tumor, and a lung tumor. In certain embodiments, the therapeutic agent is selected from docetaxel, paclitaxel, topotecan, adriamycin, etoposide, fluorouracil (5-FU), and cyclophosphamide, or any combination thereof.

In certain embodiments, the therapeutic agent is docetaxel and wherein the cluster of genes comprises at least 10 genes from metagene 1. In certain embodiments, the therapeutic agent is paclitaxel, and wherein the cluster of genes comprises at least 10 genes from metagene 2. In certain embodiments, wherein the therapeutic agent is topotecan, and wherein the cluster of genes comprises at least 10 genes from metagene 3. In certain embodiments, wherein the therapeutic agent is adriamycin, and wherein the cluster of genes comprises at least 10 genes from metagene 4. In certain embodiments, wherein the therapeutic agent is etoposide, and wherein the cluster of genes comprises at least 10 genes from metagene 5. In certain embodiments, wherein the therapeutic agent is fluorouracil (5-FU), and wherein the cluster of genes comprises at least 10 genes from metagene 6. In certain embodiments, wherein the therapeutic agent is cyclophosphamide and wherein the cluster of genes comprises at least 10 genes from metagene 7.

In certain embodiments, at least one of the metagenes is metagene 1, 2, 3, 4, 5, 6, or 7. In certain embodiments, the cluster of genes corresponding to at least one of the metagenes comprises 3 or more genes in common to metagene 1, 2, 3, 4, 5, 6, or 7. In certain embodiments, the cluster of genes corresponding to at least one metagene comprises 5 or more genes in common to metagene 1, 2, 3, 4, 5, 6, or 7. In certain embodiments, the cluster of genes corresponding to at least one metagene comprises at least 10 genes, wherein half or more of the genes are common to metagene 1, 2, 3, 4, 5, 6, or 7.

In certain embodiments, each cluster of genes comprises at least 3 genes. In certain embodiments, each cluster of genes comprises at least 5 genes. In certain embodiments, each cluster of genes comprises at least 7 genes. In certain embodiments, each cluster of genes comprises at least 10 genes. In certain embodiments, each cluster of genes comprises at least 12 genes. In certain embodiments, each cluster of genes comprises at least 15 genes. In certain embodiments, each cluster of genes comprises at least 20 genes.

In certain embodiments, a nucleic acid sample is extracted from a subject. In certain embodiments, the expression level of multiple genes in the tumor biopsy sample is determined by quantitating nucleic acids levels of the multiple genes using a DNA microarray.

In certain embodiments, at least one of the metagenes shares at least 3 of its defining genes in common with metagene 1, 2, 3, 4, 5, 6, or 7. In certain embodiments, at least one of the metagenes shares at least 50% of its defining genes in common with metagene 1, 2, 3, 4, 5, 6, or 7. In certain embodiments, at least one of the metagenes shares at least 75% of its defining genes in common with metagene 1, 2, 3, 4, 5, 6, or 7. In certain embodiments, at least one of the metagenes shares at least 90% of its defining genes in common with metagene 1, 2, 3, 4, 5, 6, or 7. In certain embodiments, at least one of the metagenes shares at least 95% of its defining genes in common with metagene 1, 2, 3, 4, 5, 6, or 7. In certain embodiments, at least one of the metagenes shares at least 98% of its defining genes in common with metagene 1, 2, 3, 4, 5, 6, or 7.

In certain embodiments, the cluster of genes for at least two of the metagenes share at least 50% of their genes in common with one of metagenes 1, 2, 3, 4, 5, 6, or 7. In certain embodiments, the cluster of genes for at least two of the metagenes share at least 75% of their genes in common with one of metagenes 1, 2, 3, 4, 5, 6, or 7. In certain embodiments, the cluster of genes for at least two of the metagenes share at least 90% of their genes in common with one of metagenes 1, 2, 3, 4, 5, 6, or 7. In certain embodiments, the cluster of genes for at least two of the metagenes share at least 95% of their genes in common with one of metagenes 1, 2, 3, 4, 5, 6, or 7. In certain embodiments, the cluster of genes for at least two of the metagenes share at least 98% of their genes in common with one of metagenes 1, 2, 3, 4, 5, 6, or 7.

In certain embodiments, the cluster of genes comprises at least 3 genes. In certain embodiments, the cluster of genes comprises at least 5 genes. In certain embodiments, the cluster of genes comprises at least 10 genes. In certain embodiments, the cluster of genes comprises at least 15 genes. In certain embodiments, the correlation-based clustering is Markov chain correlation-based clustering or K-means clustering.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show a gene expression signature that predicts sensitivity to docetaxel. (A) Strategy for generation of the chemotherapeutic response predictor. (B) Top panel—Cell lines from the NCI-60 panel used to develop the in vitro signature of docetaxel sensitivity. The figure shows a statistically significant difference (Mann Whitney U test of significance) in the IC₅₀/GI₅₀ and LC₅₀ of the cell lines chosen to represent the sensitive and resistant subsets. Bottom Panel—Expression plots for genes selected for discriminating the docetaxel resistant and sensitive NCI-60 cell lines, depicted by color coding with blue representing the lowest level and red the highest. Each column in the figure represents individual samples. Each row represents an individual gene, ordered from top to bottom according to regression coefficients. (C) Top Panel—Validation of the docetaxel response prediction model in an independent set of lung and ovarian cancer cell line samples. A collection of lung and ovarian cell lines were used in a cell proliferation assay to determine the 50% inhibitory concentration (IC₅₀) of docetaxel in the individual cell lines. A linear regression analysis demonstrates a statistically significant (p<0.01, log rank) relationship between the IC₅₀ of docetaxel and the predicted probability of sensitivity to docetaxel. Bottom panel—Validation of the docetaxel response prediction model in another independent set of 29 lung cancer cell line samples (Gemma A, Geo accession number: GSE 4127). A linear regression analysis demonstrates a very significant (p<0.001, log rank) relationship between the IC₅₀ of docetaxel and the predicted probability of sensitivity to docetaxel. (D) Left Panel—A strategy for assessment of the docetaxel response predictor as a function of clinical response in the breast neoadjuvant setting. Middle panel—Predicted probability of docetaxel sensitivity in a collection of samples from a breast cancer single agent neoadjuvant study. Twenty of twenty four samples (91.6%) were predicted accurately using the cell line based predictor of response to docetaxel. Right panel—A single variable scatter plot demonstrating a significance test of the predicted probabilities of sensitivity to docetaxel in the sensitive and resistant tumors (p<0.001, Mann Whitney U test of significance). (E) Left Panel—A strategy for assessment of the docetaxel response predictor as a function of clinical response in advanced ovarian cancer. Middle panel—Predicted probability of docetaxel sensitivity in a collection of samples from a prospective single agent salvage therapy study. Twelve of fourteen samples (85.7%) were predicted accurately using the cell line based predictor of response to docetaxel. Right panel—A single variable scatter plot demonstrating statistical significance (p<0.01, Mann Whitney U test of significance).

FIGS. 2A-2C show the development of a panel of gene expression signatures that predict sensitivity to chemotherapeutic drugs. (A) Gene expression patterns selected for predicting response to the indicated drugs. The genes involved the individual predictors are shown in Table 1. (B) Independent validation of the chemotherapy response predictors in an independent set of cancer cell lines³⁷ that have dose response and Affymetrix expression data.³⁸ A single variable scatter plot demonstrating a significance test of the predicted probabilities of sensitivity to any given drug in the sensitive and resistant cell lines (p value, Mann Whitney U test of significance). Red symbols indicate resistant cell lines, and blue symbols indicate those that are sensitive. (C) Prediction of single agent therapy response in patient samples using in vitro cell line based expression signatures of chemosensitivity. In each case, red represents non-responders (resistance) and blue represents responders (sensitivity). The left panel shows the predicted probability of sensitivity to topotecan when compared to actual clinical response data (n=48), the middle panel demonstrates the accuracy of the adriamycin predictor in a cohort of 122 samples (Evans W, GSE650 and GSE651). The right panel shows the predictive accuracy of the cell line based paclitaxel predictor when used as a salvage chemotherapy in advanced ovarian cancer (n=35). The positive and negative predictive values for all the predictors are summarized in Table 2.

FIGS. 3A-3B show the prediction of response to combination therapy. (A) Left Panel—Strategy for assessment of chemotherapy response predictors in combination therapy as a function of pathologic response. Middle panel—Prediction of patient response to neoadjuvant chemotherapy involving paclitaxel, 5-fluorouracil (5-FU), adriamycin, and cyclophosphamide (TFAC) using the single agent in vitro chemosensitivity signatures developed for each of these drugs. Right Panel—Prediction of response (38 non-responders, 13 responders) employing a combined probability predictor assessing the probability of all four chemosensitivity signatures in 51 patients treated with TFAC chemotherapy shows statistical significance (p<0.0001, Mann Whitney) between responders (blue) and non-responders (red). Response was defined as a complete pathologic response after completion of TFAC neoadjuvant therapy. (B) Left Panel—Prediction of patient response (n=45) to adjuvant chemotherapy involving 5-FU, adriamycin, and cyclophosphamide (FAC) using the single agent in vitro chemosensitivity predictors developed for these drugs. Middle panel—Prediction of response (34 responders, 11 non responders) employing a combined probability predictor assessing the probability of all four chemosensitivity signatures in 45 patients treated with FAC chemotherapy. Right panel—Kaplan Meier survival analysis for patients predicted to be sensitive (blue curve) or resistant (red curve) to FAC adjuvant chemotherapy.

FIG. 4 shows patterns of predicted sensitivity to common chemotherapeutic drugs in human cancers. Hierarchical clustering of a collection of breast (n=171), lung cancer (n=91) and ovarian cancer (n=119) samples according to patterns of predicted sensitivity to the various chemotherapeutics. These predictions were then plotted as a heatmap in which high probability of sensitivity/response is indicated by red, and low probability or resistance is indicated by blue.

FIGS. 5A-5B show the relationship between predicted chemotherapeutic sensitivity and oncogenic pathway deregulation. (A) Left Panel—Probability of oncogenic pathway deregulation as a function of predicted docetaxel sensitivity in a series of lung cancer cell lines (red=sensitive, blue=resistant). Right panel—Probability of oncogenic pathway deregulation as a function of predicted topotecan sensitivity in a series of ovarian cancer cell lines (red=sensitive, blue=resistant). (B) Left Panel—The lung cancer cell lines showing an increased probability of PI3 kinase were also more likely to respond to a PI3 kinase inhibitor (LY-294002) (p=0.001, log-rank test)), as measured by sensitivity to the drug in assays of cell proliferation. Further, those cell lines predicted to be resistant to docetaxel were more likely to be sensitive to PI3 kinase inhibition (p<0.001, log-rant test) Right panel—The relationship between Src pathway deregulation and topotecan resistance can be demonstrated in a set of 13 ovarian cancer cell lines. Ovarian cell lines that are predicted to be topotecan resistant have a higher likelihood of Src pathway deregulation and there is a significant linear relationship (p=0.001, log rank) between the probability of topotecan resistance and sensitivity to a drug that inhibits the Src pathway (SU6656).

FIG. 6 shows a scheme for utilization of chemotherapeutic and oncogenic pathway predictors for identification of individualized therapeutic options.

FIGS. 7A-7C show a patient-derived docetaxel gene expression signature predicts response to docetaxel in cancer cell lines. (A) Top panel—A ROC curve analysis to show the approach used to define a cut-off, using docetaxel as an example. Middle panel—A t-test plot of significance between the probability of docetaxel sensitivity and IC 50 for docetaxel sensitive in cell lines, shown by histologic type. Bottom panel—A linear regression analysis showing the significant correlation between predicted intro sensitivity and actual sensitivity (IC50 for docetaxel), in lung and ovarian cancer cell lines. (B) Generation of a docetaxel response predictor based on patient data that was then validated in a leave on out cross validation and linear regression analyses (p-value obtained by log-rank), evaluated against the IC₅₀ for docetaxel in two NCI-60 cell line drug screening experiments. (C) A comparison of predictive accuracies between a predictor for docetaxel generated from the cell line data (left panel, accuracy: 85.7%) and a predictor generated from patients treatment data (right panel, accuracy: 64.3%) shows the relative inferiority of the latter approach, when applied to an independent dataset of ovarian cancer patients treated with single agent docetaxel.

FIGS. 8A-8C show the development of gene expression signatures that predict sensitivity to a panel of commonly used chemotherapeutic drugs. Panel A shows the gene expression models selected for predicting response to the indicated drugs, with resistant lines on the left, sensitive on the right for each predictor. Panel B shows the leave one out cross validation accuracy of the individual predictors. Panel C demonstrates the results of an independent validation of the chemotherapy response predictors in an independent set of cancer cell lines³⁷ shown as a plot with error bars (blue—sensitive, red—resistant).

FIG. 9 shows the specificity of chemotherapy response predictors. In each case, individual predictors of response to the various cytotoxic drugs was plotted against cell lines known to be sensitive or sensitive to a given chemotherapeutic agent (e.g., adriamycin, paclitaxel).

FIG. 10A-10C shows the absolute probabilities of response to various chemotherapies in human lung and breast cancer samples.

FIG. 11 shows the relationships in predicted probability of response to chemotherapies in breast and lung. In each case, a regression analysis (log rank) of predicted probability of response of two drugs is shown.

FIG. 12 shows a gene expression based signature of PI3 kinase pathway deregulation. Image intensity display of expression levels for genes that most differentiate control cells expressing GFP from cells expressing the oncogenic activity of PI3 kinase. The expression value of genes composing each signature is indicated by color, with blue representing the lowest value and red representing the highest level. The panel below shows the results of a leave one out cross validation showing a reliable differentiation between GFP controls (blue) and cells expressing PI3 kinase (red).

FIGS. 13A-13C show the relationship between oncogenic pathway deregulation and chemosensitivity patterns (using docetaxel as an example). (A) Probability of oncogenic pathway deregulation as a function of predicted docetaxel sensitivity in the NCI-60 cell line panel (red=sensitive, blue=resistant). (B) Linear regression analysis (log-rank test of significance) to identify relationships between predicted docetaxel sensitivity or resistance and deregulation of PI3 kinase, E2F3, and Src pathways. (C) A non-parametric t-test of significance demonstrating a significant difference in docetaxel sensitivity, between those cell lines predicted to be either pathway deregulated (>50% probability, red) or quiescent (<50% probability, blue), shown for both E2F and PI3 kinase pathways.

FIG. 14 shows a scatter plot showing a linear regression analysis that identifies a statistically significant correlation between probability of docetaxel resistance and PI3 Kinase pathway activation in an independent cohort of 17 non-small cell lung cancer cell lines.

FIG. 15 shows a functional block diagram of general purpose computer system 1500 for performing the functions of the software provided by the invention.

### BRIEF DESCRIPTION OF THE TABLES

Table 1 lists the predictor set for commonly used chemotherapeutics.

Table 2 is a summary of the chemotherapy response predictors—validations in cell line and patient data sets.

Table 3 shows an enrichment analysis shows that a genomic-guided response prediction increases the probability of a clinical response in the different data sets studied.

Table 4 shows the accuracy of genomic-based chemotherapy response predictors is compared to previously reported predictors of response.

Table 5 lists the genes that constitute the predictor of PI3 kinase activation.

### DETAILED DESCRIPTION OF THE INVENTION

An individual who has cancer frequently has progressed to an advanced stage before any symptoms appear. The difficulty with administering one or more chemotherapeutic agents is that not all individuals with cancer will respond favorably to the chemotherapeutic agent selected by the physician. Frequently, the administration of one or more chemotherapeutic agents results in the individual becoming even more ill from the toxicity of the agent and the cancer still persists. Due to the cytotoxic nature of chemotherapeutic agents, the individual is physically weakened and his/her immunologically compromised system cannot generally tolerate multiple rounds of “trial and error” type of therapy. Hence a treatment plan that is personalized for the individual is highly desirable.

The inventors have described gene expression profiles associated with determining whether an individual afflicted with cancer will respond to a therapy, and in particular to a therapeutic agents such as salvage agents. This analysis has been coupled with gene expression signatures that reflect the deregulation of various oncogenic signaling pathways to identify unique characteristics of chemotherapeutic resistant cancers that can guide the use of these drugs in patients with chemotherapeutic resistant disease. The invention thus provides integrating gene expression profiles that predict chemotherapeutic response and oncogenic pathway status as a strategy for developing personalized treatment plans for individual patients.

### DEFINITIONS

“Platinum-based therapy” and “platinum-based chemotherapy” are used interchangeably herein and refers to agents or compounds that are associated with platinum.

As used herein, “array” and “microarray” are interchangeable and refer to an arrangement of a collection of nucleotide sequences in a centralized location. Arrays can be on a solid substrate, such as a glass slide, or on a semi-solid substrate, such as nitrocellulose membrane. The nucleotide sequences can be DNA, RNA, or any permutations thereof. The nucleotide sequences can also be partial sequences from a gene, primers, whole gene sequences, non-coding sequences, coding sequences, published sequences, known sequences, or novel sequences.

A “complete response” (CR) is defined as a complete disappearance of all measurable and assessable disease or, in the absence of measurable lesions, a normalization of the CA-125 level following adjuvant therapy. An individual who exhibits a complete response is known as a “complete responder.”

An “incomplete response” (IR) includes those who exhibited a “partial response” (PR), had “stable disease” (SD), or demonstrated “progressive disease” (PD) during primary therapy.

A “partial response” refers to a response that displays 50% or greater reduction in the product obtained from measurement of each bi-dimensional lesion for at least 4 weeks or a drop in the CA-125 by at least 50% for at least 4 weeks.

“Progressive disease” refers to response that is a 50% or greater increase in the product from any lesion documented within 8 weeks of initiation of therapy, the appearance of any new lesion within 8 weeks of initiation of therapy, or any increase in the CA-125 from baseline at initiation of therapy.

“Stable disease” was defined as disease not meeting any of the above criteria.

“Effective amount” refers to an amount of a chemotherapeutic agent that is sufficient to exert a biological effect in the individual. In most cases, an effective amount has been established by several rounds of testing for submission to the FDA. It is desirable for an effective amount to be an amount sufficient to exert cytotoxic effects on cancerous cells.

“Predicting” and “prediction” as used herein does not mean that the event will happen with 100% certainty. Instead it is intended to mean the event will more likely than not happen.

As used herein, “individual” and “subject” are interchangeable. A “patient” refers to an “individual” who is under the care of a treating physician. In one embodiment, the subject is a male. In one embodiment, the subject is a female.

### General Techniques

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning. A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies. A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000) and Casarett and Doull's Toxicology The Basic Science of Poisons, C. Klaassen, ed., 6th edition (2001).

### Methods of Predicting Responsivity to Salvage Agents

Gene expression profiles may be obtained from tumor samples taken during surgery to debulk individuals with ovarian cancer. It is also possible to generate a predictor set for predicting responsivity to common chemotherapy agents by using publicly available data. Numerous websites exist that share data obtained from microarray analysis. In one embodiment, gene expression profiling data obtained from analysis of 60 cancerous cells lines, known herein as NCI-60, can be used to generate a training set for predicting responsivity to cancer therapy agents. The NCI-60 training set can be validated by the same type of “Leave-one-out” cross-validation as described earlier.

The predictor sets for the other salvage therapy agents are shown in Table 1. The genes listed in Table 1 represent, to the best of Applicants' knowledge, a novel gene predictor set. The genes in the predictor set would not have been obvious to one of ordinary skill in the art. These predictor sets are used as a reference set to compare the first gene expression profile from an individual with ovarian cancer to determine if she will be responsive to a particular salvage agent. In certain embodiments, the methods of the application are performed outside of the human body.

Method of Treating Individuals with Ovarian Cancer

This methods described herein also include treating an individual afflicted with ovarian cancer. In the instance where the individual is predicted to be a non-responder to platinum-based therapy, a physician may decide to administer salvage therapy agent alone. In most instances, the treatment will comprise a combination of a platinum-based therapy and a salvage agent. In one embodiment, the treatment will comprise a combination of a platinum-based therapy and an inhibitor of a signal transduction pathway that is deregulated in the individual with ovarian cancer.

In one embodiment, the platinum-based therapy and a salvage agent are administered in an effective amount concurrently. In another embodiment, the platinum-based therapy and a salvage agent are administered in an effective amount in a sequential manner. In yet another embodiment, the salvage therapy agent is administered in an effective amount by itself. In yet another embodiment, the salvage therapy agent is administered in an effective amount first and then followed concurrently or step-wise by a platinum-based therapy.

Methods of Predicting/Estimating the Efficacy of a Therapeutic Agent in Treating a Individual Afflicted with Cancer

One aspect of the invention provides a method for predicting, estimating, aiding in the prediction of, or aiding in the estimation of, the efficacy of a therapeutic agent in treating a subject afflicted with cancer. In certain embodiments, the methods of the application are performed outside of the human body.

One method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more statistical tree models applied to the values of the metagenes, wherein each model includes one or more nodes, each node representing a metagene, each node including a statistical predictive probability of tumor sensitivity to the therapeutic agent, wherein at least one of the metagenes comprises at least 3 genes in metagenes 1, 2, 3, 4, 5, 6, or 7, thereby estimating the efficacy of a therapeutic agent in a subject afflicted with cancer. Another method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a cluster of genes associated tumor sensitivity to the therapeutic agent; and (c) averaging the predictions of one or more binary regression models applied to the values of the metagenes, wherein each model includes a statistical predictive probability of tumor sensitivity to the therapeutic agent, wherein at least one of the metagenes comprises at least 3 genes in metagenes 1, 2, 3, 4, 5, 6, or 7, thereby estimating the efficacy of a therapeutic agent in a subject afflicted with cancer.

In one embodiment, the predictive methods of the invention predict the efficacy of a therapeutic agent in treating a subject afflicted with cancer with at least 70% accuracy. In another embodiment, the methods predict the efficacy of a therapeutic agent in treating a subject afflicted with cancer with at least 80% accuracy. In another embodiment, the methods predict the efficacy of a therapeutic agent in treating a subject afflicted with cancer with at least 85% accuracy. In another embodiment, the methods predict the efficacy of a therapeutic agent in treating a subject afflicted with cancer with at least 90% accuracy. In another embodiment, the methods predict the efficacy of a therapeutic agent in treating a subject afflicted with cancer with at least 70%, 80%, 85% or 90% accuracy when tested against a validation sample. In another embodiment, the methods predict the efficacy of a therapeutic agent in treating a subject afflicted with cancer with at least 70%, 80%, 85% or 90% accuracy when tested against a set of training samples. In another embodiment, the methods predict the efficacy of a therapeutic agent in treating a subject afflicted with cancer with at least 70%, 80%, 85% or 90% accuracy when tested on human primary tumors ex vivo or in vivo.

### (A) Tumor Sample

In one embodiment, the predictive methods of the invention comprise determining the expression level of genes in a tumor sample from the subject, preferably a breast tumor, an ovarian tumor, and a lung tumor. In one embodiment, the tumor is not a breast tumor. In one embodiment, the tumor is not an ovarian tumor. In one embodiment, the tumor is not a lung tumor. In one embodiment of the methods described herein, the methods comprise the step of surgically removing a tumor sample from the subject, obtaining a tumor sample from the subject, or providing a tumor sample from the subject. In one embodiment, the sample contains at least 40%, 50%, 60%, 70%, 80% or 90% tumor cells. In preferred embodiments, samples having greater than 50% tumor cell content are used. In one embodiment, the tumor sample is a live tumor sample. In another embodiment, the tumor sample is a frozen sample. In one embodiment, the sample is one that was frozen within less than 5, 4, 3, 2, 1, 0.75, 0.5, 0.25, 0.1, 0.05 or less hours after extraction from the patient. Preferred frozen sample include those stored in liquid nitrogen or at a temperature of about −80 C or below.

### (B) Gene Expression

The expression of the genes may be determined using any methods known in the art for assaying gene expression. Gene expression may be determined by measuring mRNA or protein levels for the genes. In a preferred embodiment, an mRNA transcript of a gene may be detected for determining the expression level of the gene. Based on the sequence information provided by the GenBank™ database entries, the genes can be detected and expression levels measured using techniques well known to one of ordinary skill in the art. For example, sequences within the sequence database entries corresponding to polynucleotides of the genes can be used to construct probes for detecting mRNAs by, e.g., Northern blot hybridization analyses. The hybridization of the probe to a gene transcript in a subject biological sample can be also carried out on a DNA array. The use of an array is preferable for detecting the expression level of a plurality of the genes. As another example, the sequences can be used to construct primers for specifically amplifying the polynucleotides in, e.g., amplification-based detection methods such as reverse-transcription based polymerase chain reaction (RT-PCR). Furthermore, the expression level of the genes can be analyzed based on the biological activity or quantity of proteins encoded by the genes.

Methods for determining the quantity of the protein includes immunoassay methods. Paragraphs 98-123 of U.S. Patent Pub No. 2006-0110753 provide exemplary methods for determining gene expression. Additional technology is described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; as well as WO 95/21265; WO 96/31622; WO 97/10365; WO 97/27317; EP 373 203; and EP 785 280.

In one exemplary embodiment, about 1-50 mg of cancer tissue is added to a chilled tissue pulverizer, such as to a BioPulverizer H tube (Bio101 Systems, Carlsbad, Calif.). Lysis buffer, such as from the Qiagen Rneasy Mini kit, is added to the tissue and homogenized. Devices such as a Mini-Beadbeater (Biospec Products, Bartlesville, Okla.) may be used. Tubes may be spun briefly as needed to pellet the garnet mixture and reduce foam. The resulting lysate may be passed through syringes, such as a 21 gauge needle, to shear DNA. Total RNA may be extracted using commercially available kits, such as the Qiagen RNeasy Mini kit. The samples may be prepared and arrayed using Affymetrix U133 plus 2.0 GeneChips or Affymetrix U133A GeneChips.

In one embodiment, determining the expression level of multiple genes in a tumor sample from the subject comprises extracting a nucleic acid sample from the sample from the subject, preferably an mRNA sample. In one embodiment, the expression level of the nucleic acid is determined by hybridizing the nucleic acid, or amplification products thereof, to a DNA microarray. Amplification products may be generated, for example, with reverse transcription, optionally followed by PCR amplification of the products.

### (C) Genes Screened

In one embodiment, the predictive methods of the invention comprise determining the expression level of all the genes in the cluster that define at least one therapeutic sensitivity/resistance determinative metagene. In one embodiment, the predictive methods of the invention comprise determining the expression level of at least 50%, 60%, 70%, 80%, 90%, 95%, 98%, 99% of the genes in each of the clusters that defines 1, 2, 3, 4 or 5 or more of metagenes 1, 2, 3, 4, 5, 6 and 7.

In one embodiment, at least 50%, 60%, 70%, 80%, 90%, 95%, 98%, 99% of the genes whose expression levels are determined to predict 5-FU sensitivity (or the genes in the cluster that define a metagene having said predictivity) are genes represented by the following symbols: LOC92755 (TUBB, LOC648765), CDKN2A, TRA@, GABRA3, COL1A2, ACTB, PDLIM4, ACTA2, FTSJ1, NBR1 (LOC727732), CFL1, ATP1A2, APOC4, KIAA1509, ZNF516, GRIK5, PDE5A, ARSF, ZC3H7B, WBP4, CSTB, TSPY1 (TSPY2, LOC653174, LOC728132, LOC728137, LOC728395, LOC728403, LOC728412), HTR2B, KBTBD11, SLC25A17, HMGN3, FIBP, IFT140, FAM63B, ZNF337, KIAA0100, FAM13C1, STK25, CPNE1, PEX19, EIF5B, EEF1A1 (APOLD1, LOC440595), SRR, THEM2, ID4, GGT1 (GGTL4), IFNA10, TUBB2A (TUBB4, TUBB2B), and TUBB3.

In one embodiment, at least 50%, 60%, 70%, 80%, 90%, 95%, 98%, 99% of the genes whose expression levels are determined to predict adriamycin sensitivity are genes represented by the following symbols: MLANA, CSPG4, DDR2, ETS2, EGFR, BIK, CD24, ZNF185, DSCR1, GSN, TPST1, LCN2, FAIM3, NCK2, PDZRN3, FKBP2, KRT8, NRP2, PKP2, CLDN3, CAPN1, STXBP1, LY96, WWC1, C10orf56, SPINT2, MAGED2, SYNGR2, SGCD, LAMC2, C19orf21, ZFHX1B, KRT18, CYBA, DSP, ID1, ID1, PSAP, ZNF629, ARHGAP29, ARHGAP8 (LOC553158), GPM6B, EGFR, CALU, KCNK1, RNF144, FEZ1, MEST, KLF5, CSPG4, FLNB, GYPC, SLC23A2, MITF, PITPNM1, GPNMB, PMP22, PLXNB3 (SRPK3), MIA, RAB40C, MAD2L1BP, PLOD3, VIL2, KLF9, PODXL, ATP6V1B2, SLC6A8, PLP1, KRT7, PKP3, DLG3, ZHX2, LAMA5, SASH1, GAS1, TACSTD1, GAS1, and CYP27A1.

In one embodiment, at least 50%, 60%, 70%, 80%, 90%, 95%, 98%, 99% of the genes whose expression levels are determined to predict cytoxan sensitivity (or the genes in the cluster that define a metagene having said predictivity) are genes represented by the following symbols: DAP3, RPS9, TTR, ACTB, MARCKS, GGT1 (GGT2), GGTL4, GGTLA4, LOC643171, LOC653590, LOC728226, LOC728441, LOC729838, LOC731629), FANCA, CDC42EP3, TSPAN4, C6orf145, ARNT2, KIF22 (LOC728037), NBEAL2, CAV1, SCRN1, SCHIP1, PHLDB1, AKAP12, ST5, SNAI2, ESD, ANP32B, CD59, ACTN1, CD59, PEG10, SMARCA1, GGCX, SAMD4A, CNN3, LPP, SNRPF, SGCE, CALD1, and C22orf5.

In one embodiment, at least 50%, 60%, 70%, 80%, 90%, 95%, 98%, 99% of the genes whose expression levels are determined to predict docetaxel sensitivity (or the genes in the cluster that define a metagene having said predictivity) are genes represented by the following symbols: BLR1, EIF4A2, FLT1, BAD, PIP5K₃, BIN1, YBX1, BCKDK, DOHH, FOXD1, TEX261, NBR1 (LOC727732), APOA4, DDX5, TBCA, USP52, SLC25A36, CHP, ANKRD28, PDXK, ATP6AP1, SETD2, CCS, BRD2, ASPHD1, B4GALT6, ASL, CAPZA2, STARD3, LIMK2 (PPP1R14BP1), BANF1, GNB2, ENSA, SH3GL1, ACVR1B, SLC6A1, PPP2R1A, PCGF1, LOC643641, INPP5A, TLE1, PLLP, ZKSCAN1, TIAL1, TK1, PPP2R1A, and PSMB6.

In one embodiment, at least 50%, 60%, 70%, 80%, 90%, 95%, 98%, 99% of the genes whose expression levels are determined to predict etoposide sensitivity are genes represented by the following symbols: LIMK1, LIG3, AXL, IFI16, MMP14, GRB7, VAV2, FLT1, JUP, FN1, FN1, PKM2, LYPLA3, RFTN1, LAD1, SPINT1, CLDN3, PTRF, SPINT2, MMP14, FAAH, CLDN4, ST14, C19orf21, KIAA0506, LLGL2 (MADD), COBL, ZFHX1B, GBP1, IER2, PPL, TMEM30B, CNKSR1, CLDN7, BTN3A2, BTN3A2, TUBB2A, MAP7, HNRNPG-T, UGCG, GAK, PKP3, DFNA5, DAB2, TACSTD1, SPARC, and PPP2R5A.

In one embodiment, at least 50%, 60%, 70%, 80%, 90%, 95%, 98%, 99% of the genes whose expression levels are determined to predict taxol sensitivity (or the genes in the cluster that define a metagene having said predictivity) are genes represented by the following symbols: NR2F6, TOP2B, RARG, PCNA, PTPN11, ATM, NFATC4, CACNG1, C22orf31, PIK3R2, PRSS12, MYH8, SCCPDH, PHTF2, IQSEC2, TRPC3, TRAFD1, HEPH, SOX30, GATM, LMNA, HD, YIPF3, DNPEP, PCDH9, KLHDC3, SLC10A3, LHX2, CKS2, SECTM1, SF1, RPS6KA4, DYRK2, GDI2, and IFI30.

In one embodiment, at least 50%, 60%, 70%, 80%, 90%, 95%, 98%, 99% of the genes whose expression levels are determined to predict topotecan sensitivity (or the genes in the cluster that define a metagene having said predictivity) are genes represented by the following symbols: DUSP1, THBS1, AXL, RAP1GAP, QSCN6, IL1R1, TGFBI, PTX3, BLM, TNFRSF1A, FGF2, VEGFC, ACO2, FARSLA, RIN2, FGF2, RRAS, FIGF, MYB, CDH2, FGFR1, FGFR1, LAMC1, HIST1H4K (HIST1H4J), COL6A2, TMC6, PEA15, MARCKS, CKAP4, GJA1, FBN1, BASP1, BASP1, BTN2A1, ITGB1, DKFZP686A01247, MYLK, LOXL2, HEG1, DEGS1, CAP2, CAP2, PTGER4, BAI2, NUAK1, DLEU1 (SPANXC), RAB11FIP5, FSTL3, MYL6, VIM, GNA12, PRAF2, PTRF, CCL2, PLOD2, COL6A2, ATP5G3, GSR, NDUFS3, ST14, NID1, MYO1D, SDHB, CAV1, DPYSL3, PTRF, FBXL2, RIN2, PLEKHC1, CTGF, COL4A2, TPM1, TPM1, TPM1, FZD2, LOXL1, SYK, HADHA, TNFAIP1, NNMT, HPGD, MRC2, MEIS3P1, AOX1, SEMA3C, SEMA3C, SYNE1, SERPINE1, IL6, RRAS, GPD1L, AXL, WDR23, CLDN7, IL15, TNFAIP2, CYR61, LRP1, AMOTL2, PDE1B, SPOCK1, RA114, PXDN, COL4A1, CIR, KIAA0802 (C21orf57), C5orf13, TUFM, EDIL3, BDNF, PRSS23, ATP5A1, FRAT2, C16orf51, TUSC4, NUP50, TUBA3, NFIB, TLE4, AKT3, CRIM1, RAD23A, COX5A, SMCR7L, MXRA7, STARD7, STC1, TTC28, PLK2, TGDS, CALD1, OPTN, IFITM3, DFNA5, FGFR1, HTATIP, SYK, LAMB1, FZD2, SERPINE1, THBS1, CCL2, ITGA3, ITGA3, and UBE2A.

Table 1 shows the genes in the cluster that define metagenes 1-7 and indicates the therapeutic agent whose sensitivity it predicts. In one embodiment, at least 3, 5, 7, 9, 10, 12, 14, 16, 18, 20, 25, 30, 40 or 50 genes in the cluster of genes defining a metagene used in the methods described herein are common to metagene 1, 2, 3, 4, 5, 6 or 7, or to combinations thereof.

### (D) Metagene Valuation

In one embodiment, the predictive methods of the invention comprise defining the value of one or more metagenes from the expression levels of the genes. A metagene value is defined by extracting a single dominant value from a cluster of genes associated with sensitivity to an anti-cancer agent, preferably an anti-cancer agent such as docetaxel, paclitaxel, topotecan, adriamycin, etoposide, fluorouracil (5-FU), and cyclophosphamide. In one embodiment, the agent is selected from alkylating agents (e.g., nitrogen mustards), antimetabolites (e.g., pyrimidine analogs), radioactive isotopes (e.g., phosphorous and iodine), miscellaneous agents (e.g., substituted ureas) and natural products (e.g., vinca alkyloids and antibiotics). In another embodiment, the therapeutic agent is selected from the group consisting of allopurinol sodium, dolasetron mesylate, pamidronate disodium, etidronate, fluconazole, epoetin alfa, levamisole HCL, amifostine, granisetron HCL, leucovorin calcium, sargramostim, dronabinol, mesna, filgrastim, pilocarpine HCL, octreotide acetate, dexrazoxane, ondansetron HCL, ondansetron, busulfan, carboplatin, cisplatin, thiotepa, melphalan HCL, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorucbicin citrate, idarubicin HCL, plimycin, mitomycin, pentostatin, mitoxantrone, valrubicin, cytarabine, fludarabine phosphate, floxuridine, cladribine, methotrexate, mercaptipurine, thioguanine, capecitabine, methyltestosterone, nilutamide, testolactone, bicalutamide, flutamide, anastrozole, toremifene citrate, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, altretamine, topotecan HCL, hydroxyurea, interferon alpha-2b, mitotane, procarbazine HCL, vinorelbine tartrate, E. coli L-asparaginase, Erwinia L-asparaginase, vincristine sulfate, denileukin diftitox, aldesleukin, rituximab, interferon alpha-2a, paclitaxel, docetaxel, BCG live (intravesical), vinblastine sulfate, etoposide, tretinoin, teniposide, porfimer sodium, fluorouracil, betamethasone sodium phosphate and betamethasone acetate, letrozole, etoposide citrororum factor, folinic acid, calcium leucouorin, 5-fluorouricil, adriamycin, cytoxan, and diamino-dichloro-platinum.

In a preferred embodiment, the dominant single value is obtained using single value decomposition (SVD). In one embodiment, the cluster of genes of each metagene or at least of one metagene comprises at least 3, 4, 5, 6, 7, 8, 9, 10, 12, 15, 18, 20 or 25 genes. In one embodiment, the predictive methods of the invention comprise defining the value of 2, 3, 4, 5, 6, 7, 8, 9 or 10 or more metagenes from the expression levels of the genes.

In preferred embodiments of the methods described herein, at least 1, 2, 3, 4, 5, 6, 7, 8 or 9 of the metagenes is metagene 1, 2, 3, 4, 5, 6, or 7. In one embodiment, at least one of the metagenes comprises 3, 4, 5, 6, 7, 8, 9 or 10 or more genes in common with any one of metagenes 1, 2, 3, 4, 5, 6, or 7. In one embodiment, a metagene shares at least 50%, 60%, 70%, 80%, 90%, 95%, 98%, 99% of the genes in its cluster in common with a metagene selected from 1, 2, 3, 4, 5, 6, or 7.

In one embodiment, the predictive methods of the invention comprise defining the value of 2, 3, 4, 5, 6, 7, 8 or more metagenes from the expression levels of the genes. In one embodiment, the cluster of genes from which any one metagene is defined comprises at least 3, 4, 5, 6, 7, 8, 9, 10, 11, 12, 13, 14, 15, 16, 17, 18, 19, 20, 22 or 25 genes.

In one embodiment, the predictive methods of the invention comprise defining the value of at least one metagene wherein the genes in the cluster of genes from which the metagene is defined, shares at least 50%, 60%, 70%, 80%, 90%, 95% or 98% of genes in common to any one of metagenes 1, 2, 3, 4, 5, 6, or 7. In one embodiment, the predictive methods of the invention comprise defining the value of at least two metagenes, wherein the genes in the cluster of genes from which each metagene is defined share at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of at least three metagenes, wherein the genes in the cluster of genes from which each metagene is defined shares at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of at least four metagenes, wherein the genes in the cluster of genes from which each metagene is defined shares at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of at least five metagenes, wherein the genes in the cluster of genes from which each metagene is defined shares at least 50%, 60%, 70%, 80%, 90%, 95% or 98% of genes in common to anyone of metagenes 1, 2, 3, 4, 5, 6, or 7. In one embodiment, the predictive methods of the invention comprise defining the value of a metagene from a cluster of genes, wherein at least 2, 3, 4, 5, 6, 7, 8, 9, 10, 11, 12, 13, 14, 15, 16, 17, 18, 19 or 20 genes in the cluster are selected from the genes listed in Table 1.

In one embodiment, at least one of the metagenes is metagene 1, 2, 3, 4, 5, 6, or 7. In one embodiment, at least two of the metagenes are selected from metagenes 1, 2, 3, 4, 5, 6, or 7. In one embodiment, at least three of the metagenes are selected from metagenes 1, 2, 3, 4, 5, 6, or 7. In one embodiment, at least three of the metagenes are selected from metagenes 1, 2, 3, 4, 5, 6, or 7. In one embodiment, at least four of the metagenes are selected from metagenes 1, 2, 3, 4, 5, 6, or 7. In one embodiment, at least five or more of the metagenes are selected from metagenes 1, 2, 3, 4, 5, 6, or 7. In one embodiment of the methods described herein, one of the metagenes whose value is defined (i) is metagene 1 or (ii) shares at least 2, 3, 4, 5, 6, 7, 8, 9, 10, 11, 12 or 13 genes in common with metagene 1. In one embodiment of the methods described herein, one of the metagenes whose value is defined (i) is metagene 2 or (ii) shares at least 2, 3, 4, 5, 6, 7, 8, 9, 10, 11 or 12 genes in common with metagene 2. In one embodiment of the methods described herein, one of the metagenes whose value is defined (i) is metagene 3 or (ii) shares at least 2, 3 or 4 genes in common with metagene 3. In one embodiment of the methods described herein, one of the metagenes whose value is defined (i) is metagene 4 or (ii) shares at least 2, 3, 4, 5, 6, 7, 8, 9, 10, 11, 12, 13, 14, 15, 16, 17, 18, 19, 20, 21, 22, 23, 24 or 25 genes in common with metagene 4. In one embodiment of the methods described herein, one of the metagenes whose value is defined (i) is metagene 5 or (ii) shares at least 2, 3, 4, 5, 6, 7, 8, 9, 10, 11, 12, 13, 14 or 15 genes in common with metagene 5. In one embodiment of the methods described herein, one of the metagenes whose value is defined (i) is metagene 6 or (ii) shares at least 2, 3, 4, 5, 6, 7, 8, 9, 10, 11, 12 or 13 genes in common with metagene 6. In one embodiment of the methods described herein, one of the metagenes whose value is defined (i) is metagene 7 or (ii) shares at least 2, 3, 4, 5, 6, 7, 8, 9 or 10 genes in common with metagene 7.

(E) Predictions from Tree Models

In one embodiment, the predictive methods of the invention comprise averaging the predictions of one or more statistical tree models applied to the metagenes values, wherein each model includes one or more nodes, each node representing a metagene, each node including a statistical predictive probability of sensitivity to an anti-cancer agent. The statistical tree models may be generated using the methods described herein for the generation of tree models. General methods of generating tree models may also be found in the art (See for example Pitman et al., Biostatistics 2004; 5:587-601; Denison et al. Biometrika 1999; 85:363-77; Nevins et al. Hum Mol Genet. 2003; 12:R153-7; Huang et al. Lancet 2003; 361:1590-6; West et al. Proc Natl Acad Sci USA 2001; 98:11462-7; U.S. Patent Pub. Nos. 2003-0224383; 2004-0083084; 2005-0170528; 2004-0106113; and U.S. application Ser. No. 11/198,782).

In one embodiment, the predictive methods of the invention comprise deriving a prediction from a single statistical tree model, wherein the model includes one or more nodes, each node representing a metagene, each node including a statistical predictive probability of sensitivity to an anti-cancer agent. In a preferred embodiment, the tree comprises at least 2 nodes. In a preferred embodiment, the tree comprises at least 3 nodes. In a preferred embodiment, the tree comprises at least 3 nodes. In a preferred embodiment, the tree comprises at least 4 nodes. In a preferred embodiment, the tree comprises at least 5 nodes.

In one embodiment, the predictive methods of the invention comprise averaging the predictions of one or more statistical tree models applied to the metagenes values, wherein each model includes one or more nodes, each node representing a metagene, each node including a statistical predictive probability of sensitivity to an anti-cancer agent. Accordingly, the invention provides methods that use mixed trees, where a tree may contain at least two nodes, where each node represents a metagene representative to the sensitivity/resistance to a particular agent.

In one embodiment, the statistical predictive probability is derived from a Bayesian analysis. In another embodiment, the Bayesian analysis includes a sequence of Bayes factor based tests of association to rank and select predictors that define a node binary split, the binary split including a predictor/threshold pair. Bayesian analysis is an approach to statistical analysis that is based on the Bayes law, which states that the posterior probability of a parameter p is proportional to the prior probability of parameter p multiplied by the likelihood of p derived from the data collected. This methodology represents an alternative to the traditional (or frequentist probability) approach: whereas the latter attempts to establish confidence intervals around parameters, and/or falsify a-priori null-hypotheses, the Bayesian approach attempts to keep track of how apriori expectations about some phenomenon of interest can be refined, and how observed data can be integrated with such a-priori beliefs, to arrive at updated posterior expectations about the phenomenon. Bayesian analysis have been applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” nodes using a splitting rule. For instance, for clarification, training data contains sample vectors that have one or more measurement variables and one variable that determines that class of the sample. Various splitting rules may be used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently, used purity functions are entropy, gini-index, and towing rule. A statistical predictive tree model to which Bayesian analysis is applied may consistently deliver accurate results with high predictive capabilities.

Gene expression signatures that reflect the activity of a given pathway may be identified using supervised classification methods of analysis previously described (e.g., West, M. et al. Proc Natl Acad Sci USA 98, 11462-11467, 2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models assign the relative probability of sensitivity to an anti-cancer agent.

In one embodiment, the methods for defining one or more statistical tree models predictive of cancer sensitivity to an anti-cancer agent comprise identifying clusters of genes associated with metastasis by applying correlation-based clustering to the expression level of the genes. In one embodiment, the clusters of genes that define each metagene are identified using supervised classification methods of analysis previously described. See, for example, West, M. et al. Proc Natl Acad Sci USA 98, 11462-11467 (2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models assign the relative probability of sensitivity to an anti-cancer agent.

In one embodiment, identification of the clusters comprises screening genes to reduce the number by eliminating genes that show limited variation across samples or that are evidently expressed at low levels that are not detectable at the resolution of the gene expression technology used to measure levels. This removes noise and reduces the dimension of the predictor variable. In one embodiment, identification of the clusters comprises clustering the genes using k-means, correlated-based clustering. Any standard statistical package may be used, such as the xcluster software created by Gavin Sherlock (http://genetics.stanford.edu/˜sherlock/cluster.html). A large number of clusters may be targeted so as to capture multiple, correlated patterns of variation across samples, and generally small numbers of genes within clusters. In one embodiment, identification of the clusters comprises extracting the dominant singular factor (principal component) from each of the resulting clusters. Again, any standard statistical or numerical software package may be used for this; this analysis uses the efficient, reduced singular value decomposition function. In one embodiment, the foregoing methods comprise defining one or more metagenes, wherein each metagene is defined by extracting a single dominant value using single value decomposition (SVD) from a cluster of genes associated with estimating the efficacy of a therapeutic agent in treating a subject afflicted with cancer.

In one embodiment, the methods for defining one or more statistical tree models predictive of cancer sensitivity to an anti-cancer agent comprise defining a statistical tree model, wherein the model includes one or more nodes, each node representing a metagene, each node including a statistical predictive probability of the efficacy of a therapeutic agent in treating a subject afflicted with cancer. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. Iterative out-of-sample, cross-validation predictions are then performed leaving each tumor out of the data set one at a time, refitting the model from the remaining tumors and using it to predict the hold-out case. This rigorously tests the predictive value of a model and mirrors the real-world prognostic context where prediction of new cases as they arise is the major goal.

In one embodiment, a formal Bayes' factor measure of association may be used in the generation of trees in a forward-selection process as implemented in traditional classification tree approaches. Consider a single tree and the data in a node that is a candidate for a binary split. Given the data in this node, one may construct a binary split based on a chosen (predictor, threshold) pair (χ, τ) by (a) finding the (predictor, threshold) combination that maximizes the Bayes' factor for a split, and (b) splitting if the resulting Bayes' factor is sufficiently large. By reference to a posterior probability scale with respect to a notional 50:50 prior, Bayes' factors of 2.2, 2.9, 3.7 and 5.3 correspond, approximately, to probabilities of 0.9, 0.95, 0.99 and 0.995, respectively. This guides the choice of threshold, which may be specified as a single value for each level of the tree. Bayes' factor thresholds of around 3 in a range of analyses may be used. Higher thresholds limit the growth of trees by ensuring a more stringent test for splits.

In one non-limiting exemplary embodiment of generating statistical tree models, prior to statistical modeling, gene expression data is filtered to exclude probe sets with signals present at background noise levels, and for probe sets that do not vary significantly across tumor samples. A metagene represents a group of genes that together exhibit a consistent pattern of expression in relation to an observable phenotype. Each signature summarizes its constituent genes as a single expression profile, and is here derived as the first principal component of that set of genes (the factor corresponding to the largest singular value) as determined by a singular value decomposition. Given a training set of expression vectors (of values across metagenes) representing two biological states, a binary probit regression model may be estimated using Bayesian methods. Applied to a separate validation data set, this leads to evaluations of predictive probabilities of each of the two states for each case in the validation set. When predicting sensitivity to an anti-cancer agent from an Tumor sample, gene selection and identification is based on the training data, and then metagene values are computed using the principal components of the training data and additional expression data. Bayesian fitting of binary probit regression models to the training data then permits an assessment of the relevance of the metagene signatures in within-sample classification, and estimation and uncertainty assessments for the binary regression weights mapping metagenes to probabilities of relative pathway status. Predictions of sensitivity to an anti-cancer agent are then evaluated, producing estimated relative probabilities—and associated measures of uncertainty—of sensitivity to an anti-cancer agent across the validation samples. Hierarchical clustering of sensitivity to anti-cancer agent predictions may be performed using Gene Cluster 3.0 testing the null hypothesis, which is that the survival curves are identical in the overall population.

In one embodiment, the each statistical tree model generated by the methods described herein comprises 2, 3, 4, 5, 6 or more nodes. In one embodiment of the methods described herein for defining a statistical tree model predictive of sensitivity/resistance to a therapeutic, the resulting model predicts cancer sensitivity to an anti-cancer agent with at least 70%, 80%, 85%, or 90% or higher accuracy. In another embodiment, the model predicts sensitivity to an anti-cancer agent with greater accuracy than clinical variables. In one embodiment, the clinical variables are selected from age of the subject, gender of the subject, tumor size of the sample, stage of cancer disease, histological subtype of the sample and smoking history of the subject. In one embodiment, the cluster of genes that define each metagene comprise at least 3, 4, 5, 6, 7, 8, 9, 10, 12 or 15 genes. In one embodiment, the correlation-based clustering is Markov chain correlation-based clustering or K-means clustering.

### Diagnostic Business Methods

One aspect of the invention provides methods of conducting a diagnostic business, including a business that provides a health care practitioner with diagnostic information for the treatment of a subject afflicted with cancer. One such method comprises one, more than one, or all of the following steps: (i) obtaining an tumor sample from the subject; (ii) determining the expression level of multiple genes in the sample; (iii) defining the value of one or more metagenes from the expression levels of step (ii), wherein each metagene is defined by extracting a single dominant value using single value decomposition (SVD) from a cluster of genes associated with sensitivity to an anti-cancer agent; (iv) averaging the predictions of one or more statistical tree models applied to the values, wherein each model includes one or more nodes, each node representing a metagene, each node including a statistical predictive probability of sensitivity to an anti-cancer agent, wherein at least one metagene is one of metagenes 1-7; and (v) providing the health care practitioner with the prediction from step (iv).

In one embodiment, obtaining a tumor sample from the subject is effected by having an agent of the business (or a subsidiary of the business) remove a tumor sample from the subject, such as by a surgical procedure. In another embodiment, obtaining a tumor sample from the subject comprises receiving a sample from a health care practitioner, such as by shipping the sample, preferably frozen. In one embodiment, the sample is a cellular sample, such as a mass of tissue. In one embodiment, the sample comprises a nucleic acid sample, such as a DNA, cDNA, mRNA sample, or combinations thereof, which was derived from a cellular tumor sample from the subject. In one embodiment, the prediction from step (iv) is provided to a health care practitioner, to the patient, or to any other business entity that has contracted with the subject.

In one embodiment, the method comprises billing the subject, the subject's insurance carrier, the health care practitioner, or an employer of the health care practitioner. A government agency, whether local, state or federal, may also be billed for the services. Multiple parties may also be billed for the service.

In some embodiments, all the steps in the method are carried out in the same general location. In certain embodiments, one or more steps of the methods for conducting a diagnostic business are performed in different locations. In one embodiment, step (ii) is performed in a first location, and step (iv) is performed in a second location, wherein the first location is remote to the second location. The other steps may be performed at either the first or second location, or in other locations. In one embodiment, the first location is remote to the second location. A remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. In one embodiment, two locations that are remote relative to each other are at least 1, 2, 3, 4, 5, 10, 20, 50, 100, 200, 500, 1000, 2000 or 5000 km apart. In another embodiment, the two locations are in different countries, where one of the two countries is the United States.

Some specific embodiments of the methods described herein where steps are performed in two or more locations comprise one or more steps of communicating information between the two locations. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In one specific embodiment, the method comprises one or more data transmission steps between the locations. In one embodiment, the data transmission step occurs via an electronic communication link, such as the internet. In one embodiment, the data transmission step from the first to the second location comprises experimental parameter data, such as the level of gene expression of multiple genes. In some embodiments, the data transmission step from the second location to the first location comprises data transmission to intermediate locations. In one specific embodiment, the method comprises one or more data transmission substeps from the second location to one or more intermediate locations and one or more data transmission substeps from one or more intermediate locations to the first location, wherein the intermediate locations are remote to both the first and second locations. In another embodiment, the method comprises a data transmission step in which a result from gene expression is transmitted from the second location to the first location.

In one embodiment, the methods of conducting a diagnostic business comprise the step of determining if the subject carries an allelic form of a gene whose presence correlates to sensitivity or resistance to a chemotherapeutic agent. This may be achieved by analyzing a nucleic acid sample from the patient and determining the DNA sequence of the allele. Any technique known in the art for determining the presence of mutations or polymorphisms may be used. The method is not limited to any particular mutation or to any particular allele or gene. For example, mutations in the epidermal growth factor receptor (EGFR) gene are found in human lung adenocarcinomas and are associated with sensitivity to the tyrosine kinase inhibitors gefitinib and erlotinib. (See, e.g., Yi et al. Proc Natl Acad Sci USA. 2006 May 16; 103(20):7817-22; Shimato et al. Neuro-oncol. 2006 April; 8(2):137-44). Similarly, mutations in breast cancer resistance protein (BCRP) modulate the resistance of cancer cells to BCRP-substrate anticancer agents (Yanase et al., Cancer Lett. 2006 Mar. 8; 234(1):73-80).

### Arrays and Gene Chips and Kits Comprising Thereof

Arrays and microarrays which contain the gene expression profiles for determining responsivity to platinum-based therapy and/or responsivity to salvage agents are also encompassed within the scope of this invention. Methods of making arrays are well-known in the art and as such, do not need to be described in detail here.

Such arrays can contain the profiles of at least 5, 10, 15, 25, 50, 75, 100, 150, or 200 genes as disclosed in Table 1. Accordingly, arrays for detection of responsivity to particular therapeutic agents can be customized for diagnosis or treatment of ovarian cancer. The array can be packaged as part of kit comprising the customized array itself and a set of instructions for how to use the array to determine an individual's responsivity to a specific cancer therapeutic agent.

Also provided are reagents and kits thereof for practicing one or more of the above described methods. The subject reagents and kits thereof may vary greatly. Reagents of interest include reagents specifically designed for use in production of the above described metagene values.

One type of such reagent is an array probe of nucleic acids, such as a DNA chip, in which the genes defining the metagenes in the therapeutic efficacy predictive tree models are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

The DNA chip is convenient to compare the expression levels of a number of genes at the same time. DNA chip-based expression profiling can be carried out, for example, by the method as disclosed in “Microarray Biochip Technology” (Mark Schena, Eaton Publishing, 2000). A DNA chip comprises immobilized high-density probes to detect a number of genes. Thus, the expression levels of many genes can be estimated at the same time by a single-round analysis. Namely, the expression profile of a specimen can be determined with a DNA chip. A DNA chip may comprise probes, which have been spotted thereon, to detect the expression level of the metagene-defining genes of the present invention. A probe may be designed for each marker gene selected, and spotted on a DNA chip. Such a probe may be, for example, an oligonucleotide comprising 5-50 nucleotide residues. A method for synthesizing such oligonucleotides on a DNA chip is known to those skilled in the art. Longer DNAs can be synthesized by PCR or chemically. A method for spotting long DNA, which is synthesized by PCR or the like, onto a glass slide is also known to those skilled in the art. A DNA chip that is obtained by the method as described above can be used estimating the efficacy of a therapeutic agent in treating a subject afflicted with cancer according to the present invention.

DNA microarray and methods of analyzing data from microarrays are well-described in the art, including in DNA Microarrays: A Molecular Cloning Manual, Ed. by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); DNA Microarrays: A Practical Approach, Vol. 205 by Schema (Oxford University Press, 1999); and Methods of Microarray Data Analysis II, ed. by Lin et al. (Kluwer Academic Publishers, 2002).

One aspect of the invention provides a gene chip having a plurality of different oligonucleotides attached to a first surface of the solid support and having specificity for a plurality of genes, wherein at least 50% of the genes are common to those of metagenes 1, 2, 3, 4, 5, 6 and/or 7. In one embodiment, at least 70%, 80%, 90% or 95% of the genes in the gene chip are common to those of metagenes 1, 2, 3, 4, 5, 6 and/or 7.

One aspect of the invention provides a kit comprising: (a) any of the gene chips described herein; and (b) one of the computer-readable mediums described herein.

In some embodiments, the arrays include probes for at least 2, 3, 4, 5, 6, 7, 8, 9, 10, 15, 20, 25, 30, 40, or 50 of the genes listed in Table 1. In certain embodiments, the number of genes that are from Table 1 that are represented on the array is at least 5, at least 10, at least 25, at least 50, at least 75 or more, including all of the genes listed in the table. Where the subject arrays include probes for additional genes not listed in the tables, in certain embodiments the number % of additional genes that are represented does not exceed about 50%, 40%, 30%, 20%, 15%, 10%, 8%, 6%, 5%, 4%, 3%, 2% or 1%. In some embodiments, a great majority of genes in the collection are genes that define the metagenes of the invention, where by great majority is meant at least about 75%, usually at least about 80% and sometimes at least about 85, 90, 95% or higher, including embodiments where 100% of the genes in the collection are metagene-defining genes.

The kits of the subject invention may include the above described arrays. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

### Computer Readable Media Comprising Gene Expression Profiles

The invention also contemplates computer readable media that comprises gene expression profiles. Such media can contain all of part of the gene expression profiles of the genes listed in Table 1. The media can be a list of the genes or contain the raw data for running a user's own statistical calculation, such as the methods disclosed herein.

### Program Products/Systems

Another aspect of the invention provides a program product (i.e., software product) for use in a computer device that executes program instructions recorded in a computer-readable medium to perform one or more steps of the methods described herein, such for estimating the efficacy of a therapeutic agent in treating a subject afflicted with cancer.

One aspect of the invention provides a computer readable medium having computer readable program codes embodied therein, the computer readable medium program codes performing one or more of the following functions: defining the value of one or more metagenes from the expression levels genes; defining a metagene value by extracting a single dominant value using singular value decomposition (SVD) from a cluster of genes associated tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

Another related aspect of the invention provides kits comprising the program product or the computer readable medium, optionally with a computer system. One aspect of the invention provides a system, the system comprising: a computer; a computer readable medium, operatively coupled to the computer, the computer readable medium program codes performing one or more of the following functions: defining the value of one or more metagenes from the expression levels genes; defining a metagene value by extracting a single dominant value using singular value decomposition (SVD) from a cluster of genes associated tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

In one embodiment, the program product comprises: a recordable medium; and a plurality of computer-readable instructions executable by the computer device to analyze data from the array hybridization steps, to transmit array hybridization from one location to another, or to evaluate genome-wide location data between two or more genomes. Computer readable media include, but are not limited to, CD-ROM disks (CD-R, CD-RW), DVD-RAM disks, DVD-RW disks, floppy disks and magnetic tape.

A related aspect of the invention provides kits comprising the program products described herein. The kits may also optionally contain paper and/or computer-readable format instructions and/or information, such as, but not limited to, information on DNA microarrays, on tutorials, on experimental procedures, on reagents, on related products, on available experimental data, on using kits, on chemotherapeutic agents including there toxicity, and on other information. The kits optionally also contain in paper and/or computer-readable format information on minimum hardware requirements and instructions for running and/or installing the software. The kits optionally also include, in a paper and/or computer readable format, information on the manufacturers, warranty information, availability of additional software, technical services information, and purchasing information. The kits optionally include a video or other viewable medium or a link to a viewable format on the internet or a network that depicts the use of the use of the software, and/or use of the kits. The kits also include packaging material such as, but not limited to, styrofoam, foam, plastic, cellophane, shrink wrap, bubble wrap, paper, cardboard, starch peanuts, twist ties, metal clips, metal cans, drierite, glass, and rubber.

The analysis of data, as well as the transmission of data steps, can be implemented by the use of one or more computer systems. Computer systems are readily available. The processing that provides the displaying and analysis of image data for example, can be performed on multiple computers or can be performed by a single, integrated computer or any variation thereof. For example, each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically includes volatile semiconductor random access memory (RAM).

Each computer typically includes a program product reader that accepts a program product storage device from which the program product reader can read data (and to which it can optionally write data). The program product reader can include, for example, a disk drive, and the program product storage device can include a removable storage medium such as, for example, a magnetic floppy disk, an optical CD-ROM disc, a CD-R disc, a CD-RW disc and a DVD data disc. If desired, computers can be connected so they can communicate with each other, and with other connected computers, over a network. Each computer can communicate with the other connected computers over the network through a network interface that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network. In the network method, the computer receives data including program steps into the memory through the network interface after network communication has been established over the network connection by well known methods understood by those skilled in the art. The computer that implements the client side processing, and the computer that implements the server side processing or any other computer device of the system, can include any conventional computer suitable for implementing the functionality described herein.

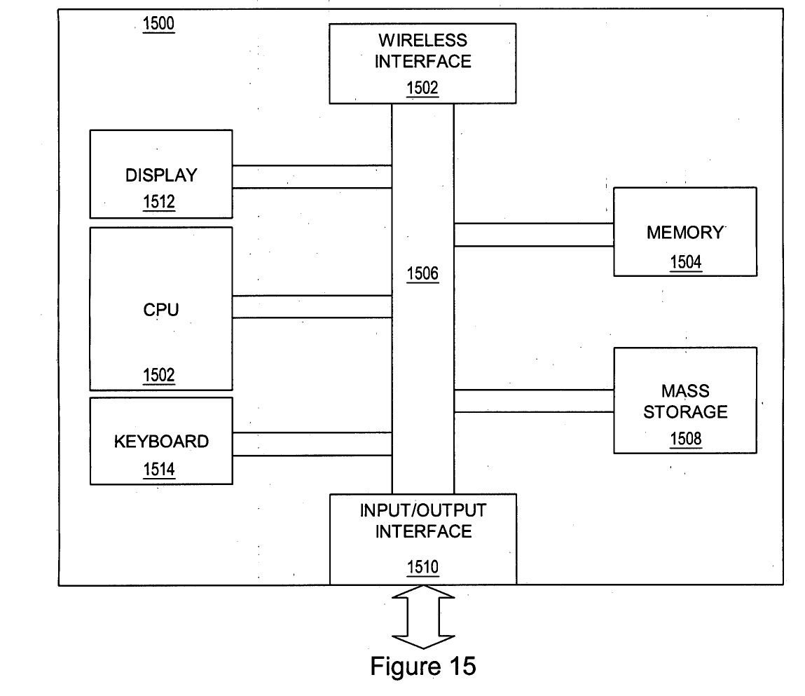

FIG. 15 shows a functional block diagram of general purpose computer system 1500 for performing the functions of the software according to an illustrative embodiment of the invention. The exemplary computer system 1500 includes a central processing unit (CPU) 3002, a memory 1504, and an interconnect bus 1506. The CPU 1502 may include a single microprocessor or a plurality of microprocessors for configuring computer system 1500 as a multi-processor system. The memory 1504 illustratively includes a main memory and a read only memory. The computer 1500 also includes the mass storage device 1508 having, for example, various disk drives, tape drives, etc. The main memory 1504 also includes dynamic random access memory (DRAM) and high-speed cache memory. In operation, the main memory 1504 stores at least portions of instructions and data for execution by the CPU 1502.

The mass storage 1508 may include one or more magnetic disk or tape drives or optical disk drives, for storing data and instructions for use by the CPU 1502. At least one component of the mass storage system 1508, preferably in the form of a disk drive or tape drive, stores one or more databases, such as databases containing of transcriptional start sites, genomic sequence, promoter regions, or other information.

The mass storage system 1508 may also include one or more drives for various portable media, such as a floppy disk, a compact disc read only memory (CD-ROM), or an integrated circuit non-volatile memory adapter (i.e., PC-MCIA adapter) to input and output data and code to and from the computer system 1500.

The computer system 1500 may also include one or more input/output interfaces for communications, shown by way of example, as interface 1510 for data communications via a network. The data interface 1510 may be a modem, an Ethernet card or any other suitable data communications device. To provide the functions of a computer system according to FIG. 15 the data interface 1510 may provide a relatively high-speed link to a network, such as an intranet, internet, or the Internet, either directly or through an another external interface. The communication link to the network may be, for example, optical, wired, or wireless (e.g., via satellite or cellular network). Alternatively, the computer system 1500 may include a mainframe or other type of host computer system capable of Web-based communications via the network.

The computer system 1500 also includes suitable input/output ports or use the interconnect bus 1506 for interconnection with a local display 1512 and keyboard 1514 or the like serving as a local user interface for programming and/or data retrieval purposes. Alternatively, server operations personnel may interact with the system 1500 for controlling and/or programming the system from remote terminal devices via the network.

The computer system 1500 may run a variety of application programs and stores associated data in a database of mass storage system 1508. One or more such applications may enable the receipt and delivery of messages to enable operation as a server, for implementing server functions relating to obtaining a set of nucleotide array probes tiling the promoter region of a gene or set of genes.

The components contained in the computer system 1500 are those typically found in general purpose computer systems used as servers, workstations, personal computers, network terminals, and the like. In fact, these components are intended to represent a broad category of such computer components that are well known in the art.

It will be apparent to those of ordinary skill in the art that methods involved in the present invention may be embodied in a computer program product that includes a computer usable and/or readable medium. For example, such a computer usable medium may consist of a read only memory device, such as a CD ROM disk or conventional ROM devices, or a random access memory, such as a hard drive device or a computer diskette, having a computer readable program code stored thereon.

The following examples are provided to illustrate aspects of the invention but are not intended to limit the invention in any manner.

### EXAMPLES

### Example 1A Gene Expression Based Predictor of Sensitivity to Docetaxel

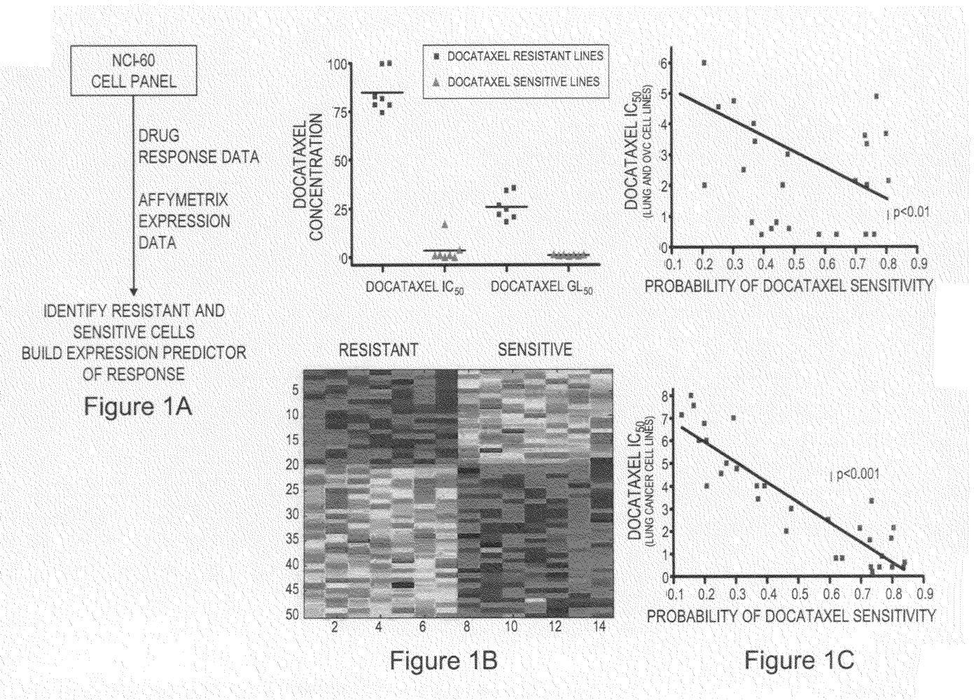

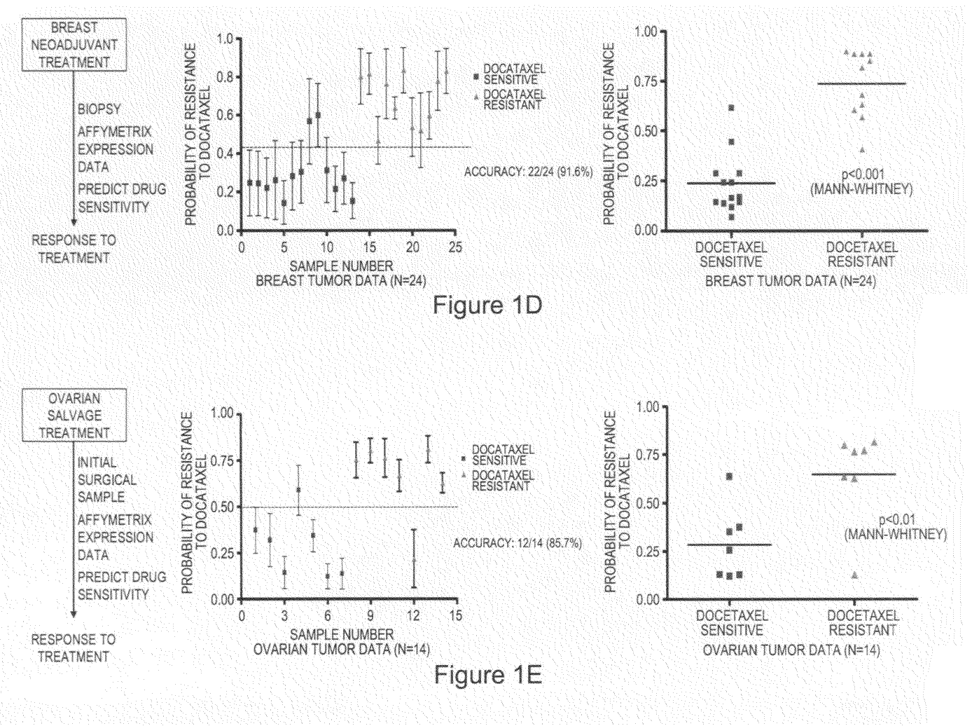

To develop predictors of cytotoxic chemotherapeutic drug response, we used an approach similar to previous work analyzing the NCI-60 panel,⁴⁹ first identifying cell lines that were most resistant or sensitive to docetaxel (FIG. 1A, B) and then genes whose expression most highly correlated with drug sensitivity, using Bayesian binary regression analysis to develop a model that differentiates a pattern of docetaxel sensitivity from resistance. A gene expression signature consisting of 50 genes was identified that classified on the basis of docetaxel sensitivity (FIG. 1B, bottom panel).

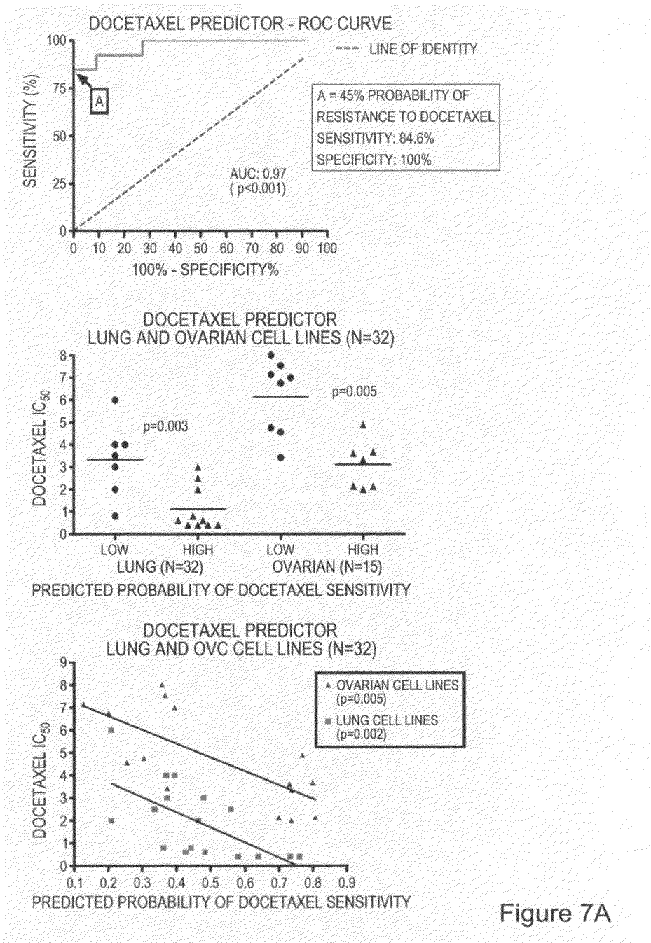

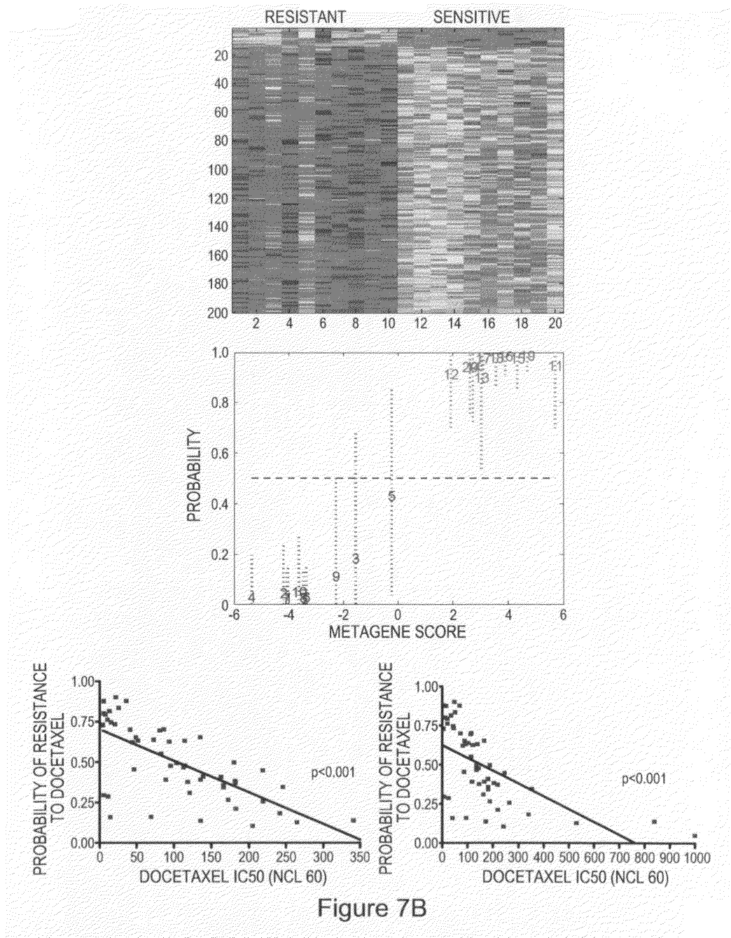

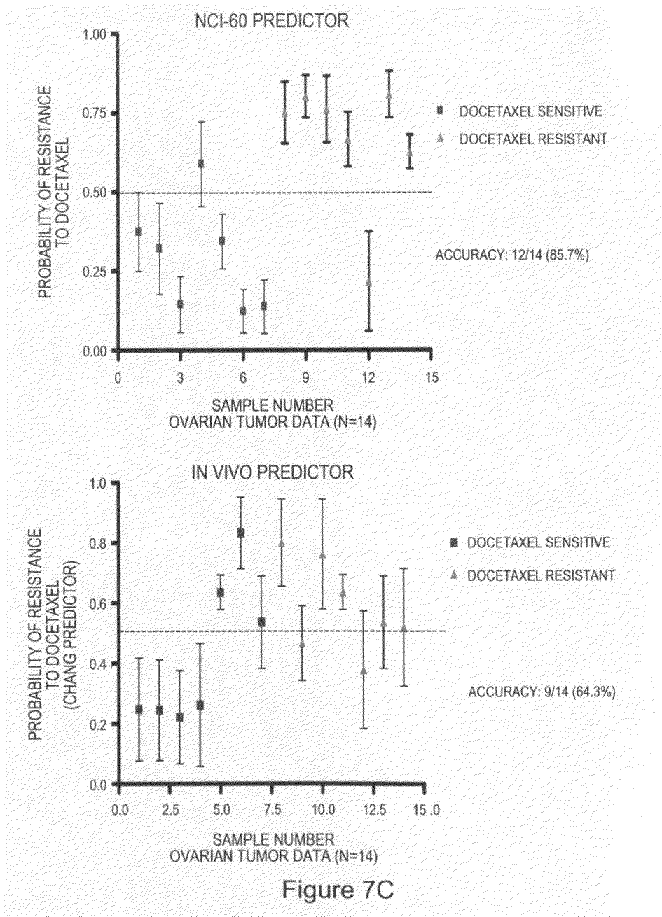

In addition to leave-one-out cross validation, we utilized an independent dataset derived from docetaxel sensitivity assays in a series of 30 lung and ovarian cancer cell lines for further validation. As shown in FIG. 1C (top panel), the correlation between the predicted probability of sensitivity to docetaxel (in both lung and ovarian cell lines) and the respective IC50 for docetaxel confirmed the capacity of the docetaxel predictor to predict sensitivity to the drug in cancer cell lines (FIG. 7). In each case, the accuracy exceeded 80%. Finally, we made use of a second independent dataset that measured docetaxel sensitivity in a series of 29 lung cancer cell lines (Gemma A, GEO accession number: GSE 4127). As shown in FIG. 1C (bottom panel), the docetaxel sensitivity model developed from the NCI-60 panel again predicted sensitivity in this independent dataset, again with an accuracy exceeding 80%.

### Example 2 Utilization of the Expression Signature to Predict Docetaxel Response in Patients

The development of a gene expression signature capable of predicting in vitro docetaxel sensitivity provides a tool that might be useful in predicting response to the drug in patients. We have made use of published studies with clinical and genomic data that linked gene expression data with clinical response to docetaxel in a breast cancer neoadjuvant study⁵⁰ (FIG. 1D) to test the capacity of the in vitro docetaxel sensitivity predictor to accurately identify those patients that responded to docetaxel. Using a 0.45 predicted probability of response as the cut-off for predicting positive response, as determined by ROC curve analysis (FIG. 7A), the in vitro generated profile correctly predicted docetaxel response in 22 out of 24 patient samples, achieving an overall accuracy of 91.6% (FIG. 1D). Applying a Mann-Whitney U test for statistical significance demonstrates the capacity of the predictor to distinguish resistant from sensitive patients (FIG. 1D, right panel). We extended this further by predicting the response to docetaxel as salvage therapy for ovarian cancer. As shown in FIG. 1E, the prediction of response to docetaxel in patients with advanced ovarian cancer achieved an accuracy exceeding 85% (FIG. 1E, middle panel). Further, an analysis of statistical significance demonstrated the capacity of the predictors to distinguish patients with resistant versus sensitive disease (FIG. 1E, right panel).

We also performed a complementary analysis using the patient response data to generate a predictor and found that the in vivo generated signature of response predicted sensitivity of NCI-60 cell lines to docetaxel (FIG. 7B). This crossover is further emphasized by the fact that the genes represented in either the initial in vitro generated docetaxel predictor or the alternative in vivo predictor exhibit considerable overlap. Importantly, both predictors link to expected targets for docetaxel including bcl-2, TRAG, erb-B2, and tubulin genes, all previously described to be involved in taxane chemoresistance⁵¹⁻⁵⁴ (Table 1). We also note that the predictor of docetaxel sensitivity developed from the NCI-60 data was more accurate in predicting patient response in the ovarian samples than the predictor developed from the breast neoadjuvant patient data (85.7% vs. 64.3%) (FIG. 7C).

### Example 3 Development of a Panel of Gene Expression Signatures that Predict Sensitivity to Chemotherapeutic Drugs

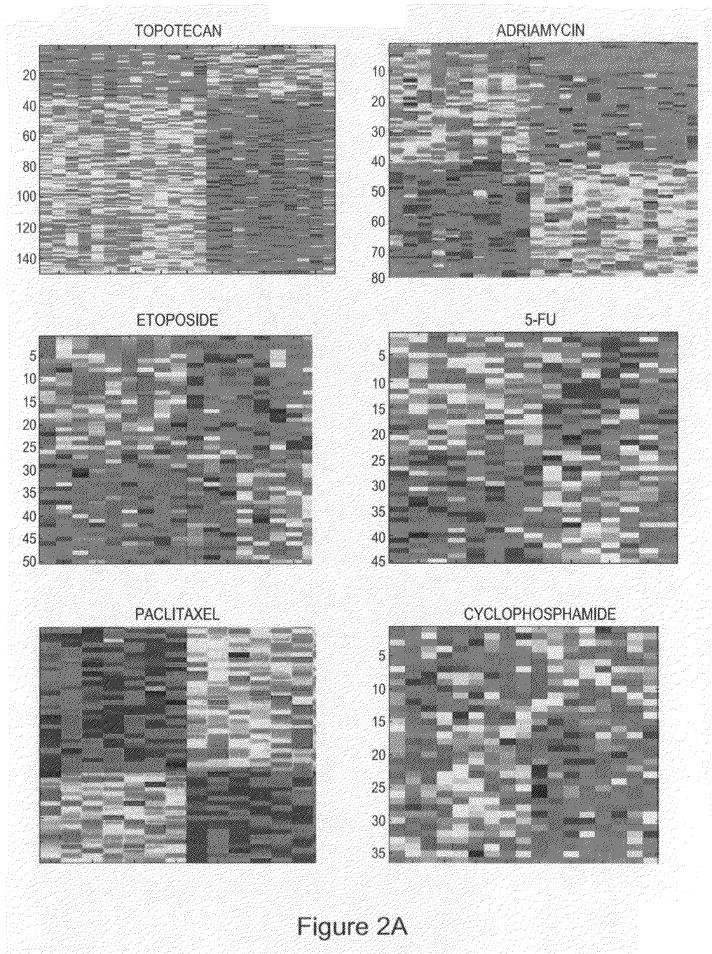

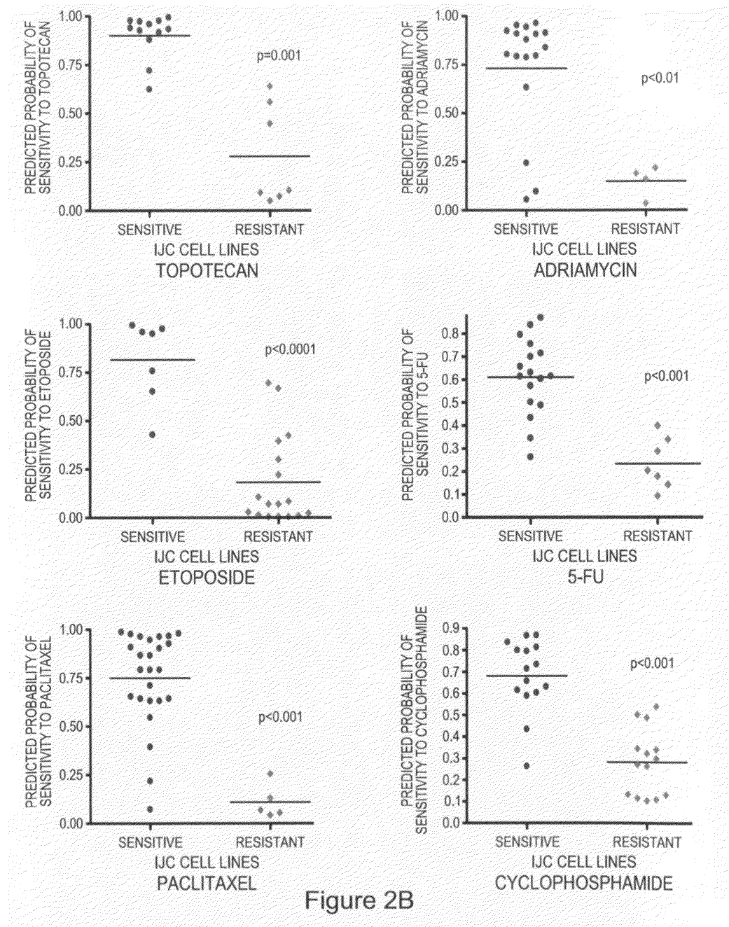

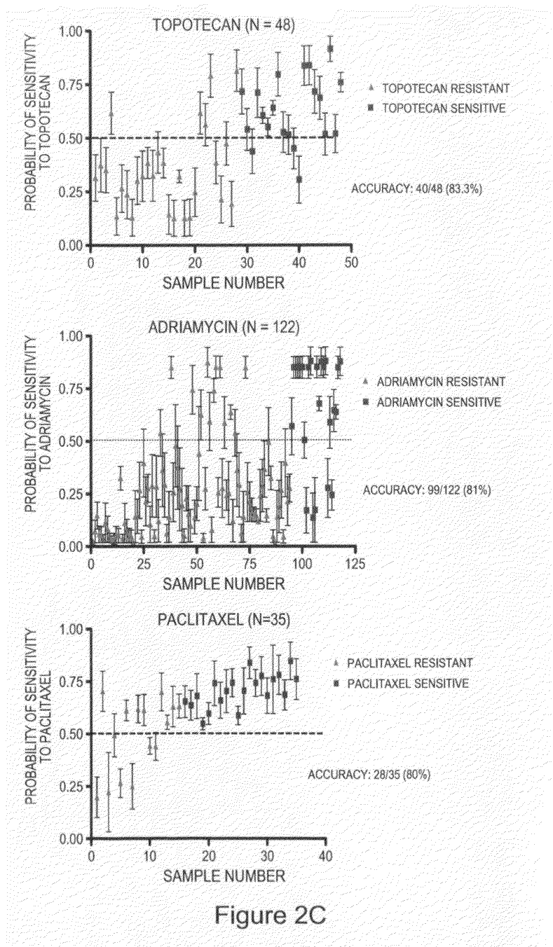

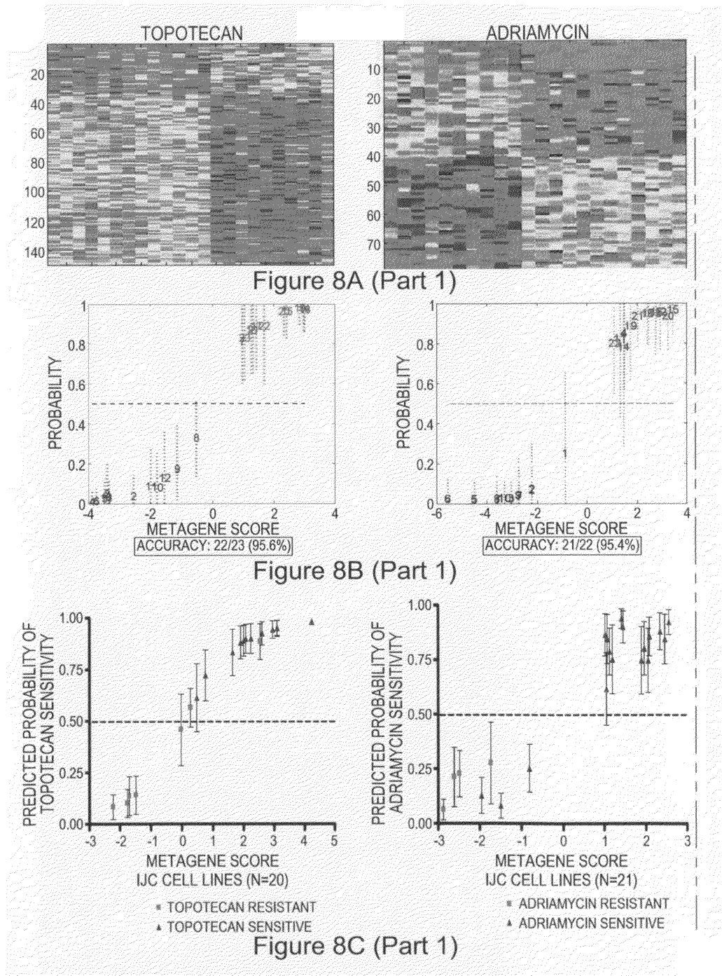

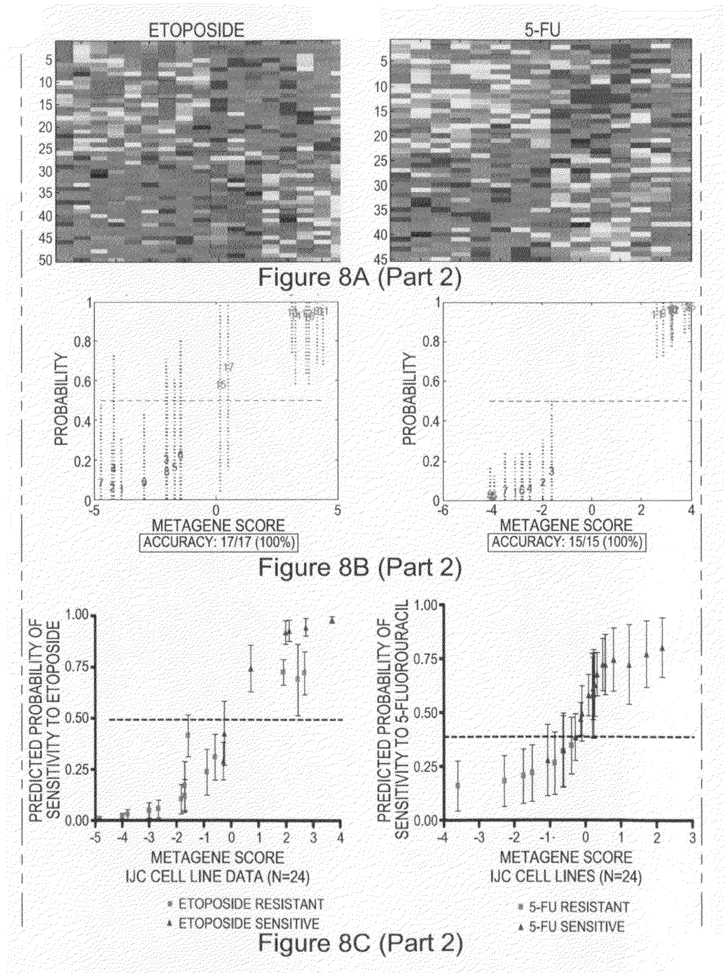

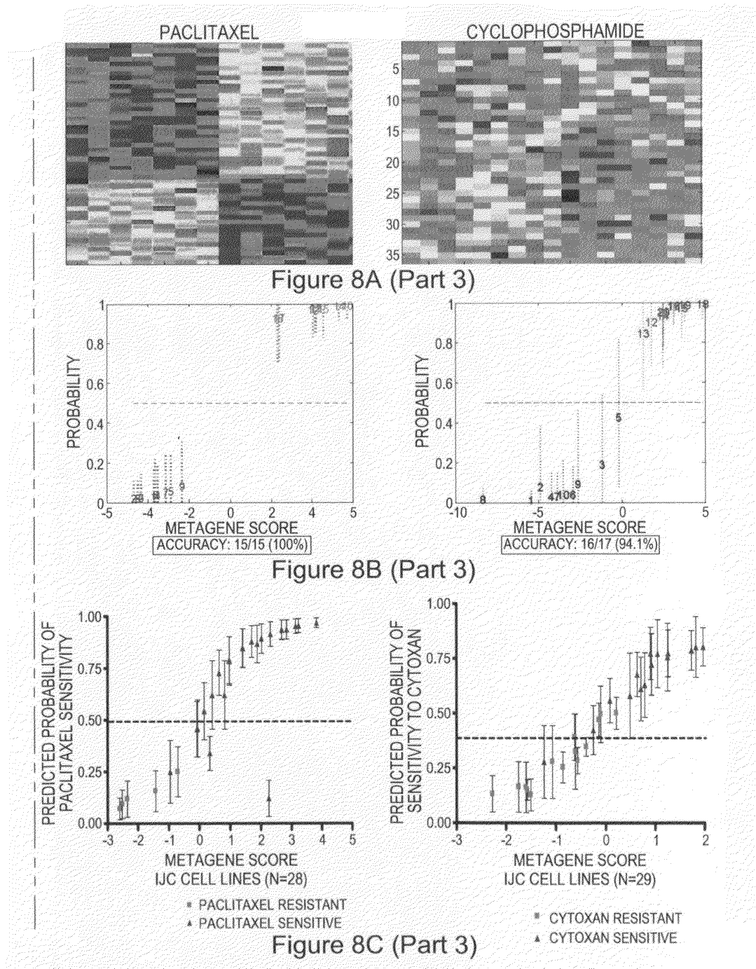

Given the development of a docetaxel response predictor, we have examined the NCI-60 dataset for other opportunities to develop predictors of chemotherapy response. Shown in FIG. 2A are a series of expression profiles developed from the NCI-60 dataset that predict response to topotecan, adriamycin, etoposide, 5-fluorouracil (5-FU), taxol, and cyclophosphamide. In each case, the leave-one-out cross validation analyses demonstrate a capacity of these profiles to accurately predict the samples utilized in the development of the predictor (FIG. 8, middle panel). Each profile was then further validated using in vitro response data from independent datasets; in each case, the profile developed from the NCI-60 data was capable of accurately (>85%) predicting response in the separate dataset of approximately 30 cancer cell lines for which the dose response information and relevant Affymetrix U133A gene expression data is publicly available³⁷ (FIG. 8 (bottom panel) and Table 2). Once again, applying a Mann-Whitney U test for statistical significance demonstrates the capacity of the predictor to distinguish resistant from sensitive patients (FIG. 2B).

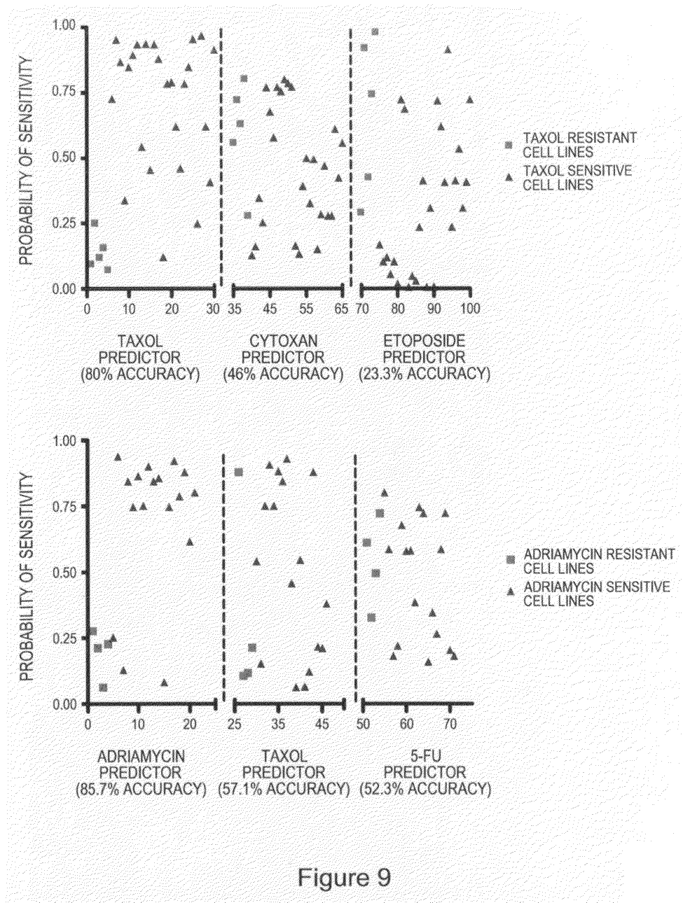

In addition to the capacity of each signature to distinguish cells that are sensitive or resistant to a particular drug, we also evaluated the extent to which a signature was also specific for an individual chemotherapeutic agent. From the example shown in FIG. 9, using the validations of chemosensitivity seen in the independent European (IJC) cell line data it is clear that each of the signatures is specific for the drug that was used to develop the predictor. In each case, individual predictors of response to the various cytotoxic drugs was plotted against cell lines known to be sensitive or resistant to a given chemotherapeutic agent (e.g., adriamycin, paclitaxel).

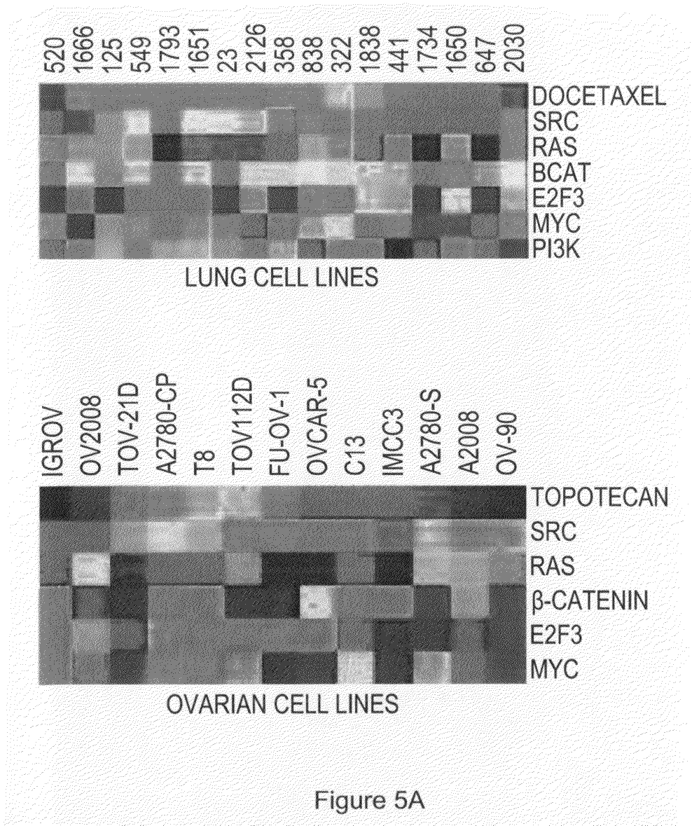

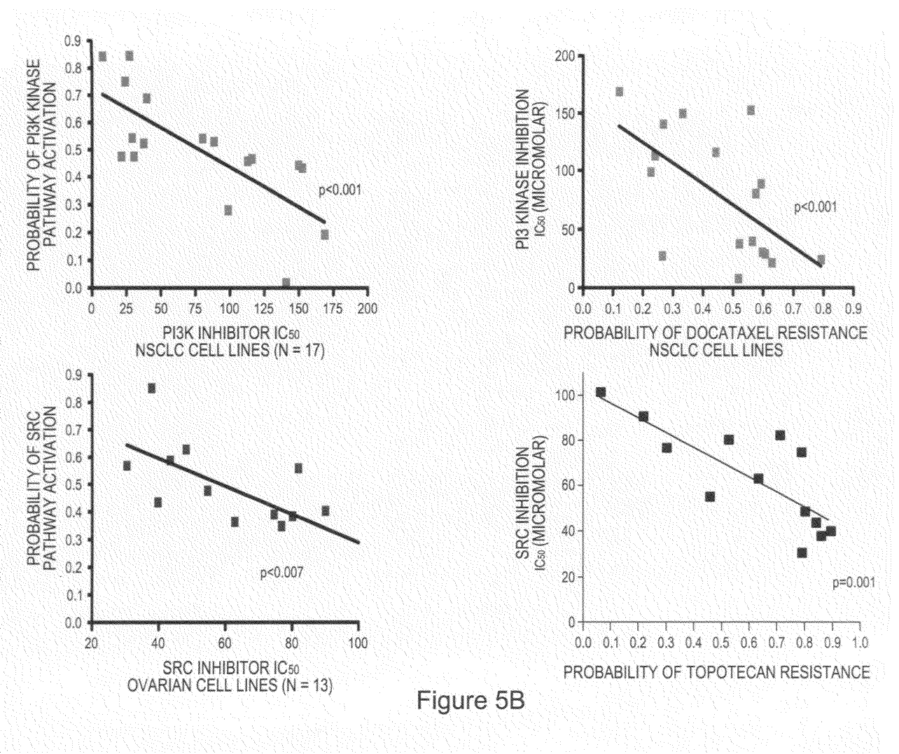

Given the ability of the in vitro developed gene expression profiles to predict response to docetaxel in the clinical samples, we extended this approach to test the ability of additional signatures to predict response to commonly used salvage therapies for ovarian cancer and an independent dataset of samples from adriamycin treated patients (Evans W, GSE650, GSE651). As shown in FIG. 5C, each of these predictors was capable of accurately predicting the response to the drugs in patient samples, achieving an accuracy in excess of 81% overall. In each case, the positive and negative predictive values confirm the validity and clinical utility of the approach (Table 2).

### Example 4 Chemotherapy Response Signatures Predict Response to Multi-Drug Regimens

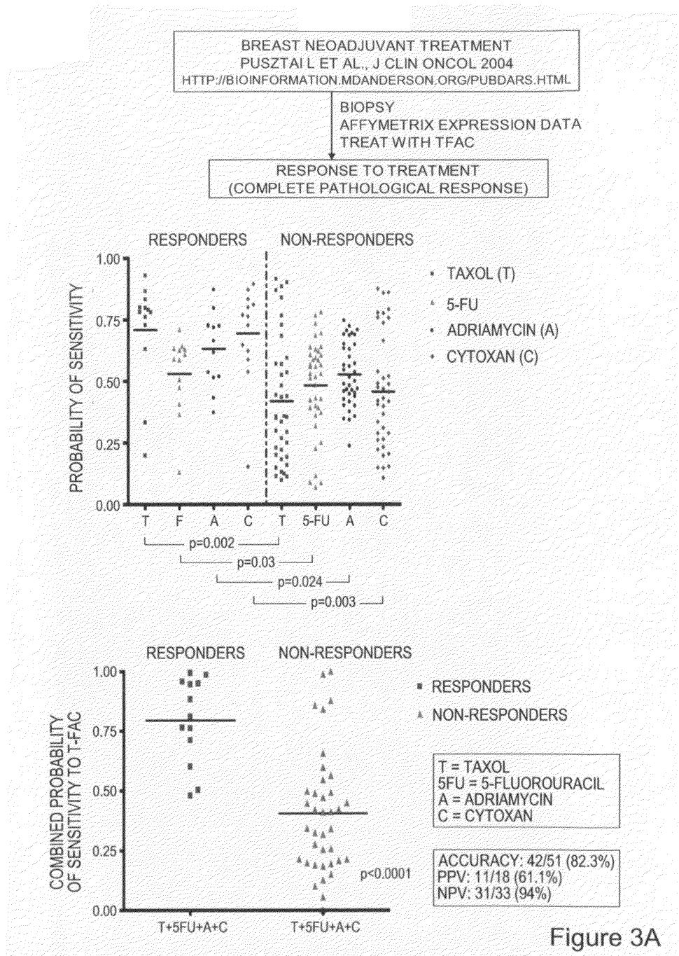

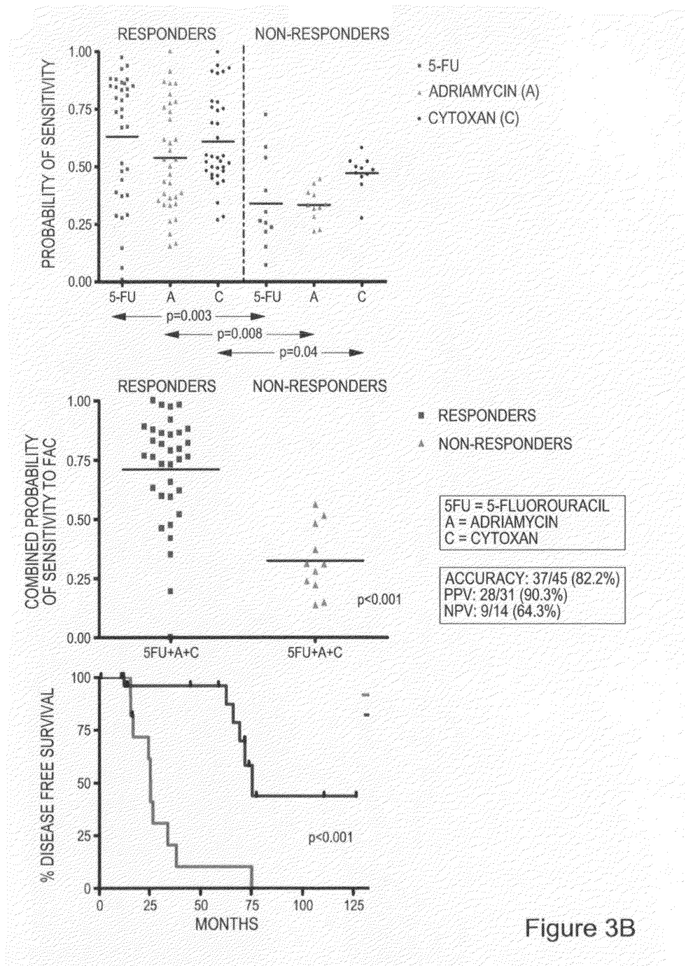

Many therapeutic regimens make use of combinations of chemotherapeutic drugs raising the question as to the extent to which the signatures of individual therapeutic response will also predict response to a combination of agents. To address this question, we have made use of data from a breast neoadjuvant treatment that involved the use of paclitaxel, 5-fluorouracil, adriamycin, and cyclophosphamide (TFAC)^{55,56} (FIG. 3A). Using available data from the 51 patients to then predict response with each of the single agent signatures (paclitaxel, 5-FU, adriamycin and cyclophosphamide) developed from the NCI-60 cell line analysis; we then compared to the clinical outcome information which was represented as complete pathologic response. As shown in FIG. 3A (middle panel), the predicted response based on each of the individual chemosensitivity signatures indicated a significant distinction between the responders (n=13) and non-responders (n=38) with the exception of 5-fluorouracil. Importantly, the combined probability of sensitivity to the four agents in this TFAC neoadjuvant regimen was calculated using the probability theorem and it is clear from this analysis that the prediction of response based on a combined probability of sensitivity, built from the individual chemosensitivity predictions yielded a statistically significant (p<0.0001, Mann Whitney U) distinction between the responders and non-responders (FIG. 3A, right panel).

As a further validation of the capacity to predict response to combination therapy, we have made use of gene expression data generated from a collection of breast cancer (n=45) samples from patients who received 5-fluorouracil, adriamycin and cyclophosphamide (FAC) in the adjuvant chemotherapy set. As shown in FIG. 3B (left panel), the predicted response based on signatures for 5-FU, adriamycin, and cyclophosphamide indicated a significant distinction between the responders (n=34) and non-responders (n=11) for each of the single agent predictors. Furthermore, the combined probability of sensitivity to the three agents in the FAC regimen was calculated and shown in the middle panel of FIG. 3B. It is evident from this analysis that the prediction of response based on a combined probability of sensitivity to the FAC regimen yielded a clear, significant (p<0.001, Mann Whitney U) distinction between the responders and non-responders (accuracy: 82.2%, positive predictive value: 90.3%, negative predictive value: 64.3%). We note that while it is difficult to interpret the prediction of clinical response in the adjuvant setting since many of these patients were likely free of disease following surgery, the accurate identification of non-responders is a clear endpoint that does confirm the capacity of the signatures to predict clinical response.

As a further measure of the relevance of the predictions, we examined the prognostic significance of the ability to predict response to FAC. As shown in FIG. 3B (right panel), there was a clear distinction in the population of patients identified as sensitive or resistant to FAC, as measured by disease-free survival. These results, taken together with the accuracy of prediction of response in the neoadjuvant setting where clinical endpoints are uncomplicated by confounding variables such as prior surgery, and results of the single agent validations, leads us to conclude that the signatures of chemosensitivity generated from the NCI-60 panel do indeed have the capacity to predict therapeutic response in patients receiving either single agent or combination chemotherapy (Table 3).

When comparing individual genes that constitute the predictors, it was interesting to observe that the gene coding for MAP-Tau, described previously as a determinant of paclitaxel sensitivity,⁵⁶ was also identified as a discriminator gene in the paclitaxel predictor generated using the NCI-60 data. Although, similar to the docetaxel example described earlier, a predictor for TFAC chemotherapy developed using the NCI-60 data was superior to the ability of the MAP-Tau based predictor described by Pusztai et al (Table 4). Similarly, p53, methyltetrahydrofolate reductase gene and DNA repair genes constitute the 5-fluorouracil predictor, and excision repair mechanism genes (e.g., ERCC4), retinoblastoma pathway genes, and bcl-2 constitute the adriamycin predictor, consistent with previous reports (Table 1).

### Example 5 Patterns of Predicted Chemotherapy Response Across a Spectrum of Tumors

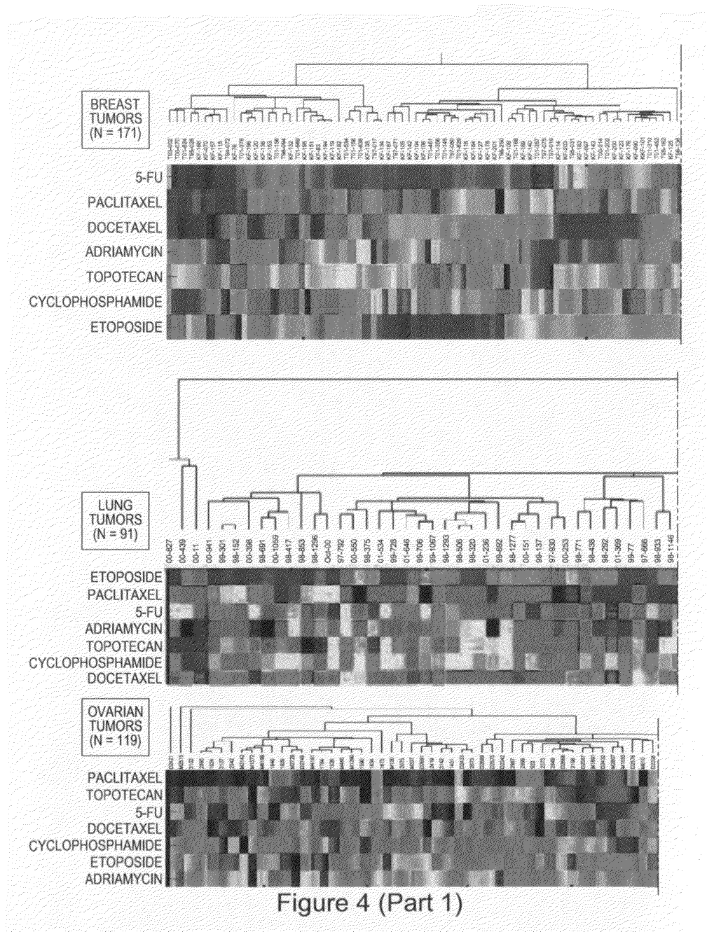

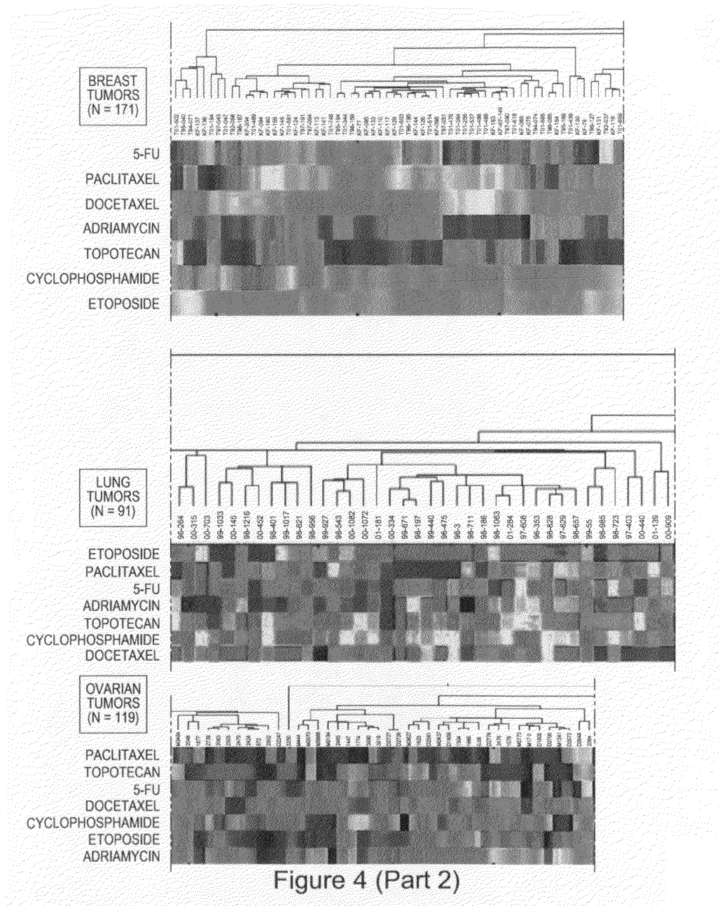

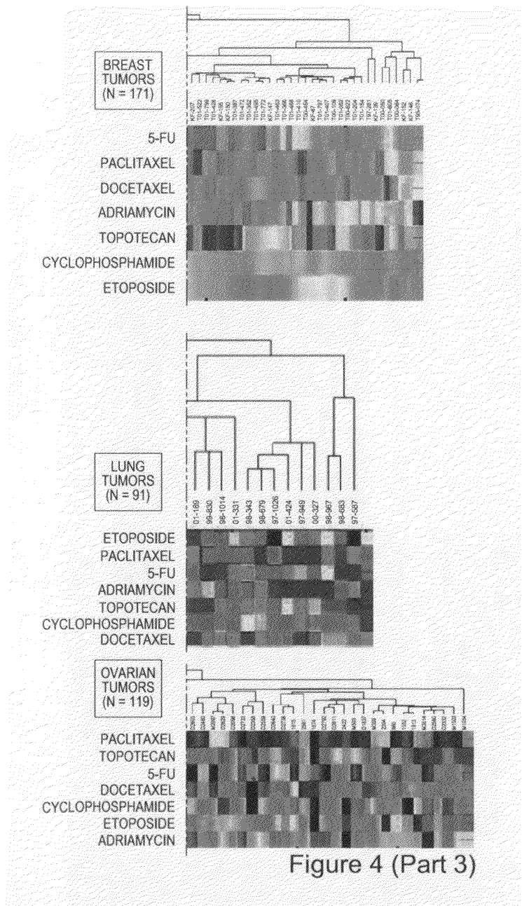

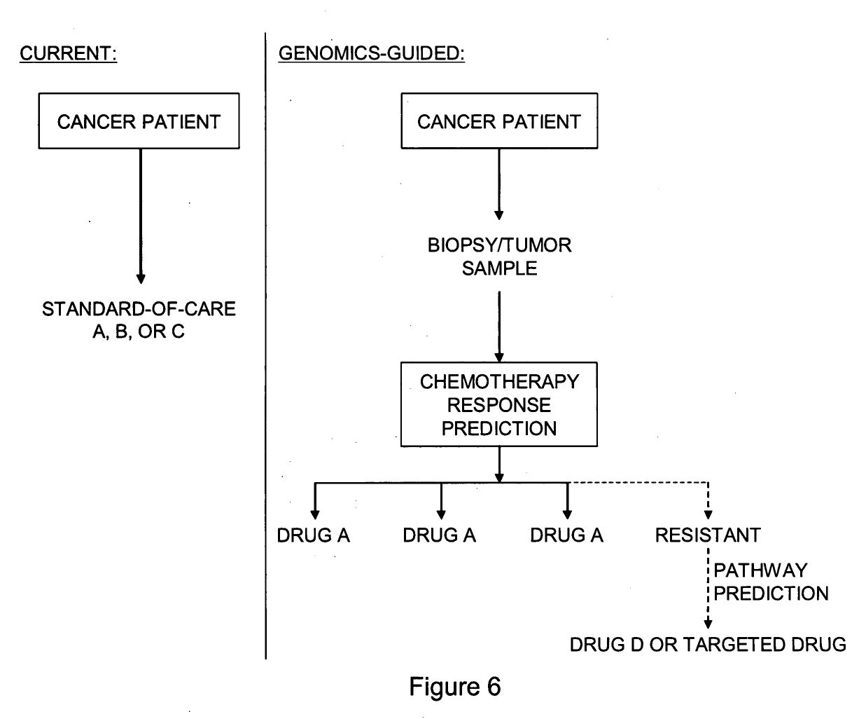

The availability of genomic-based predictors of chemotherapy response could potentially provide an opportunity for a rational approach to selection of drugs and combination of drugs. With this in mind, we have utilized the panel of chemotherapy response predictors described in FIG. 6 to profile the potential options for use of these agents, by predicting the likelihood of sensitivity to the seven agents in a large collection of breast, lung, and ovarian tumor samples. We then clustered the samples according to patterns of predicted sensitivity to the various chemotherapeutics, and plotted a heatmap in which high probability of sensitivity response is indicated by red and low probability or resistance is indicated by blue (FIG. 4).

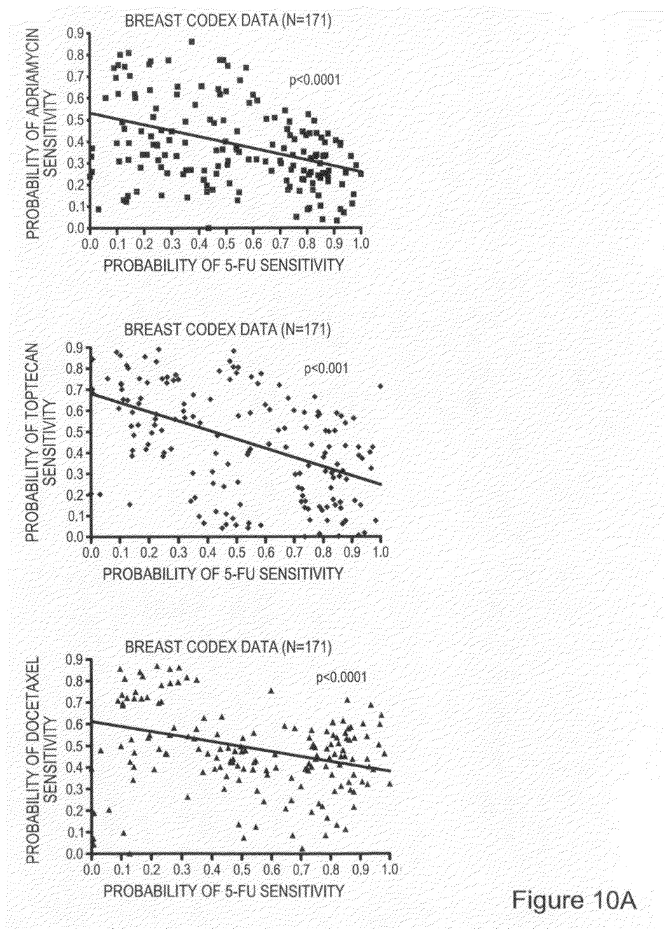

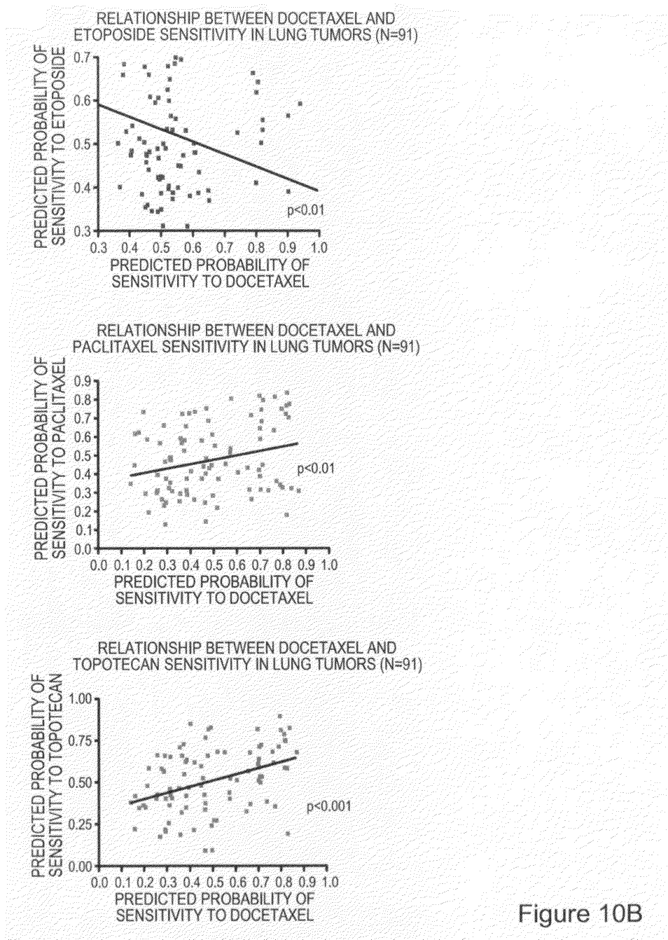

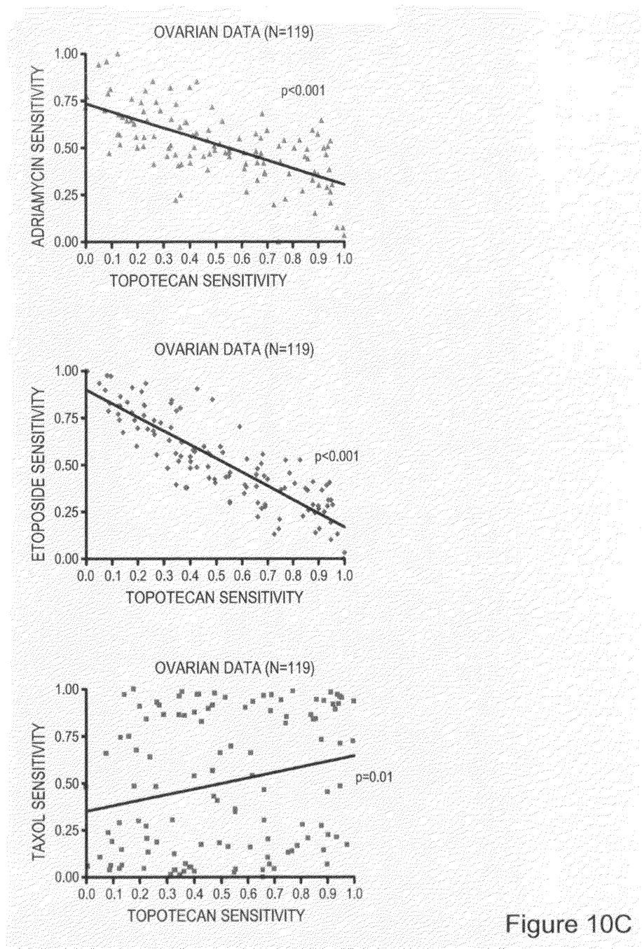

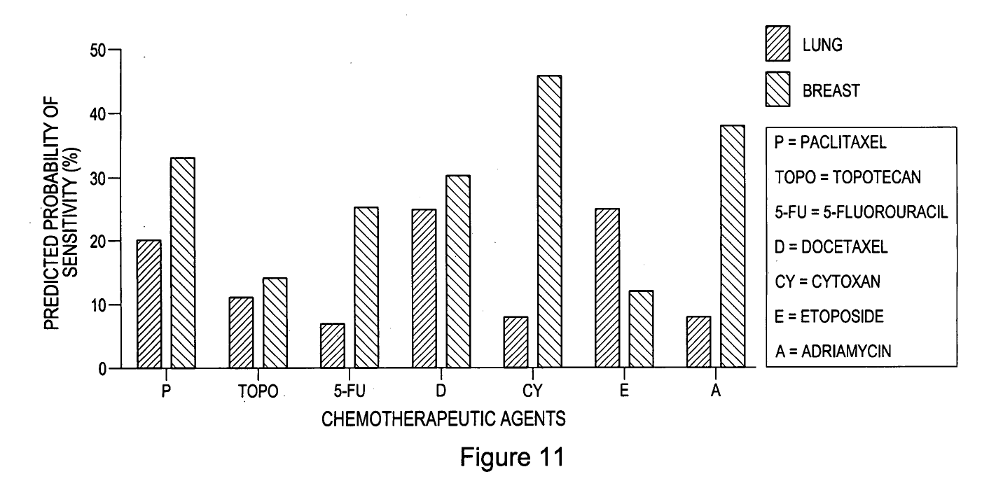

As shown in FIG. 3, there are clearly evident patterns of predicted sensitivity to the various agents. In many cases, the predicted sensitivities to the chemotherapeutic agents are consistent with the previously documented efficacy of single agent chemotherapies in the individual tumor types⁵⁷. For instance, the predicted response rate for etoposide, adriamycin, cyclophosphamide, and 5-FU approximate the observed response for these single agents in breast cancer patients (FIG. 10). Likewise, the predicted sensitivity to etoposide, docetaxel, and paclitaxel approximates the observed response for these single agents in lung cancer patients (FIG. 10). This analysis also suggests possibilities for alternate treatments. As an example, it would appear that breast cancer patients likely to respond to 5-fluorouracil are resistant to adriamycin and docetaxel (FIG. 11A). Likewise, in lung cancer, docetaxel sensitive populations are likely to be resistant to etoposide (FIG. 11B). This is a potentially useful observation considering that both etoposide and docetaxel are viable front-line options (in conjunction with cis/carboplatin) for patients with lung cancer.⁵⁸ A similar relationship is seen between topotecan and adriamycin, both agents used in salvage chemotherapy for ovarian cancer (FIG. 11C). Thus, by identifying patients/patient cohorts resistant to certain standard of care agents, one could avoid the side effects of that agent (e.g. topotecan) without compromising patient outcome, by choosing an alternative standard of care (e.g., adriamycin).

### Example 6 Linking Predictions of Chemotherapy Sensitivity to Oncogenic Pathway Deregulation

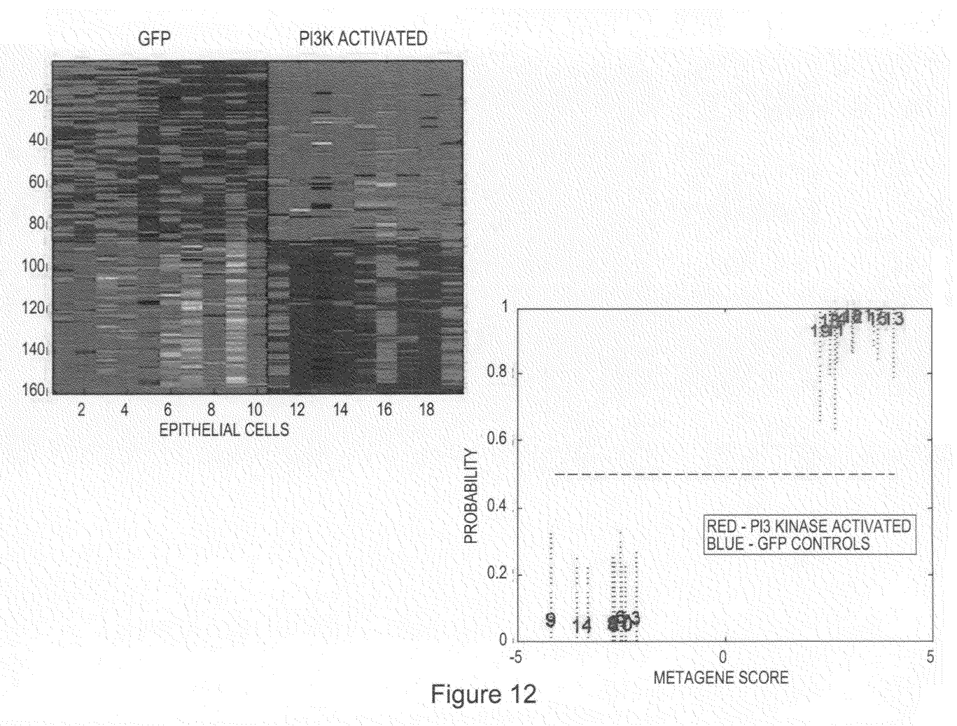

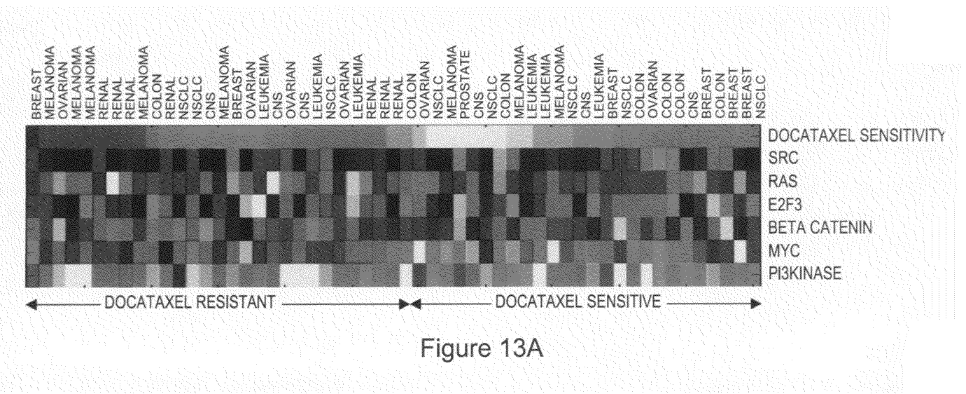

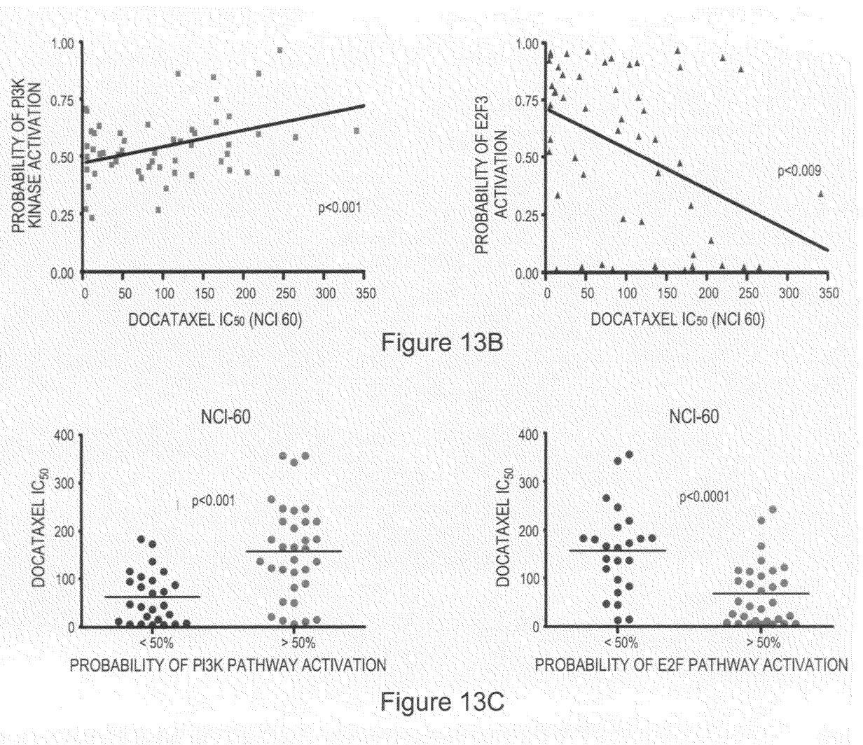

Most patients who are resistant to chemotherapeutic agents are then recruited into a second or third line therapy or enrolled to a clinical trial.^{38,59} Moreover, even those patients who initially respond to a given agent are likely to eventually suffer a relapse and in either case, additional therapeutic options are needed. As one approach to identifying such options, we have taken advantage of our recent work that describes the development of gene expression signatures that reflect the activation of several oncogenic pathways.³⁶ To illustrate the approach, we first stratified the NCI cell lines based on predicted docetaxel response and then examined the patterns of pathway deregulation associated with docetaxel sensitivity or resistance (FIG. 13A). Regression analysis revealed a significant relationship between PI3 kinase pathway deregulation and docetaxel resistance, as seen by the linear relationship (p=0.001) between the probability of PI3 kinase activation and the IC50 of docetaxel in the cell lines (FIG. 12, 28B, and Table 5).

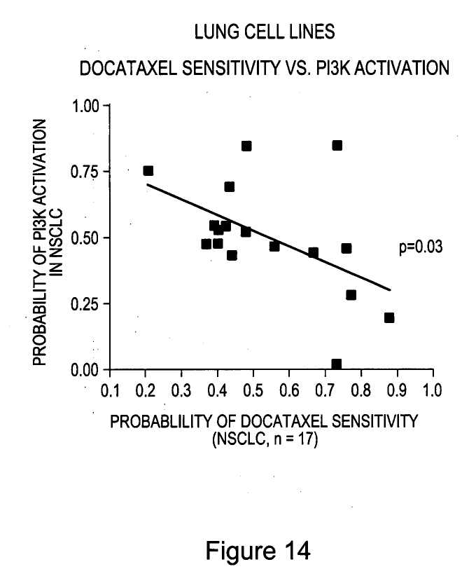

The results linking docetaxel resistance with deregulation of the PI3 kinase pathway, suggests an opportunity to employ a PI3 kinase inhibitor in this subgroup, given our recent observations that have demonstrated a linear positive correlation between the probability of pathway deregulation and targeted drug sensitivity.³⁶ To address this directly, we predicted docetaxel sensitivity and probability of oncogenic pathway deregulation using DNA microarray data from 17 NSCLC cell lines (FIG. 5A, left panel). Consistent with the analysis of the NCI-60 cell line panel, the cell lines predicted to be resistant to docetaxel were also predicted to exhibit PI3 kinase pathway activation (p=0.03, log-rank test, FIG. 14). In parallel, the lung cancer cell lines were subjected to assays for sensitivity to a PI3 kinase specific inhibitor (LY-294002), using a standard measure of cell proliferation.^{36, 38, 59} As shown by the analysis in FIG. 5B (left panel), the cell lines showing an increased probability of PI3 kinase pathway activation were also more likely to respond to a PI3 kinase inhibitor (LY-294002) (p=0.001, log-rank test)). The same relationship held for prediction of resistance to docetaxel—these cells were more likely to be sensitive to PI3 kinase inhibition (p<0.001, log-rant test) (FIG. 5B, left panel).

An analysis of a panel of ovarian cancer cell lines provided a second example. Ovarian cell lines that are predicted to be topotecan resistant (FIG. 5A, right panel) have a higher likelihood of Src pathway deregulation and there is a significant linear relationship (p=0.001, log rank) between the probability of topotecan resistance and sensitivity to a drug that inhibits the Src pathway (SU6656) (FIG. 5B, right panel). The results of these assays clearly demonstrate an opportunity to potentially mitigate drug resistance (e.g., docetaxel or topotecan) using a specific pathway-targeted agent, based on a predictor developed from pathway deregulation (i.e., PI3 kinase or Src inhibition).

Taken together, these data demonstrate an approach to the identification of therapeutic options for chemotherapy resistant patients, as well as the identification of novel combinations for chemotherapy sensitive patients, and thus represents a potential strategy to a more effective treatment plan for cancer patients, after future prospective validations trials (FIG. 6).

### Example 7 Methods

NCI-60 data. The (−log 10(M)) GI50/IC50, TGI (Total Growth Inhibition dose) and LC50 (50% cytotoxic dose) data was used to populate a matrix with MATLAB software, with the relevant expression data for the individual cell lines. Where multiple entries for a drug screen existed (by NCS number), the entry with the largest number of replicates was included. Incomplete data were assigned as Nan (not a number) for statistical purposes. To develop an in vitro gene expression based predictor of sensitivity/resistance from the pharmacologic data used in the NCI-60 drug screen studies, we chose cell lines within the NCI-60 panel that would represent the extremes of sensitivity to a given chemotherapeutic agent (mean GI50+/−1SD). Relevant expression data (updated data available on the Affymetrix U95A2 GeneChip) for the solid tumor cell lines and the respective pharmacological data for the chemotherapeutics was downloaded from the NCI website (http://dtp.nci.nih.gov/docs/cancer/cancer_data.html). The individual drug sensitivity and resistance data from the selected solid tumor NCI-60 cell lines was then used in a supervised analysis using binary regression methodologies, as described previously,⁶⁰ to develop models predictive of chemotherapeutic response.

Human ovarian cancer samples. We measured expression of 22,283 genes in 13 ovarian cancer cell lines and 119 advanced (FIGO stage III/IV) serous epithelial ovarian carcinomas using Affymetrix U133A GeneChips. All ovarian cancers were obtained at initial cytoreductive surgery from patients. All tissues were collected under the auspices of respective institutional (Duke University Medical Center and H. Lee Moffitt Cancer Center) IRB approved protocols involving written informed consent.

Full details of the methods used for RNA extraction and development of gene expression signatures representing deregulation of oncogenic pathways in the tumor samples are recently described.³⁶ Response to therapy was evaluated using standard criteria for patients with measurable disease, based upon WHO guidelines.²⁸

Lung and ovarian cancer cell culture. Total RNA was extracted and oncogenic pathway predictions was performed similar to the methods described previously.³⁶

Cross-platform Affymetrix Gene Chip comparison. To map the probe sets across various generations of Affymetrix GeneChip arrays, we utilized an in-house program, Chip Comparer (http://tenero.duhs.duke.edu/genearray/perl/chip/chipcomparer.pl) as described previously.³⁶

Cell proliferation assays. Growth curves for cells were produced by plating 500-10,000 cells per well in 96-well plates. The growth of cells at 12 hr time points (from t=12 hrs) was determined using the CellTiter 96 Aqueous One 23 Solution Cell Proliferation Assay Kit by Promega, which is a colorimetric method for determining the number of growing cells.³⁶ The growth curves plot the growth rate of cells vs. each concentration of drug tested against individual cell lines. Cumulatively, these experiments determined the concentration of cells to use for each cell line, as well as the dosing range of the inhibitors. The final dose-response curves in our experiments plot the percent of cell population responding to the chemotherapy vs. the concentration of the drug for each cell line. Sensitivity to docetaxel and a phosphatidylinositol 3-kinase (PI3 kinase) inhibitor (LY-294002)³⁶ in 17 lung cell lines, and topotecan and a Src inhibitor (SU6656) in 13 ovarian cell lines was determined by quantifying the percent reduction in growth (versus DMSO controls) at 96 hrs using a standard MTT colorimetric assay.³⁶ Concentrations used ranged from 1-10 nM for docetaxel, 300 nM-10 μM (SU6656), and 300 nM-10M for LY-294002. All experiments were repeated at least three times.

Statistical analysis methods. Analysis of expression data are as previously described.^{36, 60-62} Briefly, prior to statistical modeling, gene expression data is filtered to exclude probesets with signals present at background noise levels, and for probesets that do not vary significantly across samples. Each signature summarizes its constituent genes as a single expression profile, and is here derived as the top principal components of that set of genes. When predicting the chemosensitivity patterns or pathway activation of cancer cell lines or tumor samples, gene selection and identification is based on the training data, and then metagene values are computed using the principal components of the training data and additional cell line or tumor expression data. Bayesian fitting of binary probit regression models to the training data then permits an assessment of the relevance of the metagene signatures in within-sample classification,⁶⁰ and estimation and uncertainty assessments for the binary regression weights mapping metagenes to probabilities. To guard against over-fitting given the disproportionate number of variables to samples, we also performed leave-one-out cross validation analysis to test the stability and predictive capability of our model. Each sample was left out of the data set one at a time, the model was refitted (both the metagene factors and the partitions used) using the remaining samples, and the phenotype of the held out case was then predicted and the certainty of the classification was calculated. Given a training set of expression vectors (of values across metagenes) representing two biological states, a binary probit regression model, of predictive probabilities for each of the two states (resistant vs. sensitive) for each case is estimated using Bayesian methods. Predictions of the relative oncogenic pathway status and chemosensitivity of the validation cell lines or tumor samples are then evaluated using methods previously described^{36,60} producing estimated relative probabilities—and associated measures of uncertainty—of chemosensitivity/oncogenic pathway deregulation across the validation samples. In instances where a combined probability of sensitivity to a combination chemotherapeutic regimen was required based on the individual drug sensitivity patterns, we employed the theorem for combined probabilities as described by Feller: [Probability (Pr) of (A), (B), (C) . . . (N)= [symbol] Pr (A)+Pr (B)+Pr (C) . . . +Pr (N)−[Pr(A)×Pr(B)×Pr(C) . . . ×Pr (N)]. Hierarchical clustering of tumor predictions was performed using Gene Cluster 3.0.⁶³ Genes and tumors were clustered using average linkage with the uncentered correlation similarity metric. Standard linear regression analyses and their significance (log rank test) were generated for the drug response data and correlation between drug response and probability of chemosensitivity/pathway deregulation using GraphPad® software.

### REFERENCE BIBLIOGRAPHY

1. Levin L, Simon R, Hryniuk W: Importance of multiagent chemotherapy regimens in ovarian carcinoma: dose intensity analysis. J. Natl. Canc. Inst. 85:1732-1742, 1993
2. McGuire W P, Hoskins W J, Brady M F, et al: Assessment of dose-intensive therapy in suboptimally debulked ovarian cancer: a Gynecologic Oncology Group study. J. Clin. Oncol. 13:1589-1599, 1995
3. Jodrell D I, Egorin M J, Canetta R M, et al: Relationships between carboplatin explosure and tumor response and toxicity in patients with ovarian cancer. J. Clin. Oncol. 10:520-528, 1992
4. McGuire W P, Hoskins W J, Brady M F, et al: Cyclophosphamide and cisplatin compared with paclitaxel and cisplatin in patients with stage III and stage IV ovarian cancer. N. Engl. J. Med. 334:1-6, 1996
5. McGuire W P, Brady M F, Ozols R F: The Gynecologic Oncology Group experience in ovarian cancer. Ann. Oncol. 10:29-34, 1999
6. Piccart M J, Bertelsen K, Stuart G, et al: Long-term follow-up confirms a survival advantage of the paclitaxel-cisplatin regimen over the cyclophosphamide-cisplatin combination in advanced ovarian cancer. Int. J. Gynecol. Cancer 13:144-148, 2003
7. Wenham R M, Lancaster J M, Berchuck A: Molecular aspects of ovarian cancer. Best Pract. Res. Clin. Obstet. Gynaecol. 16:483-497, 2002
8. Berchuck A, Kohler M F, Marks J R, et al: The p53 tumor suppressor gene frequently is altered in gynecologic cancers. Am. J. Obstet. Gynecol. 170:246-252, 1994
9. Kohler M F, Marks J R, Wiseman R W, et al: Spectrum of mutation and frequency of allelic deletion of the p53 gene in ovarian cancer. J. Natl. Canc. Inst. 85:1513-1519, 1993
10. Havrilesky L, Alvarez A A, Whitaker R S, et al: Loss of expression of the p16 tumor suppressor gene is more frequent in advanced ovarian cancers lacking p53 mutations. Gynecol. Oncol. 83:491-500, 2001
11. Reles A, Wen W H, Schmider A, et al: Correlation of p53 mutations with resistance to platinum-based chemotherapy and shortened survival in ovarian cancer. Clinical Cancer Research 7:2984-2997, 2001
12. Schmider A, Gee C, Friedmann W, et al: p21 (WAF1/CIP1) protein expression is associated with prolonged survival but not with p53 expression in epithelial ovarian carcinoma. Gynecol. Oncol. 77:237-242, 2000
13. Wong K K, Cheng R S, Mok S C: Identification of differentially expressed genes from ovarian cancer cells by MICROMAX cDNA microarray system. Biotechniques 30:670-675, 2001
14. Welsh J B, Zarrinkar P P, Sapinoso L M, et al: Analysis of gene expression profiles in normal and neoplastic ovarian tissue samples identifies candidate molecular markers of epithelial ovarian cancer. Proc. Natl. Acad. Sci. USA 98:1176-1181, 2001
15. Shridhar V, Lee J-S, Pandita A, et al: Genetic analysis of early-versus late-state ovarian tumors. Cancer Res. 61:5895-5904, 2001
16. Schummer M, N g W W, Bumgarner R E, et al: Comparative hybridization of an array of 21,500 ovarian cDNAs for the discovery of genes overexpressed in ovarian carcinomas. Gene 238:375-385, 1999
17. Ono K, Tanaka T, Tsunoda T, et al: Identification by cDNA microarray of genes involved in ovarian carcinogenesis. Cancer Res. 60:5007-5011, 2000
18. Sawiris G P, Sherman-Baust C A, Becker K G, et al: Development of a highly specialized cDNA array for the study and diagnosis of epithelial ovarian cancer. Cancer Res. 62:2923-2928, 2002
19. Jazaeri A A, Yee C J, Sotiriou C, et al: Gene expression profiles of BRCA1-linked, BRCA2-linked, and sporadic ovarian cancers. J. Natl. Canc. Inst. 94:990-1000, 2002
20. Schaner M E, Ross D T, Ciaravino G, et al: Gene expression patterns in ovarian carcinomas. Mol. Biol. Cell 14:4376-4386, 2003
21. Lancaster J M, Dressman H, Whitaker R S, et al: Gene expression patterns that characterize advanced stage serous ovarian cancers. J. Surgical Gynecol. Invest. 11:51-59, 2004
22. Berchuck A, Iversen E S, Lancaster J M, et al: Patterns of gene expression that characterize long term survival in advanced serous ovarian cancers. Clin. Can. Res. 11:3686-3696, 2005
23. Berchuck A, Iversen E, Lancaster J M, et al: Prediction of optimal versus suboptimal cytoreduction of advanced stage serous ovarian cancer using microarrays. Am. J. Obstet. Gynecol. 190:910-925, 2004
24. Jazaeri A A, Awtrey C s, Chandramouli G V, et al: Gene expression profiles associated with response to chemotherapy in epithelial ovarian cancers. Clin. Cancer Res. 11:6300-6310, 2005
25. Helleman J, Jansen M P, Span P N, et al: Molecular profiling of platinum resistant ovarian cancer. Int. J. Cancer 118:1963-1971, 2005
26. Spentzos D, Levine D A, Kolia s, et al: Unique gene expression profile based on pathologic response in epithelial ovarian cancer. J. Clin. Oncol. 23:7911-7918, 2005
27. Spentzos D, Levine D A, Ramoni M F, et al: Gene expression signature with independent prognostic significance in epithelial ovarian cancer. J. Clin. Oncol. 22:4700-4710,
28. Miller A B, Hoogstraten B, Staquet M, et al: Reporting results of cancer treatment. Cancer 47:207-214, 1981
29. Rustin G J, Nelstrop A E, Bentzen S M, et al: Use of tumor markers in monitoring the course of ovarian cancer. Ann. Oncol. 10:21-27, 1999
30. Rustin G J, Nelstrop A E, McClean P, et al: Defining response of ovarian carcinoma to initial chemotherapy according to serum CA 125. J. Clin. Oncol. 14:1545-1551,
31. Irizarry R A, Hobbs B, Collin F, et al: Exploration, normalization, and summaries of high density oligonucleotide array probe level data. Biostatistics 4:249-263, 2003
32. Bolstad B M, Irizarry R A, Astrand M, et al: A comparison of normalization methods for high density oligonucleotide array data based on variance and bias. Bioinformatics 19:185-193, 2003
33. Lucus J, Carvalho C, Wang Q, et al: Sparse statistical modeling in gene expression genomics. Cambridge, Cambridge University Press, 2006
34. Rich J, Jones B, Hans C, et al: Gene expression profiling and genetic markers in glioblastoma survival. Cancer Res. 65:4051-4058, 2005
35. Hans C, Dobra A, West M: Shotgun stochastic search for regression with many candidate predictors. JASA in press., 2006
36. Bild A, Yao G, Chang J T, et al: Oncogenic pathway signatures in human cancers as a guide to targeted therapies. Nature 439:353-357, 2006.
37. Gyorrfy B, Surowiak P, Kiesslich O, Denkert C, Schafer R, Dietel M, Lage H: Gene expression profiling of 30 cancer cell lines predicts resistance towards 11 anticancer drugs at clinically achieved concentrations. Int. J. Cancer 118(7): 1699-712, 2006
38. Minna, J D, Gazdar, A F, Sprang, S R & Herz, J: Cancer. A bull's eye for targeted lung cancer therapy. Science 304: 1458-1461, 2004
39. Jemal et al., CA Cancer J. Clin., 53, 5-26, 2003
40. Cancer Facts and Figures: American Cancer Society, Atlanta, p. 11, 2002
41. Travis et al., Lung Cancer Principles and Practice, Lippincott-Raven, New York, pps. 361-395, 1996
42. Gazdar et al., Anticancer Res. 14:261-267,
43. Niklinska et al., Folia Histochem. Cytobiol. 39:147-148, 2001
44. Parker et al, CA Cancer J. Clin. 47:5-27, 1997
45. Chu et al, J. Nat. Cancer Inst. 88:1571-1579, 1996
46. Baker, V V: Salvage therapy for recurrent epithelial ovarian cancer. Hematol. Oncol. Clin. N. Am. 17: 977-988, 2003
47. Hansen, H H, Eisenhauer, E A, Hasen M, Neijt J P, Piccart M J, Sessa C, Thigpen J T: New cytostatis drugs in ovarian cancer. Ann. Oncol. 4:S63-S70, 1993.
48. Herrin, V E, Thigpen J T: Chemotherapy for ovarian cancer: current concepts. Semin. Surg. Oncol. 17:181-188, 1999
49. Staunton, J. E. et al. Chemosensitivity prediction by transcriptional profiling. Proc Natl Acad Sci USA 98:10787-19792, 2001
50. Chang, J. C. et al. Gene expression profiling for the prediction of therapeutic response to docetaxel in patients with breast cancer. Lancet 362:362-369, 2003
51. Emi, M., Kim, R., Tanabe, K., Uchida, Y. & toge, T. Targeted therapy against Bcl-2-related proteins in breast cancer cells. Breast Cancer Res 7: R940-R952, 2005
52. Takahashi, T. et al. Cyclin A-associated kinase activity is needed for paclitaxel sensitivity. Mol Cancer Ther 4:1039-1046, 2005
53. Modi, S. et al. Phosphorylated/activated HER2 as a marker of clinical resistance to single agent taxane chemotherapy for metastatic breast cancer. Cancer Invest 23: 483-487,
54. Langer, R. et al. Association of pretherapeutic expression of chemotherapy-related genes with response to neoadjuvant chemotherapy in Barrett carcinoma. Clin Cancer Res. 11: 7462-7469, 2005
55. Rouzier, R. et al. Breast cancer molecular subtypes respond differently to preoperative chemotherapy. Clin Cancer Res. 11: 5678-5685, 2005
56. Rouzier, R. et al. Microbubule-associated protein tau: a marker of paclitaxel sensitivity on breast cancer. Proc Natl Acad Sci USA 102: 8315-8320, 2005
57. DeVita, V. T., Hellman, S. & Rosenberg, S. A. Cancer. Principles and Practice of Oncology, Lippincott-Raven, Philadelphia, 2005
58. Herbst, R. S. et al. Clinical Cancer Advances 2005; Major research advances in cancer treatment, prevention, and screening—a report from the American Society of Clinical Oncology. J. Clin. Oncol. 24: 190-205, 2006
59. Broxterman, H. J. & Georgopapadakou, N. H. Anticancer therapeutics: Addictive targets, multi-targeted drugs, new drug combinations. Drug Resist Update 8:183-197, 2005
60. Pittman, J., Huang, E., Wang, Q., Nevins, J. R. & West, M. Bayesian analysis of binary prediction tree models for retrospectively sampled outcomes. Biostatistics 5: 587-601,
61. West, M. et al. Predicting the clinical status of human breast cancer by using gene expression profiles. Proc Natl Acad Sci USA 98:11462-11467, 2001
62. Ihaka, R. & Gentleman, R. A language for data analysis and graphics. J. Comput. Graph. Stat. 5: 299-314, 1996
63. Eisen, M. B., Spellman, P. T., Brown, P. O. & Botstein, D. Cluster analysis and display of genome-wide expression patterns. Proc Natl Acad Sci USA 95:14863-14868, 1998

TABLE 1
The genes constituting the individual chemosensitivity predictors.

| | | |
|---|---|---|
| Probe Set ID | Gene Title | Gene Symbol |

5-FU Predictor - Metagene 1

| | | |
|---|---|---|
| 151_s_at | “hypothetical gene LOC92755 /// tubulin, beta /// similar to | LOC92755 /// TUBB /// |
| | tubulin, beta 5” | LOC648765 |
| 1713_s_at | “cyclin-dependent kinase inhibitor 2A (melanoma, p16, inhibits | CDKN2A |
| | CDK4)” | |
| 1882_g_at | — | — |
| 31322_at | T cell receptor alpha locus | TRA@ |
| 31726_at | “gamma-aminobutyric acid (GABA) A receptor, alpha 3” | GABRA3 |
| 32308_r_at | “collagen, type I, alpha 2” | COL1A2 |
| 32318_s_at | “actin, beta” | ACTB |
| 32610_at | PDZ and LIM domain 4 | PDLIM4 |
| 32755_at | “actin, alpha 2, smooth muscle, aorta” | ACTA2 |
| 33437_at | FtsJ homolog 1 (E. coli) | FTSJ1 |
| 33444_at | neighbor of BRCA1 gene 1 /// similar to neighbor of BRCA1 | NBR1 /// LOC727732 |
| | gene 1 | |
| 33659_at | cofilin 1 (non-muscle) | CFL1 |
| 34377_at | “ATPase, Na+/K+ transporting, alpha 2 (+) polypeptide” | ATP1A2 |
| 34454_r_at | apolipoprotein C-IV | APOC4 |
| 34545_at | KIAA1509 | KIAA1509 |
| 34843_at | zinc finger protein 516 | ZNF516 |
| 34905_at | “glutamate receptor, ionotropic, kainate 5” | GRIK5 |
| 34954_r_at | “phosphodiesterase 5A, cGMP-specific” | PDE5A |
| 35056_at | arylsulfatase F | ARSF |
| 35144_at | zinc finger CCCH-type containing 7B | ZC3H7B |
| 35213_at | WW domain binding protein 4 (formin binding protein 21) | WBP4 |
| 35816_at | cystatin B (stefin B) | CSTB |
| 35929_s_at | “testis specific protein, Y-linked 1 /// testis specific protein, Y- | TSPY1 /// TSPY2 /// |
| | linked 2 /// similar to testis specific protein, Y-linked 1 /// similar | LOC653174 /// LOC728132 |
| | to testis specific protein, Y-linked 1 /// similar to testis specific | /// LOC728137 /// |
| | protein, Y-linked 1 /// similar to testis specific protein, Y-linked | LOC728395 /// LOC728403 |
| | 1 /// similar to testis specific protein, Y-linked 1 /// similar to | /// LOC728412 |
| | testis specific protein, Y-linked 1” | |
| 36245_at | 5-hydroxytryptamine (serotonin) receptor 2B | HTR2B |
| 36453_at | kelch repeat and BTB (POZ) domain containing 11 | KBTBD11 |
| 36549_at | “solute carrier family 25 (mitochondrial carrier; peroxisomal | SLC25A17 |
| | membrane protein, 34 kDa), member 17” | |
| 37349_r_at | high mobility group nucleosomal binding domain 3 | HMGN3 |
| 37361_at | fibroblast growth factor (acidic) intracellular binding protein | FIBP |
| 37437_at | intraflagellar transport 140 homolog (Chlamydomonas) | IFT140 |
| 37802_r_at | “family with sequence similarity 63, member B” | FAM63B |
| 37860_at | zinc finger protein 337 | ZNF337 |
| 39783_at | KIAA0100 | KIAA0100 |
| 39898_at | “family with sequence similarity 13, member C1” | FAM13C1 |
| 40104_at | “serine/threonine kinase 25 (STE20 homolog, yeast)” | STK25 |
| 40452_at | copine I | CPNE1 |
| 40471_at | peroxisomal biogenesis factor 19 | PEX19 |
| 40536_f_at | Eukaryotic translation initiation factor 5B | EIF5B |
| 40886_at | eukaryotic translation elongation factor 1 alpha 1 /// | EEF1A1 /// APOLD1 /// |
| | apolipoprotein L domain containing 1 /// similar to eukaryotic | LOC440595 |
| | translation elongation factor 1 alpha 1 | |
| 40983_s_at | serine racemase | SRR |
| 41058_g_at | thioesterase superfamily member 2 | THEM2 |
| 41536_at | “Inhibitor of DNA binding 4, dominant negative helix-loop-helix | ID4 |
| | protein” | |
| 41868_at | gamma-glutamyltransferase 1 /// gamma-glutamyltransferase- | GGT1 /// GGTL4 |
| | like 4 | |
| 427_f_at | “interferon, alpha 10” | IFNA10 |
| 429_f_at | “tubulin, beta 2A /// tubulin, beta 4 /// tubulin, beta 2B” | TUBB2A /// TUBB4 /// |
| | | TUBB2B |
| 471_f_at | “tubulin, beta 3” | TUBB3 |

Adriamycin Predictor - Metagene 2

| | | |
|---|---|---|
| 1051_g_at | melan-A | MLANA |
| 110_at | chondroitin sulfate proteoglycan 4 (melanoma-associated) | CSPG4 |
| 1319_at | “discoidin domain receptor family, member 2” | DDR2 |
| 1519_at | v-ets erythroblastosis virus E26 oncogene homolog 2 (avian) | ETS2 |
| 1537_at | “epidermal growth factor receptor (erythroblastic leukemia viral | EGFR |
| | (v-erb-b) oncogene homolog, avian)” | |
| 2011_s_at | BCL2-interacting killer (apoptosis-inducing) | BIK |
| 266_s_at | CD24 molecule | CD24 |
| 32139_at | zinc finger protein 185 (LIM domain) | ZNF185 |
| 32168_s_at | Down syndrome critical region gene 1 | DSCR1 |
| 32612_at | “gelsolin (amyloidosis, Finnish type)” | GSN |
| 32718_at | tyrosylprotein sulfotransferase 1 | TPST1 |
| 32821_at | lipocalin 2 (oncogene 24p3) | LCN2 |
| 32967_at | Fas apoptotic inhibitory molecule 3 | FAIM3 |
| 33004_g_at | NCK adaptor protein 2 | NCK2 |
| 33240_at | PDZ domain containing RING finger 3 | PDZRN3 |
| 33409_at | “FK506 binding protein 2, 13 kDa” | FKBP2 |
| 33824_at | keratin 8 | KRT8 |
| 33853_s_at | neuropilin 2 | NRP2 |
| 33892_at | plakophilin 2 | PKP2 |
| 33904_at | claudin 3 | CLDN3 |
| 33908_at | “calpain 1, (mu/l) large subunit” | CAPN1 |
| 33942_s_at | syntaxin binding protein 1 | STXBP1 |
| 33956_at | lymphocyte antigen 96 | LY96 |
| 34213_at | WW and C2 domain containing 1 | WWC1 |
| 34303_at | chromosome 10 open reading frame 56 | C10orf56 |
| 34348_at | “serine peptidase inhibitor, Kunitz type, 2” | SPINT2 |
| 34859_at | “melanoma antigen family D, 2” | MAGED2 |
| 34885_at | synaptogyrin 2 | SYNGR2 |
| 34993_at | “sarcoglycan, delta (35 kDa dystrophin-associated | SGCD |
| | glycoprotein)” | |
| 35280_at | “laminin, gamma 2” | LAMC2 |
| 35444_at | chromosome 19 open reading frame 21 | C19orf21 |
| 35681_r_at | zinc finger homeobox 1b | ZFHX1B |
| 35766_at | keratin 18 | KRT18 |
| 35807_at | “cytochrome b-245, alpha polypeptide” | CYBA |
| 36133_at | desmoplakin | DSP |
| 36618_g_at | “inhibitor of DNA binding 1, dominant negative helix-loop-helix | ID1 |
| | protein” | |
| 36619_r_at | “inhibitor of DNA binding 1, dominant negative helix-loop-helix | ID1 |
| | protein” | |
| 36795_at | prosaposin (variant Gaucher disease and variant | PSAP |
| | metachromatic leukodystrophy) | |
| 36828_at | zinc finger protein 629 | ZNF629 |
| 36849_at | Rho GTPase activating protein 29 | ARHGAP29 |
| 37117_at | Rho GTPase activating protein 8 /// PRR5-ARHGAP8 fusion | ARHGAP8 /// LOC553158 |
| 37251_s_at | glycoprotein M6B | GPM6B |
| 37327_at | “epidermal growth factor receptor (erythroblastic leukemia viral | EGFR |
| | (v-erb-b) oncogene homolog, avian)” | |
| 37345_at | calumenin | CALU |
| 37552_at | “potassium channel, subfamily K, member 1” | KCNK1 |
| 37695_at | ring finger protein 144 | RNF144 |
| 37743_at | fasciculation and elongation protein zeta 1 (zygin I) | FEZ1 |
| 37749_at | mesoderm specific transcript homolog (mouse) | MEST |
| 37926_at | Kruppel-like factor 5 (intestinal) | KLF5 |
| 38004_at | chondroitin sulfate proteoglycan 4 (melanoma-associated) | CSPG4 |
| 38078_at | “filamin B, beta (actin binding protein 278)” | FLNB |
| 38119_at | glycophorin C (Gerbich blood group) | GYPC |
| 38122_at | “solute carrier family 23 (nucleobase transporters), member 2” | SLC23A2 |
| 38227_at | microphthalmia-associated transcription factor | MITF |
| 38297_at | “phosphatidylinositol transfer protein, membrane-associated | PITPNM1 |
| | 1” | |
| 38379_at | glycoprotein (transmembrane) nmb | GPNMB |
| 38653_at | peripheral myelin protein 22 | PMP22 |
| 39214_at | plexin B3 /// SFRS protein kinase 3 | PLXNB3 /// SRPK3 |
| 39271_at | melanoma inhibitory activity | MIA |
| 39316_at | “RAB40C, member RAS oncogene family” | RAB40C |
| 39386_at | MAD2L1 binding protein | MAD2L1BP |
| 39801_at | “procollagen-lysine, 2-oxoglutarate 5-dioxygenase 3” | PLOD3 |
| 40103_at | villin 2 (ezrin) | VIL2 |
| 40202_at | Kruppel-like factor 9 | KLF9 |
| 40434_at | podocalyxin-like | PODXL |
| 40568_at | “ATPase, H+ transporting, lysosomal 56/58 kDa, V1 subunit | ATP6V1B2 |
| | B2” | |
| 40926_at | “solute carrier family 6 (neurotransmitter transporter, creatine), | SLC6A8 |
| | member 8” | |
| 41158_at | “proteolipid protein 1 (Pelizaeus-Merzbacher disease, spastic | PLP1 |
| | paraplegia 2, uncomplicated)” | |
| 41294_at | keratin 7 | KRT7 |
| 41359_at | plakophilin 3 | PKP3 |
| 41378_at | MRNA from chromosome 5q31-33 region | — |
| 41453_at | “discs, large homolog 3 (neuroendocrine-dlg, Drosophila)” | DLG3 |
| 41503_at | zinc fingers and homeoboxes 2 | ZHX2 |
| 41610_at | “laminin, alpha 5” | LAMA5 |
| 41644_at | SAM and SH3 domain containing 1 | SASH1 |
| 41839_at | growth arrest-specific 1 | GAS1 |
| 575_s_at | tumor-associated calcium signal transducer 1 | TACSTD1 |
| 661_at | growth arrest-specific 1 | GAS1 |
| 953_g_at | — | — |
| 999_at | “cytochrome P450, family 27, subfamily A, polypeptide 1” | CYP27A1 |

Cytotoxan Predictor - Metagene 3

| | | |
|---|---|---|
| 1356_at | death associated protein 3 | DAP3 |
| 31511_at | ribosomal protein S9 | RPS9 |
| 32252_at | “transthyretin (prealbumin, amyloidosis type I)” | TTR |
| 32318_s_at | “actin, beta” | ACTB |
| 32434_at | myristoylated alanine-rich protein kinase C substrate | MARCKS |
| 32893_s_at | gamma-glutamyltransferase 1 /// gamma-glutamyltransferase | GGT1 /// GGT2 /// GGTL4 |
| | 2 /// gamma-glutamyltransferase-like 4 /// gamma- | /// GGTLA4 /// LOC643171 |
| | glutamyltransferase-like activity 4 /// similar to Gamma- | /// LOC653590 /// |
| | glutamyltranspeptidase 1 precursor (Gamma- | LOC728226 /// LOC728441 |
| | glutamyltransferase 1) (CD224 antigen) /// similar to gamma- | /// LOC729838 /// |
| | glutamyltransferase 2 /// similar to gamma-glutamyltransferase | LOC731629 |
| | 2 /// similar to Gamma-glutamyltranspeptidase 1 precursor | |
| | (Gamma-glutamyltransferase 1) (CD224 antigen) /// similar to | |
| | gamma-glutamyltransferase-like 4 isoform 2 /// similar to | |
| | gamma-glutamyltransferase-like 4 isoform 2 | |
| 33145_at | “Fanconi anemia, complementation group A” | FANCA |
| 33362_at | CDC42 effector protein (Rho GTPase binding) 3 | CDC42EP3 |
| 33919_at | tetraspanin 4 | TSPAN4 |
| 34246_at | chromosome 6 open reading frame 145 | C6orf145 |
| 35352_at | aryl-hydrocarbon receptor nuclear translocator 2 | ARNT2 |
| 356_at | kinesin family member 22 /// similar to Kinesin-like protein | KIF22 /// LOC728037 |
| | KIF22 (Kinesin-like DNA-binding protein) (Kinesin-like protein | |
| | 4) | |
| 35763_at | neurobeachin-like 2 | NBEAL2 |
| 36119_at | “caveolin 1, caveolae protein, 22 kDa” | CAV1 |
| 36192_at | secernin 1 | SCRN1 |
| 36536_at | schwannomin interacting protein 1 | SCHIP1 |
| 37375_at | “pleckstrin homology-like domain, family B, member 1” | PHLDB1 |
| 37680_at | A kinase (PRKA) anchor protein (gravin) 12 | AKAP12 |
| 37745_s_at | suppression of tumorigenicity 5 | ST5 |
| 38288_at | snail homolog 2 (Drosophila) | SNAI2 |
| 38375_at | esterase D/formylglutathione hydrolase | ESD |
| 38479_at | “acidic (leucine-rich) nuclear phosphoprotein 32 family, | ANP32B |
| | member B” | |
| 39170_at | “CD59 molecule, complement regulatory protein” | CD59 |
| 39329_at | “actinin, alpha 1” | ACTN1 |
| 39351_at | “CD59 molecule, complement regulatory protein” | CD59 |
| 39696_at | paternally expressed 10 | PEG10 |
| 39750_at | “CDNA FLJ25106 fis, clone CBR01467” | — |
| 40213_at | “SWI/SNF related, matrix associated, actin dependent | SMARCA1 |
| | regulator of chromatin, subfamily a, member 1” | |
| 40394_at | gamma-glutamyl carboxylase | GGCX |
| 40855_at | sterile alpha motif domain containing 4A | SAMD4A |
| 40953_at | “calponin 3, acidic” | CNN3 |
| 41195_at | LIM domain containing preferred translocation partner in | LPP |
| | lipoma | |
| 41403_at | small nuclear ribonucleoprotein polypeptide F | SNRPF |
| 41449_at | “sarcoglycan, epsilon” | SGCE |
| 41739_s_at | caldesmon 1 | CALD1 |
| 41758_at | chromosome 22 open reading frame 5 | C22orf5 |

Docetaxel Predictor - Metagene 4

| | | |
|---|---|---|
| 1003_s_at | “Burkitt lymphoma receptor 1, GTP binding protein | BLR1 |
| | (chemokine (C—X—C motif) receptor 5)” | |
| 1420_s_at | “eukaryotic translation initiation factor 4A, isoform 2” | EIF4A2 |
| 1567_at | fms-related tyrosine kinase 1 (vascular endothelial growth | FLT1 |
| | factor/vascular permeability factor receptor) | |
| 1861_at | BCL2-antagonist of cell death | BAD |
| 32085_at | “phosphatidylinositol-3-phosphate/phosphatidylinositol 5- | PIP5K3 |
| | kinase, type III” | |
| 32218_at | “CDNA: FLJ22515 fis, clone HRC12122, highly similar to | — |
| | AF052101 Homo sapiens clone 23872 mRNA sequence” | |
| 32238_at | bridging integrator 1 | BIN1 |
| 32340_s_at | Y box binding protein 1 | YBX1 |
| 32828_at | branched chain ketoacid dehydrogenase kinase | BCKDK |
| 33176_at | deoxyhypusine hydroxylase/monooxygenase | DOHH |
| 33204_at | Forkhead box D1 | FOXD1 |
| 33388_at | testis expressed sequence 261 | TEX261 |
| 33444_at | neighbor of BRCA1 gene 1 /// similar to neighbor of BRCA1 | NBR1 /// LOC727732 |
| | gene 1 | |
| 34523_at | apolipoprotein A-IV | APOA4 |
| 34647_at | DEAD (Asp-Glu-Ala-Asp) box polypeptide 5 | DDX5 |
| 34773_at | tubulin folding cofactor A | TBCA |
| 34801_at | ubiquitin specific peptidase 52 | USP52 |
| 34804_at | “Solute carrier family 25, member 36” | SLC25A36 |
| 35018_at | calcium binding protein P22 | CHP |
| 35655_at | ankyrin repeat domain 28 | ANKRD28 |
| 35714_at | “pyridoxal (pyridoxine, vitamin B6) kinase” | PDXK |
| 35770_at | “ATPase, H+ transporting, lysosomal accessory protein 1” | ATP6AP1 |
| 35815_at | SET domain containing 2 | SETD2 |
| 36068_at | copper chaperone for superoxide dismutase | CCS |
| 36209_at | bromodomain containing 2 | BRD2 |
| 36250_at | aspartate beta-hydroxylase domain containing 1 | ASPHD1 |
| 36366_at | “UDP-Gal:betaGlcNAc beta 1,4-galactosyltransferase, | B4GALT6 |
| | polypeptide 6” | |
| 36395_at | Transcribed locus | — |
| 36528_at | argininosuccinate lyase | ASL |
| 36641_at | “capping protein (actin filament) muscle Z-line, alpha 2” | CAPZA2 |
| 37355_at | START domain containing 3 | STARD3 |
| 38618_at | “LIM domain kinase 2 /// protein phosphatase 1, regulatory | LIMK2 /// PPP1R14BP1 |
| | (inhibitor) subunit 14B pseudogene 1” | |
| 38663_at | barrier to autointegration factor 1 | BANF1 |
| 38831_f_at | “guanine nucleotide binding protein (G protein), beta | GNB2 |
| | polypeptide 2” | |
| 39012_g_at | endosulfine alpha | ENSA |
| 39159_at | SH3-domain GRB2-like 1 | SH3GL1 |
| 39199_at | “activin A receptor, type IB” | ACVR1B |
| 39599_at | “solute carrier family 6 (neurotransmitter transporter, GABA), | SLC6A1 |
| | member 1” | |
| 40867_at | “protein phosphatase 2 (formerly 2A), regulatory subunit A | PPP2R1A |
| | (PR 65), alpha isoform” | |
| 41063_g_at | polycomb group ring finger 1 | PCGF1 |
| 41077_at | hypothetical protein LOC643641 | LOC643641 |
| 41285_at | “inositol polyphosphate-5-phosphatase, 40 kDa” | INPP5A |
| 41489_at | “transducin-like enhancer of split 1 (E(sp1) homolog, | TLE1 |
| | Drosophila)” | |
| 41689_at | plasma membrane proteolipid (plasmolipin) | PLLP |
| 41713_at | zinc finger with KRAB and SCAN domains 1 | ZKSCAN1 |
| 41762_at | TIA1 cytotoxic granule-associated RNA binding protein-like 1 | TIAL1 |
| 910_at | “thymidine kinase 1, soluble” | TK1 |
| 922_at | “protein phosphatase 2 (formerly 2A), regulatory subunit A | PPP2R1A |
| | (PR 65), alpha isoform” | |
| 941_at | “proteasome (prosome, macropain) subunit, beta type, 6” | PSMB6 |
| 954_s_at | — | — |

Etoposide Predictor - Metagene 5

| | | |
|---|---|---|
| 1015_s_at | LIM domain kinase 1 | LIMK1 |
| 1188_g_at | “ligase III, DNA, ATP-dependent” | LIG3 |
| 1233_s_at | AXL receptor tyrosine kinase | AXL |
| 1456_s_at | “interferon, gamma-inducible protein 16” | IFI16 |
| 160020_at | matrix metallopeptidase 14 (membrane-inserted) | MMP14 |
| 1680_at | growth factor receptor-bound protein 7 | GRB7 |
| 1704_at | vav 2 oncogene | VAV2 |
| 1963_at | fms-related tyrosine kinase 1 (vascular endothelial growth | FLT1 |
| | factor/vascular permeability factor receptor) | |
| 2047_s_at | junction plakoglobin | JUP |
| 296_at | — | — |
| 297_g_at | — | — |
| 311_s_at | — | — |
| 31719_at | fibronectin 1 | FN1 |
| 31720_s_at | fibronectin 1 | FN1 |
| 32378_at | “pyruvate kinase, muscle” | PKM2 |
| 32387_at | lysophospholipase 3 (lysosomal phospholipase A2) | LYPLA3 |
| 32593_at | “raftlin, lipid raft linker 1” | RFTN1 |
| 33282_at | ladinin 1 | LAD1 |
| 33448_at | “serine peptidase inhibitor, Kunitz type 1” | SPINT1 |
| 33904_at | claudin 3 | CLDN3 |
| 34320_at | polymerase I and transcript release factor | PTRF |
| 34348_at | “serine peptidase inhibitor, Kunitz type, 2” | SPINT2 |
| 34747_at | matrix metallopeptidase 14 (membrane-inserted) | MMP14 |
| 34769_at | fatty acid amide hydrolase | FAAH |
| 35276_at | claudin 4 | CLDN4 |
| 35309_at | suppression of tumorigenicity 14 (colon carcinoma) | ST14 |
| 35444_at | chromosome 19 open reading frame 21 | C19orf21 |
| 35541_r_at | KIAA0506 protein | KIAA0506 |
| 35630_at | lethal giant larvae homolog 2 (Drosophila) /// MAP-kinase | LLGL2 /// MADD |
| | activating death domain | |
| 35669_at | cordon-bleu homolog (mouse) | COBL |
| 35681_r_at | zinc finger homeobox 1b | ZFHX1B |
| 35735_at | “guanylate binding protein 1, interferon-inducible, 67 kDa” | GBP1 |
| 36097_at | immediate early response 2 | IER2 |
| 36890_at | periplakin | PPL |
| 37934_at | transmembrane protein 30B | TMEM30B |
| 38221_at | connector enhancer of kinase suppressor of Ras 1 | CNKSR1 |
| 38482_at | claudin 7 | CLDN7 |
| 38759_at | “butyrophilin, subfamily 3, member A2” | BTN3A2 |
| 38760_f_at | “butyrophilin, subfamily 3, member A2” | BTN3A2 |
| 39331_at | “tubulin, beta 2A” | TUBB2A |
| 39732_at | microtubule-associated protein 7 | MAP7 |
| 39870_at | Testes-specific heterogenous nuclear ribonucleoprotein G-T | HNRNPG-T |
| 40215_at | UDP-glucose ceramide glucosyltransferase | UGCG |
| 40225_at | cyclin G associated kinase | GAK |
| 41359_at | plakophilin 3 | PKP3 |
| 41872_at | “deafness, autosomal dominant 5” | DFNA5 |
| 479_at | “disabled homolog 2, mitogen-responsive phosphoprotein | DAB2 |
| | (Drosophila)” | |
| 575_s_at | tumor-associated calcium signal transducer 1 | TACSTD1 |
| 671_at | “secreted protein, acidic, cysteine-rich (osteonectin)” | SPARC |
| 903_at | “protein phosphatase 2, regulatory subunit B (B56), alpha | PPP2R5A |
| | isoform” | |

Taxol Predictor - Metagene 6

| | | |
|---|---|---|
| 1218_at | nuclear receptor subfamily 2, group F, member 6 | NR2F6 |
| 1581_s_at | topoisomerase (DNA) II beta 180 kDa | TOP2B |
| 1587_at | retinoic acid receptor, gamma | RARG |
| 1824_s_at | proliferating cell nuclear antigen | PCNA |
| 1871_g_at | protein tyrosine phosphatase, non-receptor type 11 (Noonan | PTPN11 |
| | syndrome 1) | |
| 1882_g_at | — | — |
| 1903_at | — | — |
| 2001_g_at | ataxia telangiectasia mutated (includes complementation | ATM |
| | groups A, C and D) | |
| 249_at | nuclear factor of activated T-cells, cytoplasmic, calcineurin- | NFATC4 |
| | dependent 4 | |
| 32386_at | MRNA full length insert cDNA clone EUROIMAGE 117929 | — |
| 33064_at | calcium channel, voltage-dependent, gamma subunit 1 | CACNG1 |
| 33557_at | chromosome 22 open reading frame 31 | C22orf31 |
| 335_r_at | — | — |
| 34197_at | phosphoinositide-3-kinase, regulatory subunit 2 (p85 beta) | PIK3R2 |
| 34247_at | Protease, serine, 12 (neurotrypsin, motopsin) | PRSS12 |
| 34471_at | myosin, heavy chain 8, skeletal muscle, perinatal | MYH8 |
| 34862_at | saccharopine dehydrogenase (putative) | SCCPDH |
| 34909_at | putative homeodomain transcription factor 2 | PHTF2 |
| 34923_at | IQ motif and Sec7 domain 2 | IQSEC2 |
| 34984_at | transient receptor potential cation channel, subfamily C, | TRPC3 |
| | member 3 | |
| 35254_at | TRAF-type zinc finger domain containing 1 | TRAFD1 |
| 35644_at | hephaestin | HEPH |
| 35908_at | SRY (sex determining region Y)-box 30 | SOX30 |
| 36595_s_at | glycine amidinotransferase (L-arginine:glycine | GATM |
| | amidinotransferase) | |
| 37378_r_at | lamin A/C | LMNA |
| 37767_at | huntingtin (Huntington disease) | HD |
| 38680_at | — | — |
| 38697_at | Yip1 domain family, member 3 | YIPF3 |
| 38703_at | aspartyl aminopeptidase | DNPEP |
| 39488_at | Protocadherin 9 | PCDH9 |
| 39537_at | kelch domain containing 3 | KLHDC3 |
| 40360_at | solute carrier family 10 (sodium/bile acid cotransporter family), | SLC10A3 |
| | member 3 | |
| 40529_at | LIM homeobox 2 | LHX2 |
| 40690_at | CDC28 protein kinase regulatory subunit 2 | CKS2 |
| 41045_at | secreted and transmembrane 1 | SECTM1 |
| 41204_s_at | splicing factor 1 | SF1 |
| 41404_at | ribosomal protein S6 kinase, 90 kDa, polypeptide 4 | RPS6KA4 |
| 761_g_at | dual-specificity tyrosine-(Y)-phosphorylation regulated kinase 2 | DYRK2 |
| 777_at | GDP dissociation inhibitor 2 | GDI2 |
| 925_at | interferon, gamma-inducible protein 30 | IFI30 |

Topotecan Predictor - Metagene 7

| | | |
|---|---|---|
| 1005_at | dual specificity phosphatase 1 | DUSP1 |
| 115_at | thrombospondin 1 | THBS1 |
| 1233_s_at | AXL receptor tyrosine kinase | AXL |
| 1251_g_at | RAP1 GTPase activating protein | RAP1GAP |
| 1257_s_at | quiescin Q6 | QSCN6 |
| 1278_at | — | — |
| 1368_at | “interleukin 1 receptor, type I” | IL1R1 |
| 1385_at | “transforming growth factor, beta-induced, 68 kDa” | TGFBI |
| 1491_at | “pentraxin-related gene, rapidly induced by IL-1 beta” | PTX3 |
| 1544_at | Bloom syndrome | BLM |
| 1563_s_at | “tumor necrosis factor receptor superfamily, member 1A” | TNFRSF1A |
| 1593_at | fibroblast growth factor 2 (basic) | FGF2 |
| 159_at | vascular endothelial growth factor C | VEGFC |
| 160044_g_at | “aconitase 2, mitochondrial” | ACO2 |
| 1751_g_at | “phenylalanine-tRNA synthetase-like, alpha subunit” | FARSLA |
| 1783_at | Ras and Rab interactor 2 | RIN2 |
| 1828_s_at | fibroblast growth factor 2 (basic) | FGF2 |
| 1879_at | related RAS viral (r-ras) oncogene homolog | RRAS |
| 1958_at | c-fos induced growth factor (vascular endothelial growth factor | FIGF |
| | D) | |
| 2042_s_at | v-myb myeloblastosis viral oncogene homolog (avian) | MYB |
| 2053_at | “cadherin 2, type 1, N-cadherin (neuronal)” | CDH2 |
| 2056_at | “fibroblast growth factor receptor 1 (fms-related tyrosine | FGFR1 |
| | kinase 2, Pfeiffer syndrome)” | |
| 2057_g_at | “fibroblast growth factor receptor 1 (fms-related tyrosine | FGFR1 |
| | kinase 2, Pfeiffer syndrome)” | |
| 232_at | “laminin, gamma 1 (formerly LAMB2)” | LAMC1 |
| 31521_f_at | “histone cluster 1, H4k /// histone cluster 1, H4j” | HIST1H4K /// HIST1H4J |
| 32098_at | “collagen, type VI, alpha 2” | COL6A2 |
| 32116_at | transmembrane channel-like 6 | TMC6 |
| 32260_at | phosphoprotein enriched in astrocytes 15 | PEA15 |
| 32434_at | myristoylated alanine-rich protein kinase C substrate | MARCKS |
| 32529_at | cytoskeleton-associated protein 4 | CKAP4 |
| 32531_at | “gap junction protein, alpha 1, 43 kDa (connexin 43)” | GJA1 |
| 32535_at | fibrillin 1 | FBN1 |
| 32606_at | “Brain abundant, membrane attached signal protein 1” | BASP1 |
| 32607_at | “brain abundant, membrane attached signal protein 1” | BASP1 |
| 32673_at | “butyrophilin, subfamily 2, member A1” | BTN2A1 |
| 32808_at | “integrin, beta 1 (fibronectin receptor, beta polypeptide, | ITGB1 |
| | antigen CD29 includes MDF2, MSK12)” | |
| 32812_at | hypothetical protein | DKFZP686A01247 |
| 32847_at | “myosin, light chain kinase” | MYLK |
| 33127_at | lysyl oxidase-like 2 | LOXL2 |
| 33328_at | HEG homolog 1 (zebrafish) | HEG1 |
| 33337_at | “degenerative spermatocyte homolog 1, lipid desaturase | DEGS1 |
| | (Drosophila)” | |
| 33404_at | “CAP, adenylate cyclase-associated protein, 2 (yeast)” | CAP2 |
| 33405_at | “CAP, adenylate cyclase-associated protein, 2 (yeast)” | CAP2 |
| 33440_at | — | — |
| 33772_at | prostaglandin E receptor 4 (subtype EP4) | PTGER4 |
| 33785_at | brain-specific angiogenesis inhibitor 2 | BAI2 |
| 33787_at | “NUAK family, SNF1-like kinase, 1” | NUAK1 |
| 33791_at | “deleted in lymphocytic leukemia, 1 /// SPANX family, member | DLEU1 /// SPANXC |
| | C” | |
| 33882_at | RAB11 family interacting protein 5 (class I) | RAB11FIP5 |
| 33900_at | follistatin-like 3 (secreted glycoprotein) | FSTL3 |
| 33994_g_at | “myosin, light chain 6, alkali, smooth muscle and non-muscle” | MYL6 |
| 34091_s_at | vimentin | VIM |
| 34106_at | guanine nucleotide binding protein (G protein) alpha 12 | GNA12 |
| 34318_at | “PRA1 domain family, member 2” | PRAF2 |
| 34320_at | polymerase I and transcript release factor | PTRF |
| 34375_at | chemokine (C-C motif) ligand 2 | CCL2 |
| 34795_at | “procollagen-lysine, 2-oxoglutarate 5-dioxygenase 2” | PLOD2 |
| 34802_at | “collagen, type VI, alpha 2” | COL6A2 |
| 34811_at | “ATP synthase, H+ transporting, mitochondrial F0 complex, | ATP5G3 |
| | subunit C3 (subunit 9)” | |
| 35130_at | glutathione reductase | GSR |
| 35264_at | “NADH dehydrogenase (ubiquinone) Fe—S protein 3, 30 kDa | NDUFS3 |
| | (NADH-coenzyme Q reductase)” | |
| 35309_at | suppression of tumorigenicity 14 (colon carcinoma) | ST14 |
| 35366_at | nidogen 1 | NID1 |
| 35729_at | myosin ID | MYO1D |
| 35751_at | “succinate dehydrogenase complex, subunit B, iron sulfur (Ip)” | SDHB |
| 36119_at | “caveolin 1, caveolae protein, 22 kDa” | CAV1 |
| 36149_at | dihydropyrimidinase-like 3 | DPYSL3 |
| 36369_at | polymerase I and transcript release factor | PTRF |
| 36525_at | F-box and leucine-rich repeat protein 2 | FBXL2 |
| 36550_at | Ras and Rab interactor 2 | RIN2 |
| 36577_at | “pleckstrin homology domain containing, family C (with FERM | PLEKHC1 |
| | domain) member 1” | |
| 36638_at | connective tissue growth factor | CTGF |
| 36659_at | “collagen, type IV, alpha 2” | COL4A2 |
| 36790_at | tropomyosin 1 (alpha) | TPM1 |
| 36791_g_at | tropomyosin 1 (alpha) | TPM1 |
| 36792_at | tropomyosin 1 (alpha) | TPM1 |
| 36799_at | frizzled homolog 2 (Drosophila) | FZD2 |
| 36811_at | lysyl oxidase-like 1 | LOXL1 |
| 36885_at | spleen tyrosine kinase | SYK |
| 36952_at | “hydroxyacyl-Coenzyme A dehydrogenase/3-ketoacyl- | HADHA |
| | Coenzyme A thiolase/enoyl-Coenzyme A hydratase | |
| | (trifunctional protein), alpha subunit” | |
| 36988_at | “tumor necrosis factor, alpha-induced protein 1 (endothelial)” | TNFAIP1 |
| 37032_at | nicotinamide N-methyltransferase | NNMT |
| 37322_s_at | hydroxyprostaglandin dehydrogenase 15-(NAD) | HPGD |
| 37408_at | “mannose receptor, C type 2” | MRC2 |
| 37486_f_at | Meis1 homolog 3 (mouse) pseudogene 1 | MEIS3P1 |
| 37599_at | aldehyde oxidase 1 | AOX1 |
| 376_at | “sema domain, immunoglobulin domain (Ig), short basic | SEMA3C |
| | domain, secreted, (semaphorin) 3C” | |
| 377_g_at | “sema domain, immunoglobulin domain (Ig), short basic | SEMA3C |
| | domain, secreted, (semaphorin) 3C” | |
| 38113_at | “spectrin repeat containing, nuclear envelope 1” | SYNE1 |
| 38125_at | “serpin peptidase inhibitor, clade E (nexin, plasminogen | SERPINE1 |
| | activator inhibitor type 1), member 1” | |
| 38299_at | “interleukin 6 (interferon, beta 2)” | IL6 |
| 38338_at | related RAS viral (r-ras) oncogene homolog | RRAS |
| 38394_at | glycerol-3-phosphate dehydrogenase 1-like | GPD1L |
| 38396_at | 3′UTR of hypothetical protein (ORF1) | — |
| 38433_at | AXL receptor tyrosine kinase | AXL |
| 38449_at | WD repeat domain 23 | WDR23 |
| 38482_at | claudin 7 | CLDN7 |
| 38488_s_at | interleukin 15 | IL15 |
| 38631_at | “tumor necrosis factor, alpha-induced protein 2” | TNFAIP2 |
| 38772_at | “cysteine-rich, angiogenic inducer, 61” | CYR61 |
| 38775_at | low density lipoprotein-related protein 1 (alpha-2- | LRP1 |
| | macroglobulin receptor) | |
| 38842_at | angiomotin like 2 | AMOTL2 |
| 38921_at | “phosphodiesterase 1B, calmodulin-dependent” | PDE1B |
| 39100_at | “sparc/osteonectin, cwcv and kazal-like domains proteoglycan | SPOCK1 |
| | (testican) 1” | |
| 39254_at | retinoic acid induced 14 | RAI14 |
| 39277_at | — | — |
| 39327_at | peroxidasin homolog (Drosophila) | PXDN |
| 39333_at | “collagen, type IV, alpha 1” | COL4A1 |
| 39409_at | “complement component 1, r subcomponent” | C1R |
| 39614_at | KIAA0802 /// chromosome 21 open reading frame 57 | KIAA0802 /// C21orf57 |
| 39710_at | chromosome 5 open reading frame 13 | C5orf13 |
| 39867_at | “Tu translation elongation factor, mitochondrial” | TUFM |
| 39901_at | EGF-like repeats and discoidin I-like domains 3 | EDIL3 |
| 40023_at | brain-derived neurotrophic factor | BDNF |
| 40078_at | “protease, serine, 23” | PRSS23 |
| 40096_at | “ATP synthase, H+ transporting, mitochondrial F1 complex, | ATP5A1 |
| | alpha subunit 1, cardiac muscle” | |
| 40171_at | frequently rearranged in advanced T-cell lymphomas 2 | FRAT2 |
| 40341_at | chromosome 16 open reading frame 51 | C16orf51 |
| 40497_at | tumor suppressor candidate 4 | TUSC4 |
| 40564_at | nucleoporin 50 kDa | NUP50 |
| 40567_at | “tubulin, alpha 3” | TUBA3 |
| 40642_at | nuclear factor I/B | NFIB |
| 40692_at | “transducin-like enhancer of split 4 (E(sp1) homolog, | TLE4 |
| | Drosophila)” | |
| 40781_at | “V-akt murine thymoma viral oncogene homolog 3 (protein | AKT3 |
| | kinase B, gamma)” | |
| 40936_at | cysteine rich transmembrane BMP regulator 1 (chordin-like) | CRIM1 |
| 41197_at | RAD23 homolog A (S. cerevisiae) | RAD23A |
| 41223_at | cytochrome c oxidase subunit Va | COX5A |
| 41236_at | “Smith-Magenis syndrome chromosome region, candidate 7- | SMCR7L |
| | like” | |
| 41273_at | matrix-remodelling associated 7 | MXRA7 |
| 41295_at | START domain containing 7 | STARD7 |
| 41354_at | stanniocalcin 1 | STC1 |
| 41478_at | tetratricopeptide repeat domain 28 | TTC28 |
| 41544_at | polo-like kinase 2 (Drosophila) | PLK2 |
| 41667_s_at | “TDP-glucose 4,6-dehydratase” | TGDS |
| 41738_at | caldesmon 1 | CALD1 |
| 41744_at | optineurin | OPTN |
| 41745_at | interferon induced transmembrane protein 3 (1-8U) | IFITM3 |
| 41872_at | “deafness, autosomal dominant 5” | DFNA5 |
| 424_s_at | “fibroblast growth factor receptor 1 (fms-related tyrosine | FGFR1 |
| | kinase 2, Pfeiffer syndrome)” | |
| 465_at | “HIV-1 Tat interacting protein, 60 kDa” | HTATIP |
| 548_s_at | spleen tyrosine kinase | SYK |
| 581_at | “laminin, beta 1” | LAMB1 |
| 628_at | frizzled homolog 2 (Drosophila) | FZD2 |
| 672_at | “serpin peptidase inhibitor, clade E (nexin, plasminogen | SERPINE1 |
| | activator inhibitor type 1), member 1” | |
| 867_s_at | thrombospondin 1 | THBS1 |
| 875_g_at | chemokine (C-C motif) ligand 2 | CCL2 |
| 884_at | “integrin, alpha 3 (antigen CD49C, alpha 3 subunit of VLA-3 | ITGA3 |
| | receptor)” | |
| 885_g_at | “integrin, alpha 3 (antigen CD49C, alpha 3 subunit of VLA-3 | ITGA3 |
| | receptor)” | |
| 890_at | ubiquitin-conjugating enzyme E2A (RAD6 homolog) | UBE2A |
| 919_at | — | — |

TABLE 2

| | | | Genomic-based | |
|---|---|---|---|---|
| | Actual | | Prediction of Response | |
| Tumor data set/Response | Overall response | | (i.e. PPV for Response) | |
| Breast Tumor Data | | | | |
| MDACC | 13/51 | (25.4%) | 11/13 | (85.7%) |
| Adjuvant | 33/45 | (66.6%) | 28/31 | (90.3%) |
| Neoadjuvant Docetaxel | 13/24 | (54.1%) | 11/13 | (85.7%) |
| Ovarian | | | | |
| Topotecan | 20/48 | (41.6%) | 17/22 | (77.3%) |
| Paclitaxel | 20/35 | (57.1%) | 20/28 | (71.5%) |
| Docetaxel | 7/14 | (50%) | 6/7 | (85.7%) |
| Adriamycin (Evans et al) | 24/122 | (19.6%) | 19/33 | (57.5%) |

TABLE 3

| | Drugs | | | | | | | |
|---|---|---|---|---|---|---|---|---|
| Validations | Topotecan | Adriamycin | Etoposide | 5-Flourouracil | Paclitaxel | Cytoxan | Docetaxel | |
| In vitro Data | | | | | | | | |
| Accuracy | 18/20 (90%) | 18/25 (86%) | 21/24 (87%) | 21/24 (87%) | 26/28 (92.8%) | 25/29 (86.2%) | P < 0.001** | |
| PPV | 12/14 (86%) | 13/13 (100%) | 6/8 (75%) | 14/14 (100%) | 21/21 (100%) | 13/15 (86.6%) | | |
| NPV | 6/6 (100%) | 5/8 (62.5%) | 15/16 (94%) | 7/10 (70%) | 5/7 (71.5%) | 12/14 (86%) | | |
| In vivo | | | | | | | | |
| (Patient) Data | | | | | | | Breast | Ovarian |
| Accuracy | 40/48 (83.32%) | 99/122 (81%) | — | — | 28/35 (80%) | — | 22/24 (91.6%) | 12/14 (85.7%) |
| PPV | 17/22 (77.34%) | 19/33 (57.5%) | | | 20/28 (71.4%) | | 11/13 (85.7%) | 6/7 (85.7%) |
| NPV | 23/26 (88.5%) | 80/89 (89.8%) | | | 7/7 (100%) | | 11/11 (100%) | 6/7 (85.7%) |

PPV—positive predictive value,
NPV—negative predictive value.
**Determining accuracy for the docetaxel predictor in the IJC cell line data set was not possible since docetaxel was not one of the drugs studied. Instead, the docetaxel predictor was validated in two independent cell line experiments, correlating predicted probability of response to docetaxel in vitro with actual IC50 of docetaxel by cell line (FIG. 1C).

TABLE 4

| | Predictors | | | |
|---|---|---|---|---|
| | | | Genomic predictor of response to | Predictor of response to |
| | Docetaxel predictor | Docetaxel predictor | TFAC chemotherapy | TFAC chemotherapy |
| Validations | (Potti et al) | (Chang et al)** | (Potti et al) | (Pusztai et al)** |
| Breast neoadjuvant data | | | | |
| (Chang et al) | | | | |
| Accuracy | 22/24 (91.6%) | 87.5% | | |
| PPV | 11/13 (85.7%) | 92% | | |
| NPV | 11/11 (100%) | 83% | | |
| AUC of ROC | 0.97 | 0.96 | | |
| MDACC data (Pusztai et al) | | | | |
| Accuracy | | | 42/51 (82.3%) | 74% |
| PPV | | | 11/18 (61.1%) | 44% |
| NPV | | | 31/33 (94%) | 93% |

PPV—positive predictive value.
NPV—negative predictive value.
**For both the Chang and Pusztai data, the actual numbers of predicted responders was not available, just the predictive accuracies. Also, the predictive accuracy reported for the Chang data is not in an independent validation, instead it is for a leave-one out cross validation.

TABLE 5
Genes constituting the PI3 kinase predictor

| Gene Symbol | Affymetrix Probe ID | Gene Title |
|---|---|---|
| RFC2 | 1053_at | replication factor C (activator 1) 2, 40 kDa |
| KIAA0153 | 1552257_a_at | KIAA0153 protein |
| EXOSC6 | 1553947_at | exosome component 6 |
| RHOB | 1553962_s_at | ras homolog gene family, member B |
| MAD2L1 | 1554768_a_at | MAD2 mitotic arrest deficient-like 1 (yeast) |
| RBM15 | 1555762_s_at | RNA binding motif protein 15 |
| SPEN | 1556059_s_at | spen homolog, transcriptional regulator (Drosophila) |
| C6orf150 | 1559051_s_at | chromosome 6 open reading frame 150 |
| HSPA1A | 200799_at | heat shock 70 kDa protein 1A |
| HSPA1A /// HSPA1B | 200800_s_at | heat ahock 70 kDa protein 1A /// heat shock 70 kDa protein 1B |
| NOL5A | 200875_s_at | nucleolar protein 5A (56 kDa with KKE/D repeat) |
| CSE1L | 201112_s_at | CSE1 chromosome segregation 1-like (yeast) |
| PCNA | 201202_at | proliferating cell nuclear antigen |
| JUN | 201464_x_at | v-jun sarcoma virus 17 oncogene homolog (avian) |
| JUN | 201465_s_at | v-jun sarcoma virus 17 oncogene homolog (avian) |
| JUN | 201466_s_at | v-jun sarcoma virus 17 oncogene homolog (avian) |
| JUNB | 201473_at | jun B proto-oncogene |
| MCM3 | 201555_at | MCM3 minichromosome maintenance deficient 3 (S. cerevisiae) |
| EGR1 | 201693_s_at | early growth response 1 |
| DNMT1 | 201697_s_at | DNA (cytosine-5-)-methyltransferase 1 |
| MCM5 | 201755_at | MCM5 minichromosome maintenance deficient 5, cell division cycle 46 (S. cerevisiae) |
| RRM2 | 201890_at | ribonucleotide reductase M2 polypeptide |
| MCM6 | 201930_at | MCM6 minichromosome maintenance deficient 6 (MIS5 homolog, S. pombe) (S. cerevisiae) |
| NASP | 201970_s_at | nuclear autoantigenic sperm protein (histone-binding) |
| SPEN | 201997_s_at | spen homolog, transcriptional regulator (Drosophila) |
| IER2 | 202081_at | immediate early response 2 |
| MCM2 | 202107_s_at | MCM2 minichromosome maintenance deficient 2, mitotin (S. cerevisise) |
| MTHFD1 | 202309_at | methylenetetrahydrofolate dehydrogenase (NADP+ dependent) 1, methenyltetrahydrofolate |
| | | cyclohydrolase, formyltetrahydrofolate synthetase |
| UNG | 202330_s_at | uracil-DNA glycosylase |
| HSPA1B | 202581_at | heat shock 70 kDa protein 1B |
| MSH6 | 202911_at | mutS homolog 6 (E. coli) |
| SSX2IP | 203017_s_at | synovial sarcoma, X breakpoint 2 interacting protein |
| RNASEH2A | 203022_at | ribonuclease H2, large subunit |
| PEX5 | 203244_at | peroxisomal biogenesis factor 5 |
| LMNB1 | 203276_at | lamin B1 |
| POLD1 | 203422_at | polymerase (DNA directed), delta 1, catalytic subunit 125 kDa |
| CDC6 | 203968_s_at | CDC6 cell division cycle 6 homolog (S. cerevisiae) |
| ZWINT | 204026_s_at | ZW10 interactor |
| CDC45L | 204126_s_at | CDC45 cell division cycle 45-like (S. cerevisiae) |
| RFC3 | 204128_s_at | replication factor C (activator 1) 3, 38 kDa |
| POLA2 | 204441_s_at | polymerase (DNA directed), alpha 2 (70 kD subunit) |
| CDC7 | 204510_at | CDC7 cell division cycle 7 (S. cerevisiae) |
| DIPA | 204610_s_at | hepatitis delta antigen-interacting protein A |
| ACD | 204617_s_at | adrenocortical dysplasia homolog (mouse) |
| CDC25A | 204695_at | cell division cycle 25A |
| FEN1 | 204767_s_at | flap structure-specific endonuclease 1 |
| FEN1 | 204768_s_at | flap structure-specific endonuclease 1 |
| MYB | 204798_at | v-myb myeloblastosis viral oncogene homolog (avian) |
| TOP3A | 204946_s_at | topoisomerase (DNA) III alpha |
| DDX10 | 204977_at | DEAD (Asp-Glu-Ala-Asp) box polypeptide 10 |
| RAD51 | 205024_s_at | RAD51 homolog (RecA homolog, E. coli) (S. cerevisiae) |
| CCNE2 | 205034_at | cyclin E2 |
| PRIM1 | 205053_at | primase, polypeptide 1, 49 kDa |
| BARD1 | 205345_at | BRCA1 associated RING domain 1 |
| CHEK1 | 205393_s_at | CHK1 checkpoint homolog (S. pombe) |
| H2AFX | 205436_s_at | H2A histone family, member X |
| FLJ12973 | 205519_at | hypothetical protein FLJ12973 |
| GEMIN4 | 205527_s_at | gem (nuclear organelle) associated protein 4 |
| SLBP | 206052_s_at | stem-loop (histone) binding protein |
| KIAA0186 | 206102_at | KIAA0186 gene product |
| AKR7A3 | 206469_x_at | aldo-keto reductase family 7, member A3 (aflatoxin aldehyde reductase) |
| TLE3 | 206472_s_at | transducin-like enhancer of split 3 (E(sp1) homolog, Drosophila) |
| GADD45B | 207574_s_at | growth arrest and DNA-damage-inducible, beta |
| PRPS1 | 208447_s_at | phosphoribosyl pyrophosphate synthetase 1 |
| BRD2 | 208685_x_at | bromodomain containing 2 |
| BRD2 | 208686_s_at | bromodomain containing 2 |
| MCM7 | 208795_s_at | MCM7 minichromosome maintenance deficient 7 (S. cerevisiae) |
| ID1 | 208937_s_at | inhibitor of DNA binding 1, dominant negative helix-loop-helix protein |
| GADD45B | 209304_x_at | growth arrest and DNA-damage-inducible, beta |
| GADD45B | 209305_s_at | growth arrest and DNA-damage-inducible, beta |
| POLR1C | 209317_at | polymerase (RNA) I polypeptide C, 30 kDa |
| PRKRIR | 209323_at | protein-kinase, interferon-inducible double stranded RNA dependent inhibitor, repressor of |
| | | (P58 repressor) |
| MSH2 | 209421_at | mutS homolog 2, colon cancer, nonpolyposis type 1 (E. coli) |
| PPAT | 209433_s_at | phosphoribosyl pyrophosphate amidotransferase |
| PPAT | 209434_s_at | phosphoribosyl pyrophosphate amidotransferase |
| PRPS1 | 209440_at | phosphoribosyl pyrophosphate synthetase 1 |
| RPA3 | 209507_at | replication protein A3, 14 kDa |
| EED | 209572_s_at | embryonic ectoderm development |
| GAS2L1 | 209729_at | growth arrest-specific 2 like 1 |
| RRM2 | 209773_s_at | ribonucleotide reductase M2 polypeptide |
| SLC19A1 | 209777_s_at | solute carrier family 19 (folate transporter), member 1 |
| CDT1 | 209832_s_at | DNA replication factor |
| SHMT1 | 209980_s_at | serine hydroxymethyltransferase 1 (soluble) |
| TAF5 | 210053_at | TAF5 RNA polymerase II, TATA box binding protein (TBP)-associated factor, 100 kDa |
| MCM7 | 210983_s_at | MCM7 minichromosome maintenance deficient 7 (S. cerevisiae) |
| MSH6 | 211450_s_at | mutS homolog 6 (E. coli) |
| CCNE2 | 211814_s_at | cyclin E2 |
| RHOB | 212099_at | ras homolog gene family, member B |
| MCM4 | 212141_at | MCM4 minichromosome maintenance deficient 4 (S. cerevisiae) |
| MCM4 | 212142_at | MCM4 minichromosome maintenance deficient 4 (S. cerevisiae) |
| KCTD12 | 212188_at | potassium channel tetramerisation domain containing 12 /// potassium channel tetramerisation |
| | | domain containing 12 |
| KCTD12 | 212192_at | potassium channel tetramerisation domain containing 12 |
| MAC30 | 212281_s_at | hypothetical protein MAC30 |
| POLD3 | 212836_at | polymerase (DNA-directed), delta 3, accessory subunit |
| KIAA0406 | 212898_at | KIAA0406 gene product |
| FLJ10719 | 213007_at | hypothetical protein FLJ10719 |
| ITPKC | 213076_at | inositol 1,4,5-trisphosphate 3-kinase C |
| ZNF473 | 213124_at | zinc finger protein 473 |
| — | 213281_at | — |
| CCNE1 | 213523_at | cyclin E1 |
| GADD45B | 213560_at | Growth arrest and DNA-damage-inducible, beta |
| GAL | 214240_at | galanin |
| BRD2 | 214911_s_at | bromodomain containing 2 |
| UMPS | 215165_x_at | uridine monophosphate synthetase (orotate phosphoribosyl transferase and orotidine-5′-decarboxylase) |
| MCM5 | 216237_s_at | MCM5 minichromosome maintenance deficient 5, cell division cycle 46 (S. cerevisiae) |
| LMNB2 | 216952_s_at | lamin B2 |
| GEMIN4 | 217099_s_at | gem (nuclear organelle) associated protein 4 |
| SUPT16H | 217815_at | suppressor of Ty 16 homolog (S. cerevisiae) |
| GMNN | 218350_s_at | geminin, DNA replication inhibitor |
| RAMP | 218585_s_at | RA-regulated nuclear matrix-associated protein |
| SLC25A15 | 218653_at | solute carrier family 25 (mitochondrial carrier; ornithine transporter) member 15 |
| FLJ13912 | 218719_s_at | hypothetical protein FLJ13912 |
| ATAD2 | 218782_s_at | ATPase family, AAA domain containing 2 |
| C10orf117 | 218889_at | chromosome 10 open reading frame 117 |
| MGC10993 | 218897_at | hypothetical protein MGC10993 |
| C21orf45 | 219004_s_at | chromosome 21 open reading frame 45 |
| RPP25 | 219143_s_at | ribonuclease P 25 kDa subunit |
| FLJ20516 | 219258_at | timeless-interacting protein |
| MGC4504 | 219270_at | hypothetical protein MGC4504 |
| RBM15 | 219286_s_at | RNA binding motif protein 15 |
| FLJ11078 | 219354_at | hypothetical protein FLJ11078 |
| DCLRE1B | 219490_s_at | DNA cross-link repair 1B (PSO2 homolog, S. cerevisiae) |
| FLJ34077 | 219731_at | weakly similar to zinc finger protein 195 |
| FLJ20257 | 219798_s_at | hypothetical protein FLJ20257 |
| MCM10 | 220651_s_t | MCM10 minichromosome maintenance deficient 10 (S. cerevisiae) |
| TBRG4 | 220789_s_at | transforming growth factor beta regulator 4 |
| Pfs2 | 221521_s_at | DNA replication complex GINS protein PSF2 |
| LEF1 | 221558_s_at | lymphoid enhancer-binding factor 1 |
| ZNF45 | 222028_at | zinc finger protein 45 |
| MCM4 | 222036_s_at | MCM4 minichromosome maintenance deficient 4 (S. cerevisiae) |
| MCM4 | 222037_at | MCM4 minichromosome maintenance deficient 4 (S. cerevisiae) |
| CASP8AP2 | 222201_s_at | CASP8 associated protein 2 |
| MGC4692 | 222622_at | Hypothetical protein MGC4692 |
| RAMP | 222680_s_at | RA-regulated nuclear matrix-associated protein |
| FIGNL1 | 222843_at | fidgetin-like 1 |
| SLC25A19 | 223222_at | solute carrier family 25 (mitochondrial deoxynucleotide carrier), member 19 |
| UBE2T | 223229_at | ubiquitin-conjugating enzyme E2T (putative) |
| TCF19 | 223274_at | transcription factor 19 (SC1) |
| PDXP | 223290_at | pyridoxal (pyridoxine, vitamin B6) phosphatase |
| POLR1B | 223403_s_at | polymerase (RNA) I polypeptide B, 128 kDa |
| ANKRD32 | 223542_at | ankyrin repeat domain 32 |
| IL17RB | 224361_s_at | interleukin 17 receptor B /// interleukin 17 receptor B |
| CDCA7 | 224428_s_at | cell division cycle associated 7 /// cell division cycle associated 7 |
| MGC13096 | 224467_s_at | hypothetical protein MGC13096 /// hypothetical protein MGC13096 |
| CDCA5 | 224753_at | cell division cycle associated 5 |
| TMEM18 | 225489_at | transmembrane protein 18 |
| MGC20419 | 225642_at | hypothetical protein BC012173 |
| UHRF1 | 225655_at | ubiquitin-like, containing PHD and RING finger domains, 1 |
| — | 225716_at | Full-length cDNA clone CS0DK008YI09 of HeLa cells Cot 25-normalized of Homo sapiens (human) |
| MGC23280 | 226121_at | hypothetical protein MGC23280 |
| C13orf8 | 226194_at | chromosome 13 open reading frame 8 |
| — | 226832_at | Hypothetical LOC389188 |
| EGR1 | 227404_s_at | Early growth response 1 |
| ZMYND19 | 227477_at | zinc finger, MYND domain containing 19 |
| BARD1 | 227545_at | BRCA1 associated RING domain 1 |
| KIAA1393 | 227653_at | KIAA1393 |
| GPR27 | 227769_at | G protein-coupled receptor 27 |
| RP13-15M17.2 | 228671_at | Novel protein |
| IL17D | 228977_at | Interleukin 17D |
| JPH1 | 229139_at | junctophilin 1 |
| ZNF367 | 229551_x_at | zinc finger protein 367 |
| MGC35521 | 235431_s_at | pellino 3 alpha |
| — | 239312_at | Transcribed locus |
| CSPG5 | 39966_at | chondroitin sulfate proteoglycan 5 (neuroglycan C) |

1. A method of identifying an effective cancer therapy agent for an individual with a platinum-resistant tumor, comprising:
a) Obtaining a cellular sample from the individual;
b) Analyzing said sample to obtain a first gene expression profile;
c) Comparing said first gene expression profile to a platinum chemotherapy responsivity predictor set of gene expression profiles to identify whether said individual will be responsive to a platinum-based therapy;
d) If said individual is an incomplete responder to platinum based therapy, then comparing the first gene expression profile to a set of gene expression profiles comprising at least 5 genes from Table 1 that is capable of predicting responsiveness to other cancer therapy agents;
thereby identifying whether said individual would benefit from the administration of one or more cancer therapy agents,
wherein said cancer therapy agents are not platinum-based.

2. The method of claim 1 wherein the cellular sample is taken from a tumor sample.

3. The method of claim 1 wherein the cellular sample is taken from ascites.

4. The method of claim 1 wherein the cancer therapy agent is a salvage therapy agent.

5. The method of claim 4 wherein the salvage therapy agent is selected from the group consisting of topotecan, adriamycin, doxorubicin, cytoxan, cyclophosphamide, gemcitabine, etoposide, ifosfamide, paclitaxel, docetaxel, and taxol.

6. The method of claim 1 wherein the cancer therapy agent targets a signal transduction pathway that is deregulated.

7. The method of claim 6 wherein the cancer therapy agent is selected from the group consisting of inhibitors of the Src pathway, inhibitors of the E2F3 pathway, inhibitors of the Myc pathway, and inhibitors of the beta-catenin pathway.

8. The method of claim 1 further comprising:
e) Administering to said individual an effective amount of one or more of the cancer therapy agents that was identified in step (d);
thereby treating the individual with said cancer.

9. The method of claim 8 wherein the cancer therapy agent is a salvage agent.

10. The method of claim 9 wherein the salvage therapy agent is selected from the group consisting of topotecan, adriamycin, doxorubicin, cytoxan, cyclophosphamide, gemcitabine, paclitaxel, docetaxel, and taxol.

11. A gene chip for predicting an individual's responsivity to a salvage therapy agent comprising the gene expression profile of at least 5 genes selected from Table 1.

12. A kit comprising a gene chip for predicting an individual's responsivity to a salvage therapy agent comprising the gene expression profile of at least 5 genes selected from Table 1 and a set of instructions for determining an individual's responsivity to salvage therapy agents.

13. A computer readable medium comprising gene expression profiles comprising at least 5 genes from any of Table 1.

14. A method for estimating the efficacy of a therapeutic agent in treating a subject afflicted with cancer, the method comprising:
a) Determining the expression level of multiple genes in a tumor biopsy sample from the subject;
b) Defining the value of one or more metagenes from the expression levels of step (a), wherein each metagene is defined by extracting a single dominant value using singular value decomposition (SVD) from a cluster of genes associated tumor sensitivity to the therapeutic agent; and
c) Averaging the predictions of one or more statistical tree models applied to the values of the metagenes, wherein each model includes one or more nodes, each node representing a metagene, each node including a statistical predictive probability of tumor sensitivity to the therapeutic agent, wherein at least one of the metagenes comprises at least 3 genes in metagenes 1, 2, 3, 4, 5, 6, or 7,
thereby estimating the efficacy of a therapeutic agent in a subject afflicted with cancer.

15. The method of claim 14, wherein step (c) comprises the use of binary regression models.

16. The method of claim 14, further comprising:
d) Administering to the subject an effective amount of a therapeutic agent estimated to be efficacious in step (c),
thereby treating the subject afflicted with cancer.

17. The method of claim 14, wherein said tumor is selected from a breast tumor, an ovarian tumor, and a lung tumor.

18. The method of claim 14, wherein said therapeutic agent is selected from docetaxel, paclitaxel, topotecan, adriamycin, etoposide, fluorouracil (5-FU), and cyclophosphamide, or any combination thereof.

19. The method of claim 14, wherein the therapeutic agent is docetaxel and wherein the cluster of genes comprises at least 10 genes from a metagene selected from any one of metagenes 1 through 7.

20. The method of claim 14, wherein the cluster of genes comprises at least 3 genes.

21. The method of claim 14, wherein at least one of the metagenes is metagene 1, 2, 3, 4, 5, 6, or 7.

22. The method of claim 14, wherein the cluster of genes corresponding to at least one of the metagenes comprises 3 or more genes in common to metagene 1, 2, 3, 4, 5, 6, or 7.

23. The method of claim 14, wherein step (a) comprises extracting a nucleic acid sample from the sample from the subject.

24. The method of claim 14, wherein the expression level of multiple genes in the tumor biopsy sample is determined by quantitating nucleic acids levels of the multiple genes using a DNA microarray.

25. The method of claim 14, wherein at least one of the metagenes shares at least 50% of its defining genes in common with metagene 1, 2, 3, 4, 5, 6, or 7.
